(19) United States
(12) Patent Application Publication
(10) Pub. No.: US 2026/0337263 A1
(43) Pub. Date: Oct. 29, 2026

(54) ANTIBODIES AGAINST MSLN AND METHODS OF USE THEREOF

(71) Applicant: Dana-Farber Cancer Institute, Inc., Boston, MA (US)

(72) Inventors: Wayne A. Marasco, Wellesley, MA (US); Matthew Chang, Brookline, MA (US)

(21) Appl. No.: 19/104,336

(22) PCT Filed: Aug. 15, 2023
(86) PCT No.: PCT/US2023/030268

§ 371 Date: Feb. 17, 2025

Related U.S. Application Data
(60) Provisional application No. 63/398,082, filed on Aug. 15, 2022.

(51) Int. Cl. C07K 16/28; A61K 40/11; A61K 40/31; A61K 40/33; A61K 40/42; A61P 35/00

(52) U.S. Cl. CPC C07K 16/28; A61K 40/11; A61K 40/31; A61K 40/33; A61K 40/4255; A61P 35/00; A61K 2239/13; A61K 2239/29; C07K 2317/31; C07K 2317/52; C07K 2317/565; C07K 2317/622; C07K 2317/76

(57) ABSTRACT

The present invention comprises human antibodies that bind to mesothelin (MSLN) protein. The antibodies can be used to treat cancer.

This application is an International Application which claims priority from U.S. provisional patent application No. 63/398,082, filed on Aug. 15, 2022, the entire contents of which are incorporated herein by reference.

All patents, patent applications and publications cited herein are hereby incorporated by reference in their entirety. The disclosures of these publications in their entireties are hereby incorporated by reference into this application to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### FIELD OF THE INVENTION

This invention comprises antibodies against Mesothelin (MSLN) and methods of use thereof.

### BACKGROUND

Mesothelin (MSLN) is a protein normally expressed in mesothelial cells. MSLN can exist in two forms, bound to the cell membrane via a GPI linker, and in a soluble/shed form.

### SUMMARY OF THE INVENTION

Aspects of the invention are drawn towards an isolated monoclonal antibody or antigen-binding fragment thereof that binds to a mesothelin protein or fragment thereof comprising a heavy chain, light chain, or a combination thereof. In embodiments, the heavy chain comprises a CDR1 comprising GYTLTTNG (SEQ ID NO: 31), GGTFSSDT (SEQ ID NO: 35), GFTFDDYA (SEQ ID NO: 38), GFKFTDYY (SEQ ID NO: 46), or GYTFTHYA (SEQ ID NO: 49), CDR2 comprising VSPYNGHI (SEQ ID NO: 32), VNPYNGHI (SEQ ID NO: 34), INPNSGGT (SEQ ID NO: 36), ISWNSGSI (SEQ ID NO: 39), ISWNNGSI (SEQ ID NO: 41), ISWNSGNI (SEQ ID NO: 43), INTSSNHI (SEQ ID NO: 47), or IHAGNGNS (SEQ ID NO: 50), CDR3 comprising ARVNRANYYGMDV, (SEQ ID NO: 33), ARESALGGSYPLSF (SEQ ID NO: 37), AKDPSSSWLAGAFDI (SEQ ID NO: 40), AKDPSTSWLAGAFDI (SEQ ID NO: 42), AKDAGSSGYFNAFDI (SEQ ID NO: 44), AKSPSSNWYPDAFDI (SEQ ID NO: 45), ARGASWGPL (SEQ ID NO: 48), or AREVGHGMDV (SEQ ID NO: 51), or a combination of CDRs thereof; wherein the light chain comprises a CDR1 comprising QSLLRSNGYNY (SEQ ID NO: 52), QSVSSSY (SEQ ID NO: 59), SLRRFY (SEQ ID NO: 62), SLIRFY (SEQ ID NO: 65), SLRAYY (SEQ ID NO: 67), SLRNYY (SEQ ID NO: 70), NIGSKS (SEQ ID NO: 73), or SSNIGAGYD (SEQ ID NO: 76), CDR2 comprising LGS (SEQ ID NO: 53), GAS (SEQ ID NO: 60), GKN (SEQ ID NO: 63), AKN (SEQ ID NO: 71), DDS (SEQ ID NO: 74), or GNT (SEQ ID NO: 77), CDR3 comprising MQSSTNSAH (SEQ ID NO: 54), MQALQTPLT (SEQ ID NO: 55), MQALQTPL (SEQ ID NO: 56), MQALQTPLT (SEQ ID NO: 57), HASSTNSAH (SEQ ID NO: 58), LQDDSYPLT (SEQ ID NO: 61), NSRDSDGNHVF (SEQ ID NO: 64), NSQDRDGNHVF (SEQ ID NO: 66), NSRDSSGNHLGV (SEQ ID NO: 68), NSRDSSGNHLGS (SEQ ID NO: 69), SSRDSSDNHVV (SEQ ID NO: 72), QSADYNWLCD (SEQ ID NO: 7475), or QSYDSSLSGYV (SEQ ID NO: 78), or a combination of CDRs thereof. In embodiments, the sequences have been determined using IMGT numbering scheme.

In embodiments, the mesothelin protein is a human mesothelin protein.

In embodiments, the antibody is fully human or humanized.

In embodiments, the antibody is monospecific, bispecific, or multispecific.

In embodiments, the antibody is an IgG. For example, the antibody is an IgG1, IgG2, IgG3, or IgG4 antibody.

In embodiments, the antibody is a single chain antibody, bispecific antibody, or bispecific T cell engager.

In embodiments, the antibody has a binding affinity of at least 1.0×10-9 M.

In embodiments, the antibody further comprising a heavy chain constant region, a light chain constant region, an Fc region, or a combination thereof.

In embodiments, the antibody comprises Gly3-1-H10, TEA2-E10, TEA1-D3, Gly3-2-C1, TEA1-D2, Gly2-2-F7, Gly 1-2-H4, Gly1-2-D4, TEA1-E5, Gly3-2-C10, Gly2-1-F2, Gly2-2-B6, Gly1-2-E10, TEA2-C9, or TEA1-E8.

In embodiments, the antibody competes with binding of Gly3-1-H10, TEA2-E10, TEA1-D3, Gly3-2-C1, TEA1-D2, Gly2-2-F7, Gly1-2-H4, Gly 1-2-D4, TEA1-E5, Gly3-2-C10, Gly2-1-F2, Gly2-2-B6, Gly 1-2-E10, TEA2-C9, or TEA1-E8.

In embodiments, the antibody competes with binding of Gly3-1-H10, TEA2-E10, TEA1-D3, Gly3-2-C1, TEA1-D2, Gly2-2-F7, Gly 1-2-H4, Gly 1-2-D4, TEA1-E5, Gly3-2-C10, Gly2-1-F2, Gly2-2-B6, Gly1-2-E10, TEA2-C9, or TEA1-E8.

In embodiments, the antibody or fragment is linked to a therapeutic agent.

In embodiments, the antibody is a single chain fragment, a bispecific antibody, or a bispecific T cell engager (BiTE).

Aspects of the invention are also drawn towards an isolated antibody or fragment thereof that binds to a human mesothelin protein. In embodiments, the antibody comprises: (a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or (b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or (c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or (d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or (e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or (f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or (g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or (h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or (i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; (j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66; (k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68; (1) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69; (m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72; (n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or (o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

Aspects of the invention are also drawn towards an isolated scFv antibody that binds to a human mesothelin protein. In embodiments, the antibody comprises: (a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or (b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or (c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or (d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or (e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or (f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or (g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or (h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or (i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; (j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66; (k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68; (1) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69; (m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72; (n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or (o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78. In embodiments, the sequences have been determined using IMGT numbering scheme.

In embodiments, the invention comprises an isolated antibody or fragment thereof that binds to a human mesothelin protein comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto.

In embodiments, the invention comprises an isolated antibody or fragment thereof that binds to a human mesothelin protein comprising a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

In embodiments, the invention comprises an isolated antibody or fragment thereof that binds to a human mesothelin protein comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

In embodiments, the invention comprises an isolated scFv that binds to a human mesothelin protein comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto.

In embodiments, the invention comprises an isolated scFv that binds to a human mesothelin protein comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

In embodiments, the invention comprises an isolated scFv that binds to a human mesothelin protein comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

In embodiments, the invention comprises an isolated monoclonal antibody or antigen-binding fragment thereof that binds to a human mesothelin protein comprising a heavy chain, a light chain, or a combination thereof, wherein the heavy chain comprises an amino acid sequence about 95% identical to SEQ ID NO: 1, and the light chain comprises an amino acid sequence about 95% identical to SEQ ID NO: 2.

In embodiments, the invention comprises an isolated scFv that binds to a human mesothelin protein comprising a heavy chain, a light chain, or a combination thereof, wherein the heavy chain comprises an amino acid sequence about 95% identical to SEQ ID NO: 1, and the light chain comprises an amino acid sequence about 95% identical to SEQ ID NO: 2.

In embodiments, the invention comprises an isolated monoclonal antibody or antigen-binding fragment thereof that binds to a human mesothelin protein comprising a heavy chain, a light chain, or a combination thereof, wherein the heavy chain comprises an amino acid sequence about 95% identical to SEQ ID NO: 3, and the light chain comprises an amino acid sequence about 95% identical to SEQ ID NO: 4.

In embodiments, the invention comprises an isolated scFv antibody that binds to a human mesothelin protein comprising a heavy chain, a light chain, or a combination thereof, wherein the heavy chain comprises an amino acid sequence about 95% identical to SEQ ID NO: 3, and the light chain comprises an amino acid sequence about 95% identical to SEQ ID NO: 4.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 5, and the light chain comprises an amino acid sequence about 95% identical to SEQ ID NO: 6.

In embodiments, the invention comprises an isolated scFv that binds to a human mesothelin protein, comprising a heavy chain, a light chain, or a combination thereof, wherein the heavy chain comprises an amino acid sequence about 95% identical to SEQ ID NO: 5, and the light chain comprises an amino acid sequence about 95% identical to SEQ ID NO: 6.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 7, and the light chain comprises an amino acid sequence about 95% identical to SEQ ID NO: 8.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 7, and the light chain comprises an amino acid sequence about 95% identical to SEQ ID NO: 8.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 9, and the light chain comprises an amino acid sequence about 95% identical to SEQ ID NO: 10.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 9, and the light chain comprises an amino acid sequence about 95% identical to SEQ ID NO: 10.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 11, and the light chain comprises an amino acid sequence about 95% identical to SEQ ID NO: 12.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 11, and the light chain comprises an amino acid sequence about 95% identical to SEQ ID NO: 12.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 13, and the light chain comprises an amino acid sequence about 95% identical to SEQ ID NO: 14.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 13, and the light chain comprises an amino acid sequence about 95% identical to SEQ ID NO: 14.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 15, and the light chain comprises an amino acid sequence about 95% identical to SEQ ID NO: 16.

In embodiments, the invention comprises an isolated scFv that binds to a human mesothelin protein comprising a heavy chain, a light chain, or a combination thereof, wherein the heavy chain comprises an amino acid sequence about 95% identical to SEQ ID NO: 15, and the light chain comprises an amino acid sequence about 95% identical to SEQ ID NO: 16.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 17, and the light chain comprises an amino acid sequence about 95% identical to SEQ ID NO: 18.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 17, and the light chain comprises an amino acid sequence about 95% identical to SEQ ID NO: 18.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 19, and the light chain comprises an amino acid sequence about 95% identical to SEQ ID NO: 20.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 19, and the light chain comprises an amino acid sequence about 95% identical to SEQ ID NO: 20.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 21, and the light chain comprises an amino acid sequence about 95% identical to SEQ ID NO: 22.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 21, and the light chain comprises an amino acid sequence about 95% identical to SEQ ID NO: 22.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 23, and the light chain comprises an amino acid sequence about 95% identical to SEQ ID NO: 24.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 23, and the light chain comprises an amino acid sequence about 95% identical to SEQ ID NO: 24.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 25, and the light chain comprises an amino acid sequence about 95% identical to SEQ ID NO: 26.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 25, and the light chain comprises an amino acid sequence about 95% identical to SEQ ID NO: 26.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 27, and the light chain comprises an amino acid sequence about 95% identical to SEQ ID NO: 28.

In embodiments, the invention comprises an isolated scFv that binds to a human mesothelin protein, comprising a heavy chain, a light chain, or a combination thereof, wherein the heavy chain comprises an amino acid sequence about 95% identical to SEQ ID NO: 27, and the light chain comprises an amino acid sequence about 95% identical to SEQ ID NO: 28.

In embodiments, the invention comprises an isolated monoclonal antibody or antigen-binding fragment thereof that binds to a human mesothelin protein, comprising a heavy chain, a light chain, or a combination thereof, wherein the heavy chain comprises an amino acid sequence about 95% identical to SEQ ID NO: 29, and the light chain comprises an amino acid sequence about 95% identical to SEQ ID NO: 30.

In embodiments, the invention comprises an isolated scFv antibody that binds to a human mesothelin protein, comprising a heavy chain, a light chain, or a combination thereof, wherein the heavy chain comprises an amino acid sequence about 95% identical to SEQ ID NO: 29, and the light chain comprises an amino acid sequence about 95% identical to SEQ ID NO: 30.

Aspects of the invention are also drawn towards an isolated bispecific antibody, comprising the antibody fragment as described herein and a second antigen-binding fragment having specificity to a molecule on an immune cell. For example, the molecule is selected from the group consisting of CCR4, B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, and KIR.

In embodiments, the fragment and the second fragment each is independently selected from a Fab fragment, a single-chain variable fragment (scFv), or a single-domain antibody.

In embodiments, the bispecific antibody further comprises a Fc fragment.

Still further, aspects of the invention are drawn towards a bispecific T cell engager (BiTE) that binds to a human mesothelin protein. In embodiments, the BiTE comprises: (a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or (b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or (c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or (d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or (e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or (f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or (g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or (h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or (i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; (j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66; (k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68; (1) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69; (m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72; (n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or (0) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78. In embodiments, the sequences have been determined using IMGT numbering scheme.

Also, aspects of the invention are drawn towards a bispecific T cell engager (BiTE) that binds to a human mesothelin protein, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto.

Further, aspects of the invention are drawn towards a bispecific T cell engager (BiTE) that binds to a human mesothelin protein, comprising a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

Still further, aspects of the invention are drawn towards a bispecific T cell engager (BiTE) that binds to a human mesothelin protein, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

Aspects of the invention are also drawn towards a nucleic acid encoding the antibody or fragment as described herein.

Aspects of the invention are also drawn towards a pharmaceutical composition comprising the antibody or fragment thereof as described herein, and a pharmaceutically acceptable carrier or excipient.

In embodiments, the pharmaceutical composition further comprises at least one additional therapeutic agent. For example, the therapeutic agent is a toxin, a radiolabel, a siRNA, a small molecule, or a cytokine.

Aspects of the invention are also drawn towards an isolated cell comprising one or more polynucleotide(s) encoding an antibody or fragment as described herein.

In embodiments, a vector can comprise the nucleic acid as described herein.

In embodiments, a cell can comprise the vector as described herein.

Aspects of the invention are also drawn towards an engineered cell comprising a chimeric antigen receptor. In embodiments, the chimeric antigen receptor comprises an extracellular ligand binding domain that is specific for an antigen on the surface of a cancer cell, wherein the antigen comprises mesothelin, wherein the extracellular ligand binding domain comprises an antibody or fragment thereof, where the antibody or fragment thereof comprises: (a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; (b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; (c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; (d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; (e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; (f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; (g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; (h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; (i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; (j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66; (k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68; (1) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69; (m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72; (n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or (o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78. In embodiments, the sequences have been determined using IMGT numbering scheme.

In embodiments, the antibody or fragment thereof comprises a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto.

In embodiments, the antibody or fragment thereof comprises a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

In embodiments, the antibody or fragment thereof comprises a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

In embodiments, the cell comprises a T cell, an NK cell, an NKT cell, an iPS cell, an iPS-derived cell, a cell line, or a B cell. For example, the cell comprises a CD4+, CD8+, CD3+ pan T cells, or any combination thereof.

Aspects of the invention are also drawn towards a kit. In embodiments, the kit comprises at least one pharmaceutical composition as described herein; a syringe, needle, or applicator for administration of the at least one antibody to a subject; and instructions for use.

Further, aspects of the invention are drawn towards a method for detecting the presence of mesothelin in a sample. In embodiments, the method comprises contacting the sample with the monoclonal antibody as described herein and detecting the presence or absence of an antibody-antigen complex, thereby detecting the presence of mesothelin in the sample.

In embodiments, contacting comprises immunohistochemistry. For example, immunohistochemistry comprises precipitation, immunofluorescence, western blot, or ELISA.

In embodiments, the sample comprises whole blood, a blood component, a body fluid, a biopsy, a tissue, serum or one or more cells. For example, the one or more cells comprise an in vitro culture. For example, the one or more cells comprise mesothelin-expressing cells.

In embodiments, the sample comprises a normal sample or a cancerous sample. For example, the sample is an in vitro sample.

Embodiments can further comprise the step of obtaining a sample from a subject.

In embodiments, the cancer expresses mesothelin. In embodiments, the cancer comprises an epithelial cell cancer. For example, the epithelial cell cancer comprises lung cancer, mesothelioma, or ovarian cancer.

Aspects of the invention are also drawn towards a method for treating cancer in a subject. In embodiments, the method comprises administering to the subject a pharmaceutical composition as described herein.

In embodiments, the cancer comprises an epithelial cell cancer. For example, the cancer comprises lung cancer, mesothelioma or ovarian cancer.

Other objects and advantages of this invention are readily apparent from the ensuing description.

### BRIEF DESCRIPTION OF THE FIGURES

FIG. 1 is a characterization of a set of antibodies for CASS B cell development. FIG. 1 provides cell binding curve for anti-MSLN (top) and anti-Muc1 (bottom) scFv-Fcs indicate that we have previously identified high affinity antibodies.

FIG. 2 is a CASS B cell construct testing and B cell transduction. Panel A shows cells expressing the engineered IgG-BCR construct can bind soluble HA. Panel B shows lentivirus can be used to achieve high transduction efficiency using multiple DNA constructs and donors. Panel C shows an engineered reporter system in Jurkat cells using an NFAT/NFkB inducible response element that shows increasing levels of GFP expression with various stimulatory conditions. Embodiments as described herein can be used in the CASS B cell platform.

FIG. 3 shows 293T cells transfected with a engineered membrane bound IgG show high levels of membrane IgG binding (Panel A) and soluble HA capture (Panel B).

FIG. 4 is a schematic showing the structure of mesothelin (MSLN). Adapted from Mesothelin, Hassan, Raffit et al. Clin Cancer Res, 2004 and New High Affinity Monoclonal Antibodies Recognize Non-Overlapping Epitopes On Mesothelin For Monitoring And Treating Mesothelioma, Zhang, Yi-Fan et al. Scientific Reports, 2015.

FIG. 5 is a schematic showing a protocol for whole cell panning.

FIG. 6 is a table showing Mesothelin panning results.

FIG. 7 shows representative FACS data and FACS screening summary (14 plates). Picked 14×96 well plates of colonies; 3× Gly wash 1; 3× Gly wash 2; 4× Gly wash 3; 4× TEA elution; FACS with 100K 293T-MSLN+ cells (blue) or 100K 293-MSLN-cells (red)

FIG. 8 shows MSLN serum levels. Adapted from Detection and Quantitation of Serum Mesothelin, a Tumor Marker for Patients with Mesothelioma and Ovarian Cancer, Hassan, Raffit, et al, Clin Cancer Res, 2006 and A prospective trial evaluating the role of mesothelin in undiagnosed pleural effusions, Hooper, Clare E. et al, European Respiratory Journal, 2013

FIG. 9 shows purified phage binding curves (MSLN).

FIG. 10 shows kinetic data (raw supernatant). scFv-Fcs were expressed in a 6 well plate using Expi293 cells. Two days after transfection, scFv-Fc concentration in the supernatant was quantified via Bethyl IgG quantification kit. These concentrations were used to normalize samples to 10 ug/ml for Octet kinetic analysis. Anti-human-Fc sensors were loaded with the scFv-Fc from diluted supernatant and soluble recombinant mesothelin (Biolegend) was allowed to bind, followed by a disassociation step in PBST. Kinetic constants are shown above. Samples were run using raw supernatant and in singlet.

FIG. 11 shows purified scFv-Fc binding curves.

FIG. 12 shows killing assay with 293T-MSLN-BFP cells and 293T-CLDN4-mCard cells mixed.

FIG. 13 shows killing assay with 293T-MSLN-BFP cells and 293T-CLDN4-mCard cells mixed.

FIG. 14 shows killing assay of anti-MSLN mono CAR T cells against Ovcar8 and Ovcar5 cells.

FIG. 15 and FIG. 16 show anti-MSLN CAR T cells were incubated at a 5:1 ratio with a 1:1 mixture of ovarian cancer cell lines expressing high (Ov8) or low (Ov5) levels of MSLN for 48 hours. Number of Ov8 and Ov5 cells were counted at T0 and T48 using the Celigo image cytometer. Cytotoxic CAR T cells eliminate Ov8 cells (solid lines) more efficiently than the low MSLN+ Ov5 (dashed lines) cells. These two figures are the same experiment, just different plates testing different antibody/CAR T constructs.

FIG. 17 shows killing assay with sorted cells. Anti-MSLN CART cells were sorted and expanded prior to killing assay. Positive numbers indicate killing, negative numbers indicate target cell growth/expansion. As demonstrated here, the anti-MSLN CAR T cells exhibit preferential killing of MSLN+ OVCAR8 cells and not MSLN-Skrc59 cells.

FIG. 18 shows CRISPR knockout MSLN in Ovcar8.

FIG. 19 shows protocol for dual CAR T E8-T4E3 killing assay.

FIG. 20 shows dual CAR T E8-T4E3 killing assay. CARTS of donors 2020030401 were sorted and CARTS of donors 2020030402 were not sorted.

FIG. 21 shows F10 nonspecific killing is cell line dependent.

FIG. 22 shows F10 nonspecific killing is donor dependent.

FIG. 23 shows killing assay of Ovcar8 MSLN negative cell line.

FIG. 24 shows killing assay of Ovcar8 MSLN negative cell line.

FIG. 25 shows COV362 binding curve.

FIG. 26 shows Ovcar8 binding curve.

FIG. 27 shows MSLN surface expression on Ovcar8 is higher than that of Cov362.

FIG. 28 shows the anti-MSLN CAR T cells exhibit preferential killing of MSLN+ OVCAR8 cells and not MSLN-Skrc59 cells. Anti-MSLN CART cells were sorted and expanded prior to killing assay. Positive numbers indicate killing, negative numbers indicate target cell growth/expansion.

FIG. 29 shows antibody competition. anti-His HISIK sensors were used for competition. Sensors were loaded with 5 ug/ml MSLN-his (Biolegend) for 60 seconds. YP158 or YP218 were then loaded onto the sensors until receptor saturation. Following a brief baseline reading, the sensors were dipped into wells containing the 8 aMSLN antibodies.

FIG. 30 shows killing of additional MSLN+ OvCA cell lines with anti-MSLN E8 CAR T.

FIG. 31 shows killing assay of anti-muc1 and anti-msln dual CAR T. CAR T tested: E8-T4E3: anti-mesothelin and anti-muc1 dual CAR T; E8: anti-mesothelin mono-CAR T; T4E3: anti-MUC1 mono-CAR T; F10: anti-HA mono-CAR T as negative control. In vitro killing protocol using Celigo: 1500 BFP labeled 293msln+ cells were mixed with 1500 mCardinal labeled Cov362msln+/MUC1+ cells. Individual CART was added at E:T=2:1. Data was collected 42 hours after adding of CAR T to tumor cells. Percent Killing based on: Panel A; 293msln+; Panel B: Cov362 (msln+/MUC1+).

FIG. 32 shows a schematic of anti-mesothelin CASS B cell therapy for NSCLC.

FIG. 33 shows characterization of non-limiting, exemplary antibodies for CASS B cell development. A cell binding curve (left) and BLI based kinetic measurements (right) demonstrate that one clone (Gly1-2-H4) binds a conformational epitope only present on the GPI linked form.

FIG. 34 shows schematic of engineering strategy for dual targeting and affinity fine-tuning of anti-MUC1/anti-MSLN scFv targeting moieties to increase tumor killing efficacy and patient safety. Balanced affinity, and scFv orientation to both targets will provide the optimized DFIR CAR targeting design.

FIG. 35 shows anti-MSLN CAR killing. Panel A provides a schematic of 71 kDa surface glycosylphosphatidylinositol (GPI)-linked glycoprotein that is processed to 31 kDa megakaryocyte potentiating factor (MPF) and the 40 kDa MSLN that remains membrane bound; Panels B & C), Killing of MSLN+ OVCAR8 cells (Panel B) but not SK59 RCC cells (Panel C) by three (H4, F2 & E8) anti-MSLN CAR-T cells; Panel D), Killing of MUC1+, MSLN+ CoV362 cells by each anti-MUC1 (T4E3) and anti-MSLN (E8) CAR-T cells and the dual targeted E8-TAE3 CAR-T cells.

FIG. 36 shows anti-tumor effects of anti-CCR4 mAb 2-3 in vivo. Panel A shows mAb2-3 treatment blocks migration of human Tregs to CCL22 secreting OvCA cells. Panel B shows mAb2-3 treatment in vivo restores killing of IGROV-1 by depletion of Tregs.

FIG. 37 shows schematic of DFIR CAR. Anti-MUC1 and anti-MLSN targeting moieties will be tested in different positions and with different lengths to optimize orientation-based access to their respective targets. An internal ribosomal entry site (IRES) will allow CAP independent translation of Ab payloads.

FIG. 38 shows evaluating CAR-T cell killing in ccRCC 3D cultures. Panel A provides IF staining of Hoechst (in blue), Calcein and EpCAM (in green), PI and CD8 (in red), CD45, EpCAM and CAIX (in purple) on ccRCC patient derived organotypic spheroids (PDOTS). Panel B provides a workflow of generating ccRCC PDOTS. Panel C provides T cell migration and cytokine release. Anti-CAIX G36 CAR-T cells (express ZsGreen) were evaluated in ccRCC PDOTS and anti-BCMA A716 (control) CAR-T cells, untransduced T cells and no treatment were used as negative controls. T cell migration from side channels to the center channel was observed and quantified by using ZsGreen. CXCL10 release was assessed via ELISA.

FIG. 39 shows antibody binding data. FACS binding represents cell expressed MSLN and ELISA binding represents soluble MSLN. This distinction can be important because MSLN can be shed from tumor cells, acting as a decoy for CAR T/Ab therapies; however, there is a slight conformational shift upon shedding that can allow for targeting of membrane bound MSLN only.

FIG. 40 shows amino acid sequences of anti-mesothelin antibodies.

### DETAILED DESCRIPTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Unique recombinant monoclonal mesothelin (MSLN) antibodies are described herein. “Recombinant” as it pertains to polypeptides (such as antibodies) or polynucleotides can refer to a form of the polypeptide or polynucleotide that does not exist naturally, a non-limiting example of which can be created by combining polynucleotides or polypeptides that do not normally occur together.

The amino acid sequence of the monoclonal MSLN antibodies are provided herein, in addition to an exemplary wildtype IgG constant region useful in combination with the VH and VL sequences provided herein (see Tables 1-15); the amino acid sequences of the heavy and light chain complementary determining regions CDRs of the MSLN antibodies are underlined (CDR1), underlined and bolded (CDR2), or underlined, italicized, and bolded (CDR3) below:

TABLE 1
MSLN-WC-R3-Gly3-1-H10 Ab Variable Region amino acid sequences
V_{H} chain of Gly3-1-H10
QVQLVQSGAEVKKPGASVRVSCKASGYTLTTNGLSWVRQAPGHGLEWMGWVSPYNGHI
SYARNLEGRITMTTDTSTNTAHMELRRLTSDDTAVYYCARVNRANYYGMDVWGQGTTVT
VSS
(SEQ ID NO: 1)
V_{L} chain of Gly3-1-H10
EIVLTQSPLSLPVTPGEPASISCRSSQSLLRSNGYNYLDWYLQKPGQSPQLLIYLGSNRASGV
PDRFSASGSGTDFTLKISRVEAEDVGVYYCMQSSTNSAHFGGGTKVEIK
(SEQ ID NO: 2)

TABLE 2
MSLN-WC-R3-TEA2-E10 Ab Variable Region amino acid sequences
V_{H} chain of TEA2-E10
QVQLVQSGAEVKKPGASVRVSCKASGYTLTTNGLSWVRQAPGHGLEWMGWVSPYNGHI
SYARNLEGRITMTTDTSTNTAHMELRRLTSDDTAVYYCARVNRANYYGMDVWGQGTTVT
VSS (SEQ ID NO: 3)
V_{L} chain of TEA2-E10
EIVLTQSPLSLPVTPGEPASISCRSSQSLLRSNGYNYLDWYLQKPGQSPQLLIYLGSNRASGV
PDRFSASGSGTDFTLKISRVEAEDVGVYYCMQSSTNSAHFGGGTKVEIK
(SEQ ID NO: 4)

TABLE 3
MSLN-WC-R3-TEA1-D3 Ab Variable Region amino acid sequences
V_{H} chain of TEA1-D3
QVQLVQSGP.EVKKPGASLRVSCKASGYTLTTNGLTWVRQAPGHGLEWMGWVNPYNGHI
NYARNLKGRITMTTDTSTNTAHMELRRLTSDDTAVYYCARVNRANYYGMDVWGQGTTVT
VSS
(SEQ ID NO: 5)
V_{L} chain of TEA1-D3
EIVLTHSPLSLPVTPGKPASISCRSSQSLLRSNGYNYLDWYLHNPGQSPQLLIYLGSIRASGVP
DRFSASGSRTDFTLKISKEDADDVGVYYCMQALQTPLTFSGGTNVEIK
(SEQ ID NO: 6)

TABLE 4
MSLN-WC-R3-Gly3-2-C1 Ab Variable Region amino acid sequences
V_{H} chain of Gly3-2-C1
QVQLVQSGAEVKKPGASVRVSCKASGYTLTTNGLSWVRQAPGHGLEWMGWVSPYNGHI
SY ARNLEGRITMTTDTSTNTAHMELRRLTSDDTAVYYCARVNRANYYGMDVWGQGTTVT
VSS
(SEQ ID NO: 7)
V_{L} chain of Gly3-2-C1
EIVLTQSPLSLPVTPGEPASISCRSSQSLLRSNGYNYLDWYLQKPGQSPQLLIYLGSNRASGV
PDRFSASGSCTDFTLKISRVEAEDVGVYYCMQALQTPLFRGGTKVEIK
(SEQ ID NO: 8)

TABLE 5
MSLN-WC-R3-TEA1-D2 Ab Variable Region amino acid sequences
V_{H} chain of TEA1-D2
QVQLVQSGAEVKKPGASVRVSCKASGYTLTTNGLSWVRQAPGHGLEWMGWVSPYNGHI
SYARNLEGRITMTTDTSTNTAHMELRRLTSDDTAVYYCARVNRANYYGMDVWGQGTTVT
VSS
(SEQ ID NO: 9)
V_{L} chain of TEA1-D2
EIVLTQSPLSLPVTPGEPASISCRSSQSLLRSNGYNYLDWYLQKPGQSPQLLIYLGSNRASGV
PDRFSASGSGTDFTLKISRVEAEDVGVYYCMQALQTPLTFGGGTKVEIK
(SEQ ID NO: 10)

TABLE 6
MSLN-WC-R3-Gly2-2-F7 Ab Variable Region amino acid sequences
V_{H} chain of Gly 2-2-F7
QVQLVQSGAEVKKPGASVRVSCKASGYTLTTNGLSWVRQAPGHGLEWMGWVSPYNGHI
SYARNLEGRITMTTDTSTNTAHMELRRLTSDDTAVYYCARVNRANYYGMDVWGQGTTVT
VSS
(SEQ ID NO: 11)
V_{L} chain of Gly2-2-F7
EIVLTQSPLSLPVTPGEPASISCRSSQSLLRSNGYNYLDWYLQKPGQSPQLLIYLGSNRASGV
PDRFSASGSGTDFTLKISRVEAEDVGGLLHASSTNSAHFGGGTKVEIK
(SEQ ID NO: 12)

TABLE 7
MSLN-WC-R3-Gly1-2-H4 Ab Variable Region amino acid sequences
V_{H} chain of Gly 1-2-H4
QVQLVQSGAEVKKPGSSVKVSCKSSGGTFSSDTINWVRQAPGQGLEWMGWINPNSGGTN
YAQKFQGWVTMTRDTSISTAYMELSRLRSDDTAVYYCARESALGGSYPLSFWGQGTLVTV
SS
(SEQ ID NO: 13)
V_{L} chain of Gly 1-2-H4
ETTLTQSPGTLSLSPGERATLSCRASQSVSSSYLAWYQQKPGQAPRLLIYGASSRATGIPDRF
SGSGSGTDFTLTISRLEPEDSATYYCLQDDSYPLTFGQGTKVEIK
(SEQ ID NO: 14)

TABLE 8
MSLN-WC-R3-Gly1-2-D4 Ab Variable Region
amino acid sequences

V_{H} chain of Gly 1-2-D4
EVQLVQSGGGLVQPGRSLRLSCAASGFTFDDYAMHWVRQAPGKGLEWV
SGISWNSGSIGYADSVKGRFTISRDNAKNSLYLQMNSLRAEDTALYYC
AKDPSSSWLAGAFDIWGQGTMVTVSS
(SEQ ID NO: 15)
V_{L} chain of Gly 1-2-D4
SSELTQDPTVSVALGQTVRITCQGDSLRRFYASWYQQKPGQAPILVIY
GKNNRPSGIPDRFSGSYSGNTASLTITGAQAEDEADYYCNSRDSDGNH
VFFGGGTKLTVL
(SEQ ID NO: 16)

TABLE 9
MSLN-WC-R3-TEA1-E5 Ab Variable Region
amino acid sequences

V_{H} chain of TEA1-E5
EVQLVQSGGGLVQPGRSLRLSCAASGFTFDDYAMHWVRQAPGKGLEWV
SGISWNSGSIGYADSVKGRFTISRDNAKNSLYLQMNSLRAEDTALYYC
AKDPSSSWLAGAFDIWGQGTMVTVSS
(SEQ ID NO: 17)
V_{L} chain of TEA1-E5
SSELTQDPTVSVALGQTVRITCQGDSLRRFYASWYQQKPGQAPILVIY
GKNNRPSGIPDRFSGSYSGNTASLTITGAQAEDEADYYCNSRDSDGNH
VFFGGGTKLTVL
(SEQ ID NO: 18)

TABLE 10
MSLN-WC-R3-Gly3-2-C10 Ab Variable Region
amino acid sequences

V_{H} chain of Gly3-2-C10
EVQLVQSGGGLIQPGGSLRLSCAASGFTFDDYAMHWVRQAPGKGLEWM
SWISWNNGSIGYADSVK.GRFPISRNNPQNSLYLQMNSLKAEDTALYY
CAKDPSTSWLAGAFDIWGQGTMVTVSS
(SEQ ID NO: 19)
V_{L} chain of Gly 3-2-C10
SSDLTLDPTVSVALGHTVRITCQGDSLIRFYASWYHQEPGPAPILVIY
GKNTRPSRIPDLFSGSYSRNTSSLTISGAQVKDEADYYCNSQDRDGNH
VFFGGGTKLTVL
(SEQ ID NO: 20)

TABLE 11
MSLN-WC-R3-Gly2-1-F2 Ab Variable Region
amino acid sequences

V_{H} chain of Gly2-1-F2
EVQLVQSGGGLVQPGRSLRLSCAASGFTFDDYAMHWVRQAPGKGLEWV
SGISWNSGNIGYADSVK.GRFTISRDNAKNSLYLQMNSLRAEDTALYY
CAKDAGSSGYFNAFDIWGQGTMVTVSS
(SEQ ID NO: 21)
V_{L} chain of Gly2-1-F2
SSELTQDPAVSVALGQTVRITCQGDSLRAYYASWYQQKPGQAPVLVIY
GKNSRPSGIPDRFSGSNSGNAASLTITGAQAEDEADYYCNSRDSSGNH
LGVFGGGTKLTVL
(SEQ ID NO: 22)

TABLE 12
MSLN-WC-R3-Gly2-2-B6 Ab Variable Region
amino acid sequences

V_{H} chain of Gly2-2-B6
EVQLVQSGGGLVQPGRSLRLSCAASGFTFDDYAMHWVRQAPGKGLEWV
SGISWNSGNIGYADSVKGRFTISRDNAKNSLYLQMNSLRAEDTALYYC
AKDAGSSGYFNAFDIWGQGTMVTVSS
(SEQ ID NO: 23)
V_{L} chain of Gly2-2-B6
SSELTQDPAVSVALGQTVRITCQGDSLRAYYASWYQQKPGQAPVLVIY
GKNSRPSGIPDRFSGSNSGNAASLTITGAQAEDEADYYCNSRDSSGNH
LGSFGGGTKLTVL
(SEQ ID NO: 24)

TABLE 13
MSLN-WC-R3-Gly1-2-E10 Ab Variable Region
amino acid sequences

V_{H} chain of Gly 1-2-E10
QVQLVQSGGGLVQPGRSLRLSCAASGFTFDDYAMHWVRQGPGKGLEWV
SGISWNSGSIGFADSVKGRFTISRDNAKNSLYLQMNSLRVEDTALYYC
AKSPSSNWYPDAFDIWGQGTMVTVSS
(SEQ ID NO: 25)
V_{L} chain of Gly 1-2-E10
SSELTQDPAVSVALGQTVRITCQGDSLRNYYASWYQQKPGQAPVLVIY
AKNNRPSGIPDRFSGSSSGNTASLTITGSQAEDEADYYCSSRDSSDNH
VVFGGGTKLTVL
(SEQ ID NO: 26)

TABLE 14
MSLN-WC-R3-TEA2-C9 Ab Variable Region
amino acid sequences

V_{H} chain of TEA2-C9
EVQLVESGGGLVKAGGSLRLSCTASGFKFTDYYMSWIRQTPGKGLEWV
SYINTSSNHINYAHSVKGRFTISRDNAINSLFLQMDRLTSEDTAVYYC
ARGASWGPLWGQGTLVTVSS
(SEQ ID NO: 27)
V_{L} chain of TEA2-C9
QPGLTQPPPVSVAPGQTARITCGANNIGSKSVHWYQQKPGQAPVLVVS
DDSDRPSGIPERFSGSSSGTTATLTITEVHAEDEADYYCQSADYNWLC
DFGGRTNLTVL
(SEQ ID NO: 28)

TABLE 15
MSLN-WC-R3-TEA1-E8 Ab Variable Region
amino acid sequences

V_{H} chain of TEA1-E8
QVQLVQSGAEVKKPGASVKVSCKASGYTFTHYAVHWVRQAPGQGLEWM
GWIHAGNGNSRFSEKFQGRVTITRDTAASTAYMDLSNLKSEDTAVYYC
AREVGHGMDVWGQGTTVTVSS
(SEQ ID NO: 29)
V_{L} chain of TEA1-E8
QSVLTQPPSVSGAPGQRVTISCTGSSSNIGAGYDVHWYQQLPGTAPKL
LIYGNTNRPSGVPDRFSGSKSGTSASLAITGLQAEDGADYYCQSYDSS
LSGYVFGTGTKVTVL
(SEQ ID NO: 30)

The amino acid sequences of the heavy and light chain complementary determining regions of the MSLN antibodies are shown in Table 16A-B below:

TABLE 16A
Heavy chain (V_{H}) complementary determining
regions (CDRs) of the MSLN antibodies

| Sequence ID | V_{H} CDR1 | V_{H} CDR2 | V_{H} CDR3 |
|---|---|---|---|
| Gly3-1-H10 | GYTLTTNG | VSPYNGHI | ARVNRANYYGMDV |
| | (SEQ ID NO: 31) | (SEQ ID NO: 32) | (SEQ ID NO: 33) |
| TEA2-E10 | GYTLTTNG | VSPYNGHI | ARVNRANYYGMDV |
| | (SEQ ID NO: 31) | (SEQ ID NO: 32) | (SEQ ID NO: 33) |
| TEA1-D3 | GYTLTTNG | VNPYNGHI | ARVNRANYYGMDV |
| | (SEQ ID NO: 31) | (SEQ ID NO: 34) | (SEQ ID NO: 33) |
| Gly3-2-C1 | GYTLTTNG | VSPYNGHI | ARVNRANYYGMDV |
| | (SEQ ID NO: 31) | (SEQ ID NO: 32) | (SEQ ID NO: 33) |
| TEA1-D2 | GYTLTTNG | VSPYNGHI | ARVNRANYYGMDV |
| | (SEQ ID NO: 31) | (SEQ ID NO: 32) | (SEQ ID NO: 33) |
| Gly2-2-F7 | GYTLTTNG | VSPYNGHI | ARVNRANYYGMDV |
| | (SEQ ID NO: 31) | (SEQ ID NO: 32) | (SEQ ID NO: 33) |
| Gly1-2-H4 | GGTFSSDT | INPNSGGT | ARESALGGSYPLSF |
| | (SEQ ID NO: 35) | (SEQ ID NO: 36) | (SEQ ID NO: 37) |
| Gly1-2-D4 | GFTFDDYA | ISWNSGSI | AKDPSSSWLAGAFDI |
| | (SEQ ID NO: 38) | (SEQ ID NO: 39) | (SEQ ID NO: 40) |
| TEA1-E5 | GFTFDDYA | ISWNSGSI | AKDPSSSWLAGAFDI |
| | (SEQ ID NO: 38) | (SEQ ID NO: 39) | (SEQ ID NO: 40) |
| Gly3-2-C10 | GFTFDDYA | ISWNNGSI | AKDPSTSWLAGAFDI |
| | (SEQ ID NO: 38) | (SEQ ID NO: 41) | (SEQ ID NO: 42) |
| Gly2-1-F2 | GFTFDDYA | ISWNSGNI | AKDAGSSGYFNAFDI |
| | (SEQ ID NO: 38) | (SEQ ID NO: 43) | (SEQ ID NO: 44) |
| Gly2-2-B6 | GFTFDDYA | ISWNSGNI | AKDAGSSGYFNAFDI |
| | (SEQ ID NO: 38) | (SEQ ID NO: 43) | (SEQ ID NO: 44) |
| Gly1-2-E10 | GFTFDDYA | ISWNSGSI | AKSPSSNWYPDAFDI |
| | (SEQ ID NO: 38) | (SEQ ID NO: 39) | (SEQ ID NO: 45) |
| TEA2-C9 | GFKFTDYY | INTSSNHI | ARGASWGPL |
| | (SEQ ID NO: 46) | (SEQ ID NO: 47) | (SEQ ID NO: 48) |
| TEA1-E8 | GYTFTHYA | IHAGNGNS | AREVGHGMDV |
| | (SEQ ID NO: 49) | (SEQ ID NO: 50) | (SEQ ID NO: 51) |

TABLE 16B
Light chain (V_{L}) complementary determining
regions (CDRs) of the MSLN antibodies

| Sequence ID | V_{L} CDR1 | V_{L} CDR2 | V_{L} CDR3 |
|---|---|---|---|
| Gly3-1-H10 | QSLLRSNGYNY | LGS | MQSSTNSAH |
| | (SEQ ID NO: 52) | (SEQ ID NO: 53) | (SEQ ID NO: 54) |
| TEA2-E10 | QSLLRSNGYNY | LGS | MQSSTNSAH |
| | (SEQ ID NO: 52) | (SEQ ID NO: 53) | (SEQ ID NO: 54) |
| TEA1-D3 | QSLLRSNGYNY | LGS | MQALQTPLT |
| | (SEQ ID NO: 52) | (SEQ ID NO: 53) | (SEQ ID NO: 55) |
| Gly3-2-C1 | QSLLRSNGYNY | LGS | MQALQTPL |
| | (SEQ ID NO: 52) | (SEQ ID NO: 53) | (SEQ ID NO: 56) |
| TEA1-D2 | QSLLRSNGYNY | LGS | MQALQTPLT |
| | (SEQ ID NO: 52) | (SEQ ID NO: 53) | (SEQ ID NO: 57) |
| Gly2-2-F7 | QSLLRSNGYNY | LGS | HASSTNSAH |
| | (SEQ ID NO: 52) | (SEQ ID NO: 53) | (SEQ ID NO: 58) |
| Gly1-2-H4 | QSVSSSY | GAS | LQDDSYPLT |
| | (SEQ ID NO: 59) | (SEQ ID NO: 60) | (SEQ ID NO: 61) |
| Gly1-2-D4 | SLRRFY | GKN | NSRDSDGNHVF |
| | (SEQ ID NO: 62) | (SEQ ID NO: 63) | (SEQ ID NO: 64) |
| TEA1-E5 | SLRRFY | GKN | NSRDSDGNHVF |
| | (SEQ ID NO: 62) | (SEQ ID NO: 63) | (SEQ ID NO: 64) |
| Gly3-2-C10 | SLIRFY | GKN | NSQDRDGNHVF |
| | (SEQ ID NO: 65) | (SEQ ID NO: 63) | (SEQ ID NO: 66) |
| Gly2-1-F2 | SLRAYY | GKN | NSRDSSGNHLGV |
| | (SEQ ID NO: 67) | (SEQ ID NO: 63) | (SEQ ID NO: 68) |
| Gly2-2-B6 | SLRAYY | GKN | NSRDSSGNHLGS |
| | (SEQ ID NO: 67) | (SEQ ID NO: 63) | (SEQ ID NO: 69) |
| Gly1-2-E10 | SLRNYY | AKN | SSRDSSDNHVV |
| | (SEQ ID NO: 70) | (SEQ ID NO: 71) | (SEQ ID NO: 72) |
| TEA2-C9 | NIGSKS | DDS | QSADYNWLCD |
| | (SEQ ID NO: 73) | (SEQ ID NO: 74) | (SEQ ID NO: 75) |
| TEA1-E8 | SSNIGAGYD | GNT | QSYDSSLSGYV |
| | (SEQ ID NO: 76) | (SEQ ID NO: 77) | (SEQ ID NO: 78) |

The amino acid sequences of the heavy and light chain framework regions of the MSLN antibodies are shown in Table 17A-B below:

TABLE 17A
Heavy chain (V_{H}) framework regions
(FRs) of the MSLN antibodies

| Sequence ID | VH FR1 | VH FR2 | VH FR3 | VH FR4 |
|---|---|---|---|---|
| Gly3-1-H10 | QVQLVQSGAEV | LSWVRQAPGHGL | SYARNLEGRITMT | WGQGTTVTVSS |
| | KKPGASVRVSCK | EWMGW | TDTSTNTAHMELR | (SEQ ID NO: 82) |
| | AS | (SEQ ID NO: 80) | RLTSDDTAVYYC | |
| | (SEQ ID NO: 79) | | (SEQ ID NO: 81) | |
| TEA2-E10 | QVQLVQSGAEV | LSWVRQAPGHGL | SYARNLEGRITMT | WGQGTTVTVSS |
| | KKPGASVRVSCK | EWMGW | TDTSTNTAHMELR | (SEQ ID NO: 82) |
| | AS | (SEQ ID NO: 80) | RLTSDDTAVYYC | |
| | (SEQ ID NO: 79) | | (SEQ ID NO: 81) | |
| TEA1-D3 | QVQLVQSGPEV | LTWVRQAPGHGL | NYARNLKGRITMT | WGQGTTVTVSS |
| | KKPGASLRVSCK | EWMGW | TDTSTNTAHMELR | (SEQ ID NO: 82) |
| | AS | (SEQ ID NO: 84) | RLTSDDTAVYYC | |
| | (SEQ ID NO: 83) | | (SEQ ID NO: 85) | |
| Gly3-2-C1 | QVQLVQSGAEV | LSWVRQAPGHGL | SYARNLEGRITMT | WGQGTTVTVSS |
| | KKPGASVRVSCK | EWMGW | TDTSTNTAHMELR | (SEQ ID NO: 82) |
| | AS | (SEQ ID NO: 80) | RLTSDDTAVYYC | |
| | (SEQ ID NO: 79) | | (SEQ ID NO: 86) | |
| TEA1-D2 | QVQLVQSGAEV | LSWVRQAPGHGL | SYARNLEGRITMT | WGQGTTVTVSS |
| | KKPGASVRVSCK | EWMGW | TDTSTNTAHMELR | (SEQ ID NO: 82) |
| | AS | (SEQ ID NO: 80) | RLTSDDTAVYYC | |
| | (SEQ ID NO: 79) | | (SEQ ID NO: 86) | |
| Gly2-2-F7 | QVQLVQSGAEV | LSWVRQAPGHGL | SYARNLEGRITMT | WGQGTTVTVSS |
| | KKPGASVRVSCK | EWMGW | TDTSTNTAHMELR | (SEQ ID NO: 82) |
| | AS | (SEQ ID NO: 80) | RLTSDDTAVYYC | |
| | (SEQ ID NO: 79) | | (SEQ ID NO: 86) | |
| Gly1-2-H4 | QVQLVQSGAEV | INWVRQAPGQGL | NYAQKFQGWVT | WGQGTLVTVSS |
| | KKPGSSVKVSCK | EWMGW | MTRDTSISTAYME | (SEQ ID NO: 90) |
| | SS | (SEQ ID NO: 88) | LSRLRSDDTAVYYC | |
| | (SEQ ID NO: 87) | | (SEQ ID NO: 89) | |
| Gly1-2-D4 | EVQLVQSGGGL | MHWVRQAPGKG | GYADSVKGRFTISR | WGQGTMVTVSS |
| | VQPGRSLRLSCA | LEWVSG | DNAKNSLYLQMN | (SEQ ID NO: 94) |
| | AS | (SEQ ID NO: 92) | SLRAEDTALYYC | |
| | (SEQ ID NO: 91) | | (SEQ ID NO: 93) | |
| TEA1-E5 | EVQLVQSGGGL | MHWVRQAPGKG | GYADSVKGRFTISR | WGQGTMVTVSS |
| | VQPGRSLRLSCA | LEWVSG | DNAKNSLYLQMN | (SEQ ID NO: 94) |
| | AS | (SEQ ID NO: 92) | SLRAEDTALYYC | |
| | (SEQ ID NO: 91) | | (SEQ ID NO: 93) | |
| Gly3-2-C10 | EVQLVQSGGGLI | MHWVRQAPGKG | GYADSVKGRFPISR | WGQGTMVTVSS |
| | QPGGSLRLSCAA | LEWMSW | NNPQNSLYLQMN | (SEQ ID NO: 94) |
| | S | (SEQ ID NO: 96) | SLKAEDTALYYC | |
| | (SEQ ID NO: 95) | | (SEQ ID NO: 97) | |
| Gly2-1-F2 | EVQLVQSGGGL | MHWVRQAPGKG | GYADSVKGRFTISR | WGQGTMVTVSS |
| | VQPGRSLRLSCA | LEWVSG | DNAKNSLYLQMN | (SEQ ID NO: 94) |
| | AS | (SEQ ID NO: 92) | SLRAEDTALYYC | |
| | (SEQ ID NO: 91) | | (SEQ ID NO: 98) | |
| Gly2-2-B6 | EVQLVQSGGGL | MHWVRQAPGKG | GYADSVKGRFTISR | WGQGTMVTVSS |
| | VQPGRSLRLSCA | LEWVSG | DNAKNSLYLQMN | (SEQ ID NO: 94) |
| | AS | (SEQ ID NO: 92) | SLRAEDTALYYC | |
| | (SEQ ID NO: 91) | | (SEQ ID NO: 98) | |
| Gly1-2-E10 | QVQLVQSGGGL | MHWVRQGPGKG | GFADSVKGRFTISR | WGQGTMVTVSS |
| | VQPGRSLRLSCA | LEWVSG | DNAKNSLYLQMN | (SEQ ID NO: 94) |
| | AS | (SEQ ID NO: 100) | SLRVEDTALYYC | |
| | (SEQ ID NO: 99) | | (SEQ ID NO: 101) | |
| TEA2-C9 | EVQLVESGGGL | MSWIRQTPGKGL | NYAHSVKGRFTISR | WGQGTLVTVSS |
| | VKAGGSLRLSCT | EWVSY | DNAINSLFLQMDR | (SEQ ID NO: 105) |
| | AS | (SEQ ID NO: 103) | LTSEDTAVYYC | |
| | (SEQ ID NO: 102) | | (SEQ ID NO: 104) | |
| TEA1-E8 | QVQLVQSGAEV | VHWVRQAPGQGL | RFSEKFQGRVTITR | WGQGTTVTVSS |
| | KKPGASVKVSCK | EWMGW | DTAASTAYMDLSN | (SEQ ID NO: 109) |
| | AS | (SEQ ID NO: 107) | LKSEDTAVYYC | |
| | (SEQ ID NO: 106 | | (SEQ ID NO: 108) | |

TABLE 17B
Light chain (V_{L}) framework regions
(FRs) of the MSLN antibodies

| Sequence ID | VL FR1 | VL FR2 | VL FR3 | VL FR4 |
|---|---|---|---|---|
| Gly3-1-H10 | EIVLTQSPLSLPVT | LDWYLQKPGQSP | NRASGVPDRFSAS | FGGGTKVEIK |
| | PGEPASISCRSS | QLLIY | GSGTDFTLKISRVE | (SEQ ID NO: 113) |
| | (SEQ ID NO: | (SEQ ID NO: | AEDVGVYYC | |
| | 110) | 111) | (SEQ ID NO: 112) | |
| TEA2-E10 | EIVLTQSPLSLPVT | LDWYLQKPGQSP | NRASGVPDRFSAS | FGGGTKVEIK |
| | PGEPASISCRSS | QLLIY | GSGTDFTLKISRVE | (SEQ ID NO: 113) |
| | (SEQ ID NO: | (SEQ ID NO: | AEDVGVYYC | |
| | 110) | 111) | (SEQ ID NO: 112) | |
| TEA1-D3 | EIVLTHSPLSLPVT | LDWYLHNPGQSP | IRASGVPDRFSAS | FSGGTNVEIK |
| | PGKPASISCRSS | QLLIY | GSRTDFTLKISKED | (SEQ ID NO: 117) |
| | (SEQ ID NO: | (SEQ ID NO: | ADDVGVYYC | |
| | 114) | 115) | (SEQ ID NO: 116) | |
| Gly3-2-C1 | EIVLTQSPLSLPVT | LDWYLQKPGQSP | NRASGVPDRFSAS | FRGGTKVEIK |
| | PGEPASISCRSS | QLLIY | GSCTDFTLKISRVE | (SEQ ID NO: 119) |
| | (SEQ ID NO: | (SEQ ID NO: | AEDVGVYYC | |
| | 110) | 111) | (SEQ ID NO: 118) | |
| TEA1-D2 | EIVLTQSPLSLPVT | LDWYLQKPGQSP | NRASGVPDRFSAS | FGGGTKVEIK |
| | PGEPASISCRSS | QLLIY | GSGTDFTLKISRVE | (SEQ ID NO: 113) |
| | (SEQ ID NO: | (SEQ ID NO: | AEDVGVYYC | |
| | 110) | 111) | (SEQ ID NO: 112) | |
| Gly2-2-F7 | EIVLTQSPLSLPVT | LDWYLQKPGQSP | NRASGVPDRFSAS | FGGGTKVEIK |
| | PGEPASISCRSS | QLLIY | GSGTDFTLKISRVE | (SEQ ID NO: 113) |
| | (SEQ ID NO: | (SEQ ID NO: | AEDVGGLL | |
| | 110) | 111) | (SEQ ID NO: 120) | |
| Gly1-2-H4 | ETTLTQSPGTLSLS | LAWYQQKPGQA | SRATGIPDRFSGS | FGQGTKVEIK |
| | PGERATLSCRAS | PRLLIY | GSGTDFTLTISRLE | (SEQ ID NO: 124) |
| | (SEQ ID NO: | (SEQ ID NO: | PEDSATYYC | |
| | 121) | 122) | (SEQ ID NO: 123) | |
| Gly1-2-D4 | SSELTQDPTVSVA | ASWYQQKPGQA | NRPSGIPDRFSGSY | FGGGTKLTVL |
| | LGQTVRITCQGD | PILVIY | SGNTASLTITGAQ | (SEQ ID NO: 128) |
| | (SEQ ID NO: | (SEQ ID NO: | AEDEADYYC | |
| | 125) | 126) | (SEQ ID NO: 127) | |
| TEA1-E5 | SSELTQDPTVSVA | ASWYQQKPGQA | NRPSGIPDRFSGSY | FGGGTKLTVL |
| | LGQTVRITCQGD | PILVIY | SGNTASLTITGAQ | (SEQ ID NO: 128) |
| | (SEQ ID NO: | (SEQ ID NO: | AEDEADYYC | |
| | 125) | 129) | (SEQ ID NO: 127) | |
| Gly3-2-C10 | SSDLTLDPTVSVA | ASWYHQEPGPAP | TRPSRIPDLFSGSY | FGGGTKLTVL |
| | LGHTVRITCQGD | ILVIY | SRNTSSLTISGAQV | (SEQ ID NO: 128) |
| | (SEQ ID NO: | (SEQ ID NO: | KDEADYYC | |
| | 130) | 131) | (SEQ ID NO: 132) | |
| Gly2-1-F2 | SSELTQDPAVSVA | ASWYQQKPGQA | SRPSGIPDRFSGSN | FGGGTKLTVL |
| | LGQTVRITCQGD | PVLVIY | SGNAASLTITGAQ | (SEQ ID NO: 128) |
| | (SEQ ID NO: | (SEQ ID NO: | AEDEADYYC | |
| | 133) | 134) | (SEQ ID NO: 135) | |
| Gly2-2-B6 | SSELTQDPAVSVA | ASWYQQKPGQA | SRPSGIPDRFSGSN | FGGGTKLTVL |
| | LGQTVRITCQGD | PVLVIY | SGNAASLTITGAQ | (SEQ ID NO: 128) |
| | (SEQ ID NO: | (SEQ ID NO: | AEDEADYYC | |
| | 133) | 134) | (SEQ ID NO: 135) | |
| Gly1-2-E10 | SSELTQDPAVSVA | ASWYQQKPGQA | NRPSGIPDRFSGSS | FGGGTKLTVL |
| | LGQTVRITCQGD | PVLVIY | SGNTASLTITGSQA | (SEQ ID NO: 128) |
| | (SEQ ID NO: | (SEQ ID NO: | EDEADYYC | |
| | 133) | 134) | (SEQ ID NO: 136) | |
| TEA2-C9 | QPGLTQPPPVSV | VHWYQQKPGQA | DRPSGIPERFSGSS | FGGRTNLTVL |
| | APGQTARITCGAN | PVLVVS | SGTTATLTITEVHA | (SEQ ID NO: 140) |
| | (SEQ ID NO: | (SEQ ID NO: | EDEADYYC | |
| | 137) | 138) | (SEQ ID NO: 139) | |
| TEA1-E8 | QSVLTQPPSVSG | VHWYQQLPGTA | NRPSGVPDRFSGS | FGTGTKVTVL |
| | APGQRVTISCTGS | PKLLIY | KSGTSASLAITGLQ | (SEQ ID NO: 144) |
| | (SEQ ID NO: | (SEQ ID NO: | AEDGADYYC | |
| | 141) | 142) | (SEQ ID NO: 143) | |

The MSLN antibodies described herein bind to MSLN. In one embodiment, the MSLN antibodies have high affinity and high specificity for MSLN. Some embodiments also feature antibodies that have a specified percentage identity or similarity to the amino acid or nucleotide sequences of the anti-MSLN antibodies described herein. For example, “homology” or “identity” or “similarity” refers to sequence similarity between two peptides or between two nucleic acid molecules. Homology can be determined by comparing a position in each sequence, which can be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-MSLN antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-MSLN antibodies described herein. Sequence identity or similarity to the nucleic acids and proteins of the invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e., BLAST or BLAST 2.0), manual alignment or visual inspection can be utilized to determine percent sequence identity or similarity for the nucleic acids and proteins of the invention.

“Polypeptide” as used herein can encompass a singular “polypeptide” as well as plural “polypeptides,” and can refer to a molecule composed of monomers (amino acids) linearly linked by amide bonds (also known as peptide bonds). The term “polypeptide” can refer to any chain or chains of two or more amino acids and does not refer to a specific length of the product. Thus, peptides, dipeptides, tripeptides, oligopeptides, “protein,” “amino acid chain,” or any other term can be used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the anti-MSLN antibodies disclosed herein can exhibit increased cross-reactivity to MSLN in comparison to an unmodified MSLN antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

### Antibodies

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the antigen and does not react with other polypeptides.

The terms “antibody fragment” or “antigen-binding fragment”, as used herein, is a portion of an antibody such as F(ab′) 2, F (ab) 2, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′) 2, Fd, Fvs, single-chain Fvs (scFv), single-chain antibodies, dAb (domain antibody), minibodies, disulfide-linked Fvs (sdFv), fragments comprising a VL or VH domain, fragments produced by a Fab expression library, and anti-idiotypic (anti-Id) antibodies.

A “single-chain variable fragment” or “scFv” refers to a fusion protein of the variable regions of the heavy (VH) and light chains (VL) of immunoglobulins. A single chain Fv (“scFv”) polypeptide molecule is a covalently linked VH: VL heterodimer, which can be expressed from a gene fusion including VH- and VL-encoding genes linked by a peptide-encoding linker. (See Huston et al. (1988) Proc Nat Acad Sci USA 85 (16): 5879-5883). In some aspects, the regions are connected with a short linker peptide of ten to about 25 amino acids. The linker can be rich in glycine for flexibility, as well as serine or threonine for solubility, and can connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgAQ1 and IgA2) or subclass of an immunoglobulin molecule.

Light chains are classified as kappa or lambda (K, 2). Each heavy chain class can be bound with a kappa or lambda light chain. In general, the light and heavy chains are covalently bonded to each other, and the “tail” portions of the two heavy chains are bonded to each other by covalent disulfide linkages or non-covalent linkages when the immunoglobulins are generated by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and VL regions, which contain the CDRs, as well as frameworks (FRs) of the MSLN antibodies are shown in Table 1-Table 17.

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include such meanings unless explicitly stated to the contrary. A specific example is the use of the term “complementarity determining region” (“CDR”) to describe the non-contiguous antigen combining sites found within the variable region of both heavy and light chain polypeptides. This region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a certain CDR will vary depending on the sequence and size of the CDR. Those skilled in the art can routinely determine which residues comprise a certain CDR given the variable region amino acid sequence of the antibody.

| CDR | Kabat Numbering | Chothia Numbering | IMGT Numbering |
|---|---|---|---|
| VH CDR1 | 31-35 | 26-32 | 27-38 |
| VH CDR2 | 50-65 | 52-58 | 56-65 |
| VH CDR3 | 95-102 | 95-102 | 105-117 |
| VL CDR1 | 24-34 | 26-32 | 27-38 |
| VL CDR2 | 50-56 | 50-52 | 56-65 |
| VL CDR3 | 89-97 | 91-96 | 105-117 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

In certain aspects, the CDRs of an antibody can be determined according to the IMGT numbering system. The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommié, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3Dstructure-DB [Kaas, Q., Ruiz, M. and Lefranc, M.-P., T cell receptor and MHC structural data. Nucl. Acids. Res., 32, D208-D210 (2004)].

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3). In one embodiment, the antibodies can be directed to mesothelin (MSLN) encoded by NCBI Reference No: NM_005823 and having amino acid sequence:

(SEQ ID NO: 145)
MALPTARPLLGSCGTPALGSLLFLLFSLGWVQPSRTLAGETGQEAAPLD
GVLANPPNISSLSPRQLLGFPCAEVSGLSTERVRELAVALAQKNVKLST
EQLRCLAHRLSEPPEDLDALPLDLLLFLNPDAFSGPQACTRFFSRITKA
NVDLLPRGAPERQRLLPAALACWGVRGSLLSEADVRALGGLACDLPGRF
VAESAEVLLPRLVSCPGPLDQDQQEAARAALQGGGPPYGPPSTWSVSTM
DALRGLLPVLGQPIIRSIPQGIVAAWRQRSSRDPSWRQPERTILRPRFR
REVEKTACPSGKKAREIDESLIFYKKWELEACVDAALLATQMDRVNAIP
FTYEQLDVLKHKLDELYPQGYPESVIQHLGYLFLKMSPEDIRKWNVTSL
ETLKALLEVNKGHEMSPQVATLIDRFVKGRGQLDKDTLDTLTAFYPGYL
CSLSPEELSSVPPSSIWAVRPQDLDTCDPRQLDVLYPKARLAFQNMNGS
EYFVKIQSFLGGAPTEDLKALSQQNVSMDLATFMKLRTDAVLPLTVAEV
QKLLGPHVEGLKAEERHRPVRDWILRQRQDDLDTLGLGLQGGIPNGYLV
LDLSMQEALSGTPCLLGPGPVLTVLALLLASTLA

See, for example, product data sheet for SC110135 (Mesothelin (MSLN) (NM_005823) Human Untagged Clone.

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows for the cancellation of parameters not related to affinity, and is equal to the equilibrium binding constant, K_{D}. (See, generally, Davies et al. (1990) Annual Rev Biochem 59:439-473). An antibody of the invention can specifically bind to a MSLN epitope when the equilibrium binding constant (K_{D}) is ≤1 μM, ≤10 μM, ≤10 nM, ≤10 μM, or ≤100 μM to about 1 μM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it can bind to a random, unrelated epitope.

For example, the MSLN antibody can be monovalent or bivalent, and/or can comprise a single or double chain. Functionally, the binding affinity of the MSLN antibody is within the range of 10⁻⁵M to 10⁻¹² M. For example, the binding affinity of the MSLN antibody is from 10⁻⁶ M to 10⁻¹² M, from 10⁻⁷M to 10⁻¹² M, from 10⁻⁸M to 10⁻¹² M, from 10⁻⁹M to 10⁻¹² M, from 10⁻⁵M to 10⁻¹¹ M, from 10⁻⁶ M to 10⁻¹¹ M, from 10⁻⁷M to 10⁻¹¹ M, from 10⁻⁸ M to 10⁻¹¹ M, from 10⁻⁹M to 10⁻¹¹ M, from 10⁻¹⁰ M to 10⁻¹¹M, from 10⁻⁵M to 10⁻¹⁰M, from 10⁻⁶ M to 10⁻¹⁰ M, from 10⁻⁷M to 10⁻¹⁰ M, from 10⁻⁸ M to 10⁻¹⁰M, from 10⁻⁹ M to 10⁻¹⁰ M, from 10⁻⁵ M to 10⁻⁹ M, from 10⁻⁶ M to 10⁻⁹M, from 10⁻⁷M to 10⁻⁹M, from 10⁻⁸ M to 10⁻⁹M, from 10⁻⁵ M to 10⁻⁸ M, from 10⁻⁶ M to 10⁻⁸ M, from 10⁻⁷M to 10⁻⁸ M, from 10⁻⁵M to 10⁻⁷M, from 10⁻⁶ M to 10⁻⁷M, or from 10⁻⁵M to 10⁻⁶ M.

A MSLN protein, or a derivative, fragment, analog, homolog or ortholog thereof, can be utilized as an immunogen in the generation of antibodies that immunospecifically bind these protein components. A MSLN protein or a derivative, fragment, analog, homolog, or ortholog thereof, coupled to a proteoliposome can be utilized as an immunogen in the generation of antibodies that immunospecifically bind these protein components.

Those skilled in the art will recognize that one can determine, without undue experimentation, if a human monoclonal antibody has the same specificity as a human monoclonal antibody of the invention by ascertaining whether the former prevents the latter from binding to MSLN. For example, if the human monoclonal antibody being tested competes with the human monoclonal antibody of the invention, as shown by a decrease in binding by the human monoclonal antibody of the invention, then the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with the MSLN protein, with which it is normally reactive, and then add the human monoclonal antibody being tested to determine if the human monoclonal antibody being tested is inhibited in its ability to bind MSLN. If the human monoclonal antibody being tested is inhibited, then it can have the same, or functionally equivalent, epitopic specificity as the monoclonal antibody of the invention. Screening of human monoclonal antibodies of the invention can be also carried out by utilizing MSLN and determining whether the test monoclonal antibody is able to neutralize MSLN.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. The complementarity determining regions (CDRs) of the monoclonal antibody are identical in the molecules of the population. MAbs contain an antigen binding site that can immunoreact with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that produce or can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are preferred, or spleen cells or lymph node cells can be used if non-human mammalian sources are preferred. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, such as myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas can include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, California) and the American Type Culture Collection (Manassas, Virginia). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that can bind specifically to genes encoding the heavy and light chains of murine antibodies). The hybridoma cells of the invention serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, and/or can be substituted for the variable domains of one antigen-combining site of an antibody of the invention to create a chimeric bivalent antibody.

Fully human antibodies, for example, are antibody molecules in which the entire sequence of both the light chain and the heavy chain, including the CDRs, arise from human genes. Such antibodies are termed “human antibodies” or “fully human antibodies”. Human monoclonal antibodies, such as fully human and humanized antibodies, can be prepared by using trioma technique; the human B-cell hybridoma technique (see Kozbor, et al, 1983 Immunol Today 4:72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

“Humanized antibodies” can be antibodies from a non-human species (such as mouse), whose amino acid sequences (for example, in the CDR regions) have been modified to increase their similarity to antibody variants produced in humans. Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant can be a tobacco plant, i.e., Nicotiana benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes the antibody of the invention or produces the antibody of the invention.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5): 489-498 (1991); Studnicka et al., Protein Engineering 7 (6): 805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (such as rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20).

In addition, antibodies (such as human antibodies) can also be produced using other techniques, including phage display libraries. (See Hoogenboom and Winter, J. Mol. Biol, 227:381 (1991); Marks et al., J. Mol. Biol, 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al., Nature 368 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13:65-93 (1995).

Human antibodies can additionally be produced using transgenic nonhuman animals which are modified to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides the preferred modifications is then obtained as progeny by crossbreeding intermediate transgenic animals containing fewer than the full complement of the modifications. A non-limiting example of such a nonhuman animal is a mouse and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publication nos. WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described herein. Vectors include, but are not limited to, chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used and include moloney murine leukemia viruses. DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87:1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G . . . et al, Nat. Genet. 8:148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The vector chosen will depend upon the target cell and the condition being treated. The introduction can be by standard techniques, e.g., infection, transfection, transduction or transformation. Examples of modes of gene transfer include e.g., naked DNA, CaP0₄ precipitation, DEAE dextran, electroporation, protoplast fusion, lipofection, cell microinjection, and viral vectors.

The vector can be employed to target essentially any target cell. For example, stereotaxic injection can be used to direct the vectors (e.g., adenovirus, HSV) to a preferred location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of MSLN in a sample. The antibody can also be used to try to bind to and disrupt a MSLN activity.

In an embodiment, the antibodies described herein can be full-length antibodies, including those containing an Fc region similar to wild-type Fc regions that bind to Fc receptors.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246:1275-1281) to allow rapid and effective identification of monoclonal Fab fragments with the preferred specificity for a protein or derivatives, fragments, analogs or homologs thereof. Antibody fragments that contain the idiotypes to a protein antigen can be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Heteroconjugate antibodies are also within the scope of the invention. Heteroconjugate antibodies are composed of two covalently joined antibodies. Such antibodies can, for example, target immune system cells to unwanted cells (see U.S. Pat. No. 4,676,980), and for treatment of HIV infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176:1 191-1 195 (1992) and Shopes, J. Immunol., 148:2918-2922 (1992)). Alternatively, an antibody can be engineered that has dual Fc regions and can thereby have enhanced complement lysis and ADCC capabilities. (See Stevenson et al, Anti-Cancer Drug Design, 3:219-230 (1989)). In one embodiment, the antibody of the invention has modifications of the Fc region, such that the Fc region does not bind to the Fc receptors. For example, the Fc receptor is Fcγ receptor. Antibodies with modification of the Fc region such that the Fc region does not bind to Fcγ, but still binds to neonatal Fc receptor are useful as described herein.

In embodiments, an antibody of the invention can comprise an Fc variant. See, for example, WO2018/145075 and WO2019/183362, which provide Fc variant compositions for augmenting antibody mediated receptor signaling. In embodiments, the Fc variant can comprise an amino acid substitution which alters the antigen-independent effector functions of the antibody, such as the circulating half-life of the antibody. Such antibodies exhibit increased or decreased binding to FcRn when compared to antibodies lacking these substitutions, therefore, have an increased or decreased half-life in serum, respectively. Fc variants with improved affinity for FcRn can have longer serum half-lives, and such molecules have useful applications in methods of treating mammals where long half-life of the administered antibody is preferred, e.g., to treat a chronic disease or disorder. In contrast, Fc variants with decreased FcRn binding affinity can have shorter half-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be preferred include those applications in which localization to the brain, kidney, and/or liver is preferred. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In an embodiment, the antibody comprises an Fc variant with an amino acid substitution at amino acid position 228 or 299 (EU numbering). In an embodiment, the antibody comprises an IgG1 or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g., “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG4 antibody which is devoid of Fc-effector function thereby eliminating the potential for Fc mediated toxicity to the normal vital tissues and cells that express MSLN. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, 90Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of possible moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved by direct condensation of existing side chains or by the incorporation of external bridging molecules. Many bivalent or polyvalent linking agents are useful in coupling protein molecules, such as the antibodies of the invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al, Science 238:1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS(-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82:3688 (1985); Hwang et al, Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the preferred diameter. Fab′ fragments of the antibody of the invention can be conjugated to the liposomes as described in Martin et al, J. Biol. Chem., 257:286-288 (1982) via a disulfide-interchange reaction.

### Multispecific Antibodies (Bispecific and Trispecific)

Embodiments as described herein can comprise a monospecific antibody or a multispecific antibody.

Monospecific antibodies are antibodies with one or more binding sites that specifically binds to a single antigen.

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. This invention provides for multispecific antibodies, such as bi-specific and trispecific antibodies, that recognize MSLN and a second antigen and/or a third antigen. In one embodiment, multispecific antibodies (e.g., bi-specific antibodies and trispecific antibodies) can comprise MSLN specific fusion proteins encompassing the antibodies described herein. Exemplary second and or third antigens include tumor associated antigens (e.g., LINGO1), cytokines (e.g., IL-12 (IL-12A (p35 subunit) protein sequence having NCBI Reference No. NP_000873.2; IL-12B (p40 subunit) protein sequence having NCBI Reference No. NP_002178.2); IL-18 (protein sequence having NCBI Reference no. NP_001553.1); IL-15 (protein sequence having NCBI Reference No. NP_000576.1); IL-7 (protein sequence having NCBI Reference No. NP_000871.1); IL-2 (protein sequence having NCBI Reference No. NP_000577.2); and IL-21 (protein sequence having NCBI Reference No. NP_068575.1)), cytokine cognate receptors (eg., IL-12R), and cell surface receptors. Non-limiting examples of second and/or third antigens include CTLA-4, CXCR4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM (or BTLA), CD47 and CD73. In one embodiment, the bispecific and trispecific antibodies comprise MSLN fusion proteins. For example, the fusion protein comprises an antibody comprising a variable domain or scFv unit and a ligand or antigen and/or a third ligand or antigen as described herein such that the resulting antibody recognizes an antigen and binds to the ligand-specific receptor. Exemplary antibody compositions (e.g., VH and/or VL sequences or fragments thereof) that are useful for the design of MSLN fusion proteins as described herein include, but are not limited to, anti-CAIX antibodies described in PCT/US2006/046350 and PCT/US2015/067178; anti-CXCR4 antibodies described in PCT/US20006/005691 and PCT/US2019/022272; anti-CCR4 antibodies described in PCT/US2008/088435, PCT/US2013/039744, and PCT/US2015/054202; anti-PD-L1 antibodies described in PCT/US2008/088435 and PCT/US2020/062815; anti-PD-1 antibodies described in PCT/US2020/037791 and PCT/US2020/037781; anti-GITR antibodies described in PCT/US2017/043504; anti-claudin-4 antibodies described in PCT/US2019/022272; and anti-MUC1 antibodies described in PCT/US2020/037783 (each of the applications which are incorporated by reference in their entireties). In one embodiment, the fusion protein further comprises a constant region, and/or a linker as described herein. Different formats of multispecific antibodies (e.g., bispecific antibodies and trispecific antibodies such as a fusion protein comprises an antibody that recognizes MSLN and a ligand) are described herein. Ligands can be tumor associated antigens (e.g., LINGO1, ErbB2 (HER2/neu), carcinoembryonic antigen (CEA), epithelial cell adhesion molecule (EpCAM), epidermal growth factor receptor (EGFR), MUC1, MSLN, CD19, CD20, CD30, CD40, CD22, RAGE-1, MN-CA IX, RET1, RET2 (AS), prostate specific antigen (PSA), TAG-72, PAP, p53, Ras, prostein, PSMA, survivin, 9D7, prostate-carcinoma tumor antigen-1 (PCTA-1), GAGE, MAGE, mesothelin, β-catenin, TGF-βRII, BRCA1/2, SAP-1, HPV-E6, HPV-E7 (see also, PCT/US2015/067225 and PCT/US2019/022272 for additional tumor-associated surface antigens, which are incorporated by reference in their entireties)); cytokines (e.g., IL-12 (IL-12A (p35 subunit) protein sequence having NCBI Reference No. NP_000873.2; IL-12B (p40 subunit) protein sequence having NCBI Reference No. NP_002178.2); IL-18 (protein sequence having NCBI Reference no. NP_001553.1); IL-15 (protein sequence having NCBI Reference No. NP_000576.1); IL-7 (protein sequence having NCBI Reference No. NP_000871.1); IL-2 (protein sequence having NCBI Reference No. NP_000577.2); and IL-21 (protein sequence having NCBI Reference No. NP_068575.1)); CTLA-4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM (or BTLA), CD47 and CD73. Different formats of bispecific or trispecific antibodies are also provided herein. In some embodiments, each of the anti-MSLN fragment and the second antigen-specific fragment and/or the third antigen-specific fragment is each independently selected from a Fab fragment, a single-chain variable fragment (scFv), or a single-domain antibody. In some embodiments, the bispecific or trispecific antibody further includes a Fc fragment (e.g., as described in PCT/US2015/021529 and PCT/US2019/023382, each of which are incorporated by reference in their entireties). A bispecific or trispecific antibody of the invention can comprise a heavy chain and a light chain combination or scFv of the MSLN antibodies described herein.

Multispecific antibodies (e.g., bispecific antibodies and trispecific antibodies) of the invention (for example, an anti-MSLN-scFv fusion protein) can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker depicted herein (GGGGSGGGGS; “(G₄S)₂”) can be generated with a longer G4S linker to improve flexibility. For example, the linker can also be:
“(G₄S)₃” (e.g., GGGGGGGGSGGGGS);
“(G₄S)₄” (e.g., GGGGSGGGGSGGGGSGGGGS);
“(G₄S)₅” (e.g., GGGGSGGGGSGGGGSGGGGSGGGGS);
“(G₄S)₆” (e.g., GGGGSGGGGSGGGGSGGGGSGGGGSGGGGS);
“(G₄S)₇” (e.g., GGGGGGGGSGGGGSGGGGSGGGGSGGGGSGGGGS); and the like. For example, use of the (G4S) s linker can provide more flexibility to a ligand described herein and can improve expression. In some embodiments, the linker can also be (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. Non-limiting examples of linkers known to those skilled in the art that can be used to construct the fusions described herein can be found in U.S. Pat. No. 9,708,412; U.S. Patent Application Publication Nos. US20180134789 and US20200148771; and PCT Publication No. WO2019051122 (each of which are incorporated by reference in their entireties).

In another embodiment, the multispecific antibodies (e.g., bispecific antibodies and trispecific antibodies such as anti-MSLN-scFv fusions) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens/ligands or three different antigens/ligands are mixed to promote heteroligation pairing, which is mediated through the engineered “knob into holes” of the CH3 domains.

In another embodiment the multispecific antibodies (e.g., bispecific antibodies and trispecific antibodies such as anti-MSLN-scFv fusions) can be constructed through exchange of heavy-light chain dimers from two or more different antibodies to generate a hybrid antibody where the first heavy-light chain dimer recognizes MSLN and the second heavy-light chain dimer recognizes a second antigen and/or third antigen. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by inter heavy chain disulfide bridges in the hinge region of the antibody. Specifically, in IgG₄, the hinge region contains the amino acid sequence Cys-Pro-Ser-Cys (in comparison to the stable IgG₁ hinge region which contains the sequence Cys-Pro-Pro-Cys) at amino acids 226-230. This sequence difference of Serine at position 229 has been linked to the tendency of IgG₄ to form intrachain disulfides in the hinge region (Van der Neut Kolfschoten, M. et al, 2007, Science 317:1554-1557 and Labrijn, A. F. et al, 2011, Journal of Immunol 187:3238-3246).

The multispecific antibodies (e.g., bispecific antibodies and trispecific antibodies such as anti-MSLN-scFv fusions) of the invention can be created through introduction of the R409 residue in the CH3 domain and the Cys-Pro-Ser-Cys sequence in the hinge region of antibodies that recognize MSLN or a second and/or third antigen, so that the heavy-light chain dimers exchange to produce an antibody molecule with one heavy-light chain dimer recognizing MSLN and the second heavy-light chain dimer recognizing a second and/or third antigen, wherein the second and/or third antigen (or ligand) is any antigen (or ligand) disclosed herein. Known IgG₄ molecules can also be altered such that the heavy and light chains recognize MSLN or a second and/or third antigen, as disclosed herein. Use of this method for constructing the multispecific antibodies (e.g., bispecific antibodies and trispecific antibodies such as anti-MSLN-scFv fusions) of the invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG₄-based multispecific antibodies (e.g., bispecific antibodies and trispecific antibodies such as anti-MSLN-scFv fusions) attractive for therapeutic applications, in which the antibody is required to bind the target(s) and functionally alter the signaling pathways associated with the target(s), however not trigger effector activities.

The multispecific antibodies (e.g., bispecific antibodies and trispecific antibodies such as anti-MSLN-scFv fusions) described herein can be engineered with a non-depleting heavy chain isotype, such as IgG1-LALA or stabilized IgG4 or one of the other non-depleting variants. In some embodiments, mutations are introduced to the constant regions of the bsAb such that the antibody dependent cell-mediated cytotoxicity (ADCC) activity of the bsAb is altered. For example, the mutation is a LALA mutation in the CH2 domain. In one aspect, the multispecific antibody (e.g., bispecific antibodies and trispecific antibodies such as anti-MSLN-scFv fusions) contains mutations on one scFv unit of the heterodimeric multispecific antibody, which reduces the ADCC activity. In another aspect, the multispecific antibody (e.g., bispecific antibodies and trispecific antibodies such as anti-MSLN-scFv fusions) contains mutations on both chains of the heterodimeric multispecific antibody, which completely ablates the ADCC activity. For example, the mutations introduced in one or both scFv units of the multispecific antibody (e.g., bispecific antibodies and trispecific antibodies such as anti-MSLN-scFv fusions) are LALA mutations in the CH2 domain. These multispecific antibodies (e.g., bispecific antibodies and trispecific antibodies such as anti-MSLN-scFv fusions) with variable ADCC activity can be optimized such that the multi-specific antibodies exhibit maximal selective killing towards cells that express one antigen that is recognized by the multispecific antibody; however, exhibits minimal killing towards the second antigen that is recognized by the multispecific antibody.

The multispecific antibodies (e.g., bispecific antibodies) described herein can be engineered as modular tetrameric bispecific antibodies (tBsAb). See, for example, WO 2018/071913, which is incorporated by reference herein in its entirety. For example, the tetravalent antibody can be a dimer of a bispecific scFv fragment including a first binding site for a first antigen, and a second binding site for a second antigen. In embodiments, the anti-MSLN antibody can be the first binding site for a first antigen. In embodiments, the anti-MSLN antibody can be the second binding site for a second antigen. The two binding sites can be joined together via a linker domain. In embodiments, the scFv fragment is a tandem scFv, the linker domain includes an immunoglobulin hinge region (e.g., an IgG1, an IgG2, an IgG3, or an IgG4 hinge region) amino acid sequence. In embodiments, the immunoglobulin hinge region amino acid sequence can be flanked by a flexible linker amino acid sequence, e.g., having the linker amino acid sequence (GGGS)ₓ₁₋₆, (GGGGS)ₓ₁₋₆, or GSAGSAAGSGEF. In embodiments, the linker domain includes at least a portion of an immunoglobulin Fc domain, e.g., an IgG1, an IgG2, an IgG3, or an IgG4 Fc domain. In embodiments, the at least a portion of the immunoglobulin Fc domain does not include a CH2 domain. In embodiments, the at least a portion of the immunoglobulin Fc domain can be a CH2 domain. An exemplary CH2 domain amino acid sequence includes APELLGGPDVFLF (SEQ ID NO: [ ]). The Fc domain can be linked to the C-terminus of an immunoglobulin hinge region (e.g., an IgG1, an IgG2, an IgG3, or an IgG4 hinge region) amino acid sequence. The linker domain can include a flexible linker amino acid sequence (e.g., (GGGS)ₓ₁₋₆, (GGGGS)ₓ₁₋₆, or GSAGSAAGSGEF) at one terminus or at both termini.

In embodiments, the (BsAb can be specific for MSLN, and also a target selected from the group consisting of B7H3, CXCR4, B7H4, CD27, CD28, CD40, CD40L, CD47, CD122, CCR4, CTLA-4, GITR, GITRL, ICOS, ICOSL, LAG-3, LIGHT, OX-40, OX40L, PD-L1, PD-1, TIM3, 4-1BB, TIGIT, VISTA, HEVM, BTLA, and KIR.

In embodiments, the multispecific antibody can be a bi-specific T-cell engager (BiTE). The term “BiTEs” (a bispecific T-cell engager) can refer to a single polypeptide chain molecule with two antigen binding domains, one of which binds to a T-cell antigen. For example, the BiTE can comprise a MSLN antibody disclosed herein, or a functional fragment thereof, and an antibody or fragment thereof that binds to a T-cell antigen. For example, the antibody or fragment thereof that binds to a T-cell antigen can be specific for CD3.

In embodiments, the multispecific antibody can be a tri-specific T-cell engager (TriTE). The term “TriTEs” (a trispecific T-cell engager) can refer to a single polypeptide chain molecule with three antigen binding domains, one or more of which binds to a T-cell antigen. For example, the TriTE can comprise a MSLN antibody disclosed herein, or a functional fragment thereof, and an antibody or fragment thereof that binds to a T-cell antigen.

For example, the antibody or fragment thereof that binds to a T-cell antigen can be specific for CD3, CD28, or both.

The multispecific antibodies (e.g., bispecific antibodies and trispecific antibodies such as anti-MSLN-scFv fusions) disclosed herein can be useful in treatment of chronic infections, diseases, or medical conditions, for example, cancer.

### Fusion Proteins

The invention provides a fusion protein containing a MSLN antibody disclosed herein, or a functional fragment thereof, operably linked to a second protein. The second protein can be, for example, a cytokine or a growth factor. In embodiments, the cytokine is IL-2 or TGF-beta and variants thereof. In some other embodiments, the second protein can be a therapeutic agent, such as a toxin, a detectable moiety, such as a fluorescent protein for detection, or a biological agent, such as an agent that stimulates T cells (i.e., CD3). In some embodiments, the MSLN antibodies of the invention can be operably linked to more than one additional protein or peptide, for example 2, 3, 4, 5, 6, 7, 8, 9, or 10 additional proteins or peptide sequences.

In some embodiments, the MSLN antibody disclosed herein, or functional fragment thereof, is joined directly to the second protein. In other embodiments, the MSLN antibody, or functional fragment thereof, is joined to the second protein via a linker, such as a flexible polypeptide chain. The linker can be any suitable linker of any length, but can be at least 1, 2, 3, 4, 5, 10, 15, 20, 25, or 30 amino acids in length. In one embodiment, the linker is an amino acid sequence that is naturally present in immunoglobulin molecules of the host, such that the presence of the linker cannot result in an immune response against the linker sequence by the mammal. Fusion proteins of the invention that include more than one additional protein to the MSLN antibody can have multiple linker sequences that join each additional protein or peptide sequence.

The fusion proteins of the invention can be constructed by recombinant methods known to the skilled artisan. For example, an expression vector containing the nucleic acid sequence encoding a MSLN antibody of the invention can be operably linked to the nucleic acid sequence encoding the second protein and can be introduced to an expression system to translate and produce the fusion protein. Alternatively, one skilled in the art can readily utilize de nova protein synthesis techniques to produce the fusion proteins described herein.

### Use of Antibodies Against MSLN

Antibodies of the invention specifically binding a MSLN protein, or a fragment thereof, can be administered for the treatment of a MSLN associated disease or disorder. A “MSLN-associated disease or disorder” includes disease states and/or symptoms associated with a disease state, where increased levels of MSLN and/or activation of cellular signaling pathways involving MSLN are found. Exemplary MSLN-associated diseases or disorders include, but are not limited to, diseases where T cells are suppressed, such as in cancer and infectious diseases. In some embodiments, the cancer can be lung cancer, kidney cancer, ovarian cancer, prostate cancer, colon cancer, breast cancer, cervical cancer, uterine cancer, brain cancer, skin cancer, liver cancer, pancreatic cancer, or stomach cancer. In some embodiments, the infectious disease is a viral disease, such as influenza.

Antibodies of the invention, including bi-specific, polyclonal, monoclonal, humanized and fully human antibodies, can be used as therapeutic agents. Such agents can be employed to treat cancer in a subject, increase vaccine efficiency or augment a natural immune response. An antibody preparation, for example, one having high specificity and high affinity for its target antigen, is administered to the subject and can have an effect due to its binding with the target. Administration of the antibody can abrogate or inhibit or interfere with an activity of the MSLN protein.

### Pharmaceutical Compositions

Antibodies of the invention specifically binding a MSLN protein or fragment thereof can be administered for the treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for any patient will depend upon a variety of factors, including the antibodies, variant or derivative thereof used, the patient's age, body weight, general health, sex, and diet, and the time of administration, rate of excretion, drug combination, and the severity of the disease being treated. Judgment of such factors by medical caregivers is within the ordinary skill in the art. The amount will also depend on the individual patient to be treated, the route of administration, the type of formulation, the characteristics of the compound used, the severity of the disease, and the preferred effect. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen and will also depend on the rate at which an administered antibody is depleted from the free volume other subject to which it is administered. The dosage administered to a subject (e.g., a patient) of the antigen-binding polypeptides described herein is about 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure can be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90:7889-7893 (1993)). The formulation can also contain more than one active compound as necessary for the indication being treated, for example, those with complementary activities that do not adversely affect each other. Alternatively, or in addition, the composition can comprise an agent that enhances its function, such as, for example, a cytotoxic agent, cytokine (e.g., IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allow for release of molecules for over 100 days, certain hydrogels release proteins for shorter time periods.

The antibodies or agents of the invention (also referred to herein as “active compounds”), and derivatives, fragments, analogs and homologs thereof, can be incorporated into pharmaceutical compositions suitable for administration. Such pharmaceutical compositions can comprise the antibody or agent and a pharmaceutically acceptable carrier. As used herein, the term “pharmaceutically acceptable carrier” can include solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional preferred ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the preferred therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals.

The pharmaceutical compositions can be included in a container, pack, or dispenser together with instructions for administration.

### Diagnostics

An antibody according to the invention can be used as an agent for detecting the presence of MSLN (or a protein fragment thereof) in a sample. For example, the sample can be a cancer sample or a sample from a subject at risk of having cancer. For example, the cancer can be lung cancer, kidney cancer, ovarian cancer, prostate cancer, colon cancer, breast cancer, cervical cancer, uterine cancer, brain cancer, skin cancer, liver cancer, pancreatic cancer, or stomach cancer. For example, the antibody can contain a detectable label. Antibodies can be polyclonal or monoclonal. An intact antibody, or a fragment thereof (e.g., F_{ab}, ScFv, or F_{(ab)2}) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies directed against a MSLN protein (or a fragment thereof) can be used in methods known within the art relating to the localization and/or quantitation of a MSLN protein (e.g., for use in measuring levels of the MSLN protein within appropriate physiological samples, for use in diagnostic methods, for use in imaging the protein, and the like). In a given embodiment, antibodies specific to a MSLN protein, or derivative, fragment, analog or homolog thereof, that contain the antibody derived antigen binding domain, are utilized as pharmacologically active compounds (referred to herein as “therapeutics”).

An antibody of the invention specific for a MSLN protein can be used to isolate a MSLN polypeptide by standard techniques, such as immunoaffinity, chromatography or immunoprecipitation. Antibodies directed against a MSLN protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

### Screening Methods

The invention provides methods (also referred to herein as “screening assays”) for identifying modulators, i.e., candidate or test compounds or agents (e.g., peptides, peptidomimetics, small molecules or other drugs) that modulate or otherwise interfere with a MSLN activity. Also provided are methods of identifying compounds useful to treat cancer. The invention also encompasses compounds identified using the screening assays described herein.

For example, the invention provides assays for screening candidate or test compounds which modulate MSLN expression and/or activity. The test compounds of the invention can be obtained using any of the numerous approaches in combinatorial library methods known in the art, including but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. (See, e.g., Lam, 1997. Anticancer Drug Design 12:145).

A “small molecule” as used herein, can refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90:6909; Erb, et al., 1994. Proc. Natl. Acad. Sci. U.S.A. 91:11422; Zuckermann, et al., 1994. J. Med. Chem. 37:2678; Cho, et al., 1993. Science 261:1303; Carrell, et al., 1994. Angew. Chem. Int. Ed. Engl. 33:2059; Carell, et al., 1994. Angew. Chem. Int. Ed. Engl. 33:2061; and Gallop, et al., 1994. J. Med. Chem. 37:1233.

Libraries of compounds can be presented in solution (see e.g., Houghten, 1992. Biotechniques 13:412-421), or on beads (see Lam, 1991. Nature 354:82-84), on chips (see Fodor, 1993. Nature 364:555-556), bacteria (see U.S. Pat. No. 5,223,409), spores (see U.S. U.S. Pat. No. 5,233,409), plasmids (see Cull, et al., 1992. Proc. Natl. Acad. Sci. USA 89:1865-1869) or on phage (see Scott and Smith, 1990. Science 249:386-390; Devlin, 1990. Science 249:404-406; Cwirla, et al., 1990. Proc. Natl. Acad. Sci. U.S.A. 87:6378-6382; Felici, 1991. J. Mol. Biol. 222:301-310; and U.S. Pat. No. 5,233,409.).

In one embodiment, a candidate compound is introduced to an antibody-antigen complex and determining whether the candidate compound disrupts the antibody-antigen complex, wherein a disruption of this complex indicates that the candidate compound modulates an MSLN activity.

In another embodiment, at least one MSLN protein is provided, which is exposed to at least one monoclonal antibody. Formation of an antibody-antigen complex is detected, and one or more candidate compounds are introduced to the complex. If the antibody-antigen complex is disrupted following introduction of the one or more candidate compounds, the candidate compound is useful to treat cancer or a proliferative disease or disorder.

Determining the ability of the test compound to interfere with or disrupt the antibody-antigen complex can be accomplished, for example, by coupling the test compound with a radioisotope or enzymatic label such that binding of the test compound to the antigen or biologically active portion thereof can be determined by detecting the labeled compound in a complex. For example, test compounds can be labeled with 125r, 35 S, 14C, or 3H, directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In one embodiment, the assay comprises contacting an antibody-antigen complex with a test compound and determining the ability of the test compound to interact with the antigen or otherwise disrupt the existing antibody-antigen complex. In this embodiment, determining the ability of the test compound to interact with the antigen and/or disrupt the antibody-antigen complex comprises determining the ability of the test compound to preferentially bind to the antigen or a biologically active portion thereof, as compared to the antibody.

In another embodiment, the assay comprises contacting an antibody-antigen complex with a test compound and determining the ability of the test compound to modulate the antibody-antigen complex. Determining the ability of the test compound to modulate the antibody-antigen complex can be accomplished, for example, by determining the ability of the antigen to bind to or interact with the antibody, in the presence of the test compound.

Those skilled in the art will recognize that, in any of the screening methods disclosed herein, the antibody can be a MSLN antibody. Additionally, the antigen can be a MSLN protein or a portion thereof.

The screening methods disclosed herein can be performed as a cell-based assay or as a cell-free assay. In the case of cell-free assays comprising the membrane-bound forms of the MSLN proteins, it can be desirable to utilize a solubilizing agent such that the membrane-bound form of the proteins is maintained in solution. Examples of such solubilizing agents include non-ionic detergents such as n-octylglucoside, n-dodecy 1glucoside, n-dodecy 1maltoside, octanoy 1-N-methy 1glucamide, decanoyl-N-methylglucamide, Triton® X-100, Triton® X-114, Thesit®, Isotridecypoly(ethylene glycol ether)n, N-dodecy 1-N,N-dimethy 1-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment, it can be desirable to immobilize the antibody or the antigen to facilitate separation of complexed from uncomplexed forms of one or both following introduction of the candidate compound, as well as to accommodate automation of the assay. Observation of the antibody-antigen complex in the presence and absence of a candidate compound can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-antibody fusion proteins or GST-antigen fusion proteins can be adsorbed onto glutathione sepharose beads (Sigma Chemical, St. Louis, MO) or glutathione derivatized microtiter plates, that are then combined with the test compound, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined directly or indirectly. Alternatively, the complexes can be dissociated from the matrix, and the level of antibody-antigen complex formation can be determined using standard techniques.

Other techniques for immobilizing proteins on matrices can also be used in the screening assays of the invention. For example, the antibody or the antigen (e.g., the MSLN protein) can be immobilized utilizing conjugation of biotin and streptavidin. Biotinylated antibody or antigen molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, other antibodies reactive with the antibody or antigen of interest, but which do not interfere with the formation of the antibody-antigen complex of interest, can be derivatized to the wells of the plate, and unbound antibody or antigen trapped in the wells by antibody conjugation. Methods for detecting such complexes, in addition to those described herein for the GST-immobilized complexes, include immunodetection of complexes using such other antibodies reactive with the antibody or antigen.

The invention further pertains to new agents identified by any of the screening assays described herein and uses thereof for treatments as described herein.

### Chimeric Antigen Receptor (CAR) Cell Therapies

Cellular therapies, such as chimeric antigen receptor (CAR) cell therapies, are also provided herein. For example, the cell can be a CAR T-cell or a CAR NK-cell,

CAR cell therapies redirect a patient's T-cells and/or NK-cells to kill tumor cells by the exogenous expression of a CAR on a T-cell or NK-cell, for example. A CAR can be a membrane spanning fusion protein that links the antigen recognition domain of an antibody to the intracellular signaling domains of the T-cell receptor and co-receptor or NK-cell receptor.

In one embodiment, monospecific CAR cells are provided. For example, the anti-MSLN antibodies described herein can be used as the targeting moiety for the CAR cell. For example, the MSLN antibody can have low affinity but high avidity for its antigen. In another example, the MSLN antibody can have high affinity but low avidity for its antigen. Antibodies with fewer binding sites can have high affinity and low avidity, while those with greater binding sites can have low affinity and high avidity.

In another embodiment, bispecific (or dual-targeted) CAR cells are provided. In another embodiment, the CAR cell is an engineered cell comprising a chimeric antigen receptor, wherein the chimeric antigen receptor comprises an extracellular ligand binding domain that is specific for a first antigen and a second antigen on the surface of a cancer cell, wherein the first antigen comprises an antigen that is not MSLN and the second antigen comprises MSLN.

In embodiments, the anti-MSLN antibodies or the MSLN fusion proteins described herein can be used as a payload for armored CAR-cell therapies. A suitable cell can be used, for example, that can secrete an anti-MSLN antibody of the invention (or alternatively engineered to express an anti-MSLN antibody as described herein to be secreted). The anti-MSLN “payloads” to be secreted, can be, for example, minibodies, scFvs, IgG molecules, bispecific fusion molecules, and other antibody fragments as described herein. Upon contact or engineering, the cell described herein can then be introduced to a patient in need of a treatment by infusion therapies known to one of skill in the art.

In embodiments, the patient can have a MSLN-associated disease or disorder as described herein, such as cancer. The cell (e.g., a T cell) can be, for instance, T lymphocyte, a CD4+ T cell, a CD8+ T cell, or the combination thereof, without limitation. Exemplary CARs and CAR factories useful in aspects of the invention include those disclosed in, for example, PCT/US2015/067225 and PCT/US2019/022272, each of which are hereby incorporated by reference in their entireties. In one embodiment, the MSLN antibodies discussed herein can be used in the construction of multi-specific antibodies or as the payload for a CAR-T cell or CAR NK-cell. For example, in one embodiment, the anti-MSLN antibodies discussed herein can be used for the targeting of the CARs (i.e., as the targeting moiety). In another embodiment, the anti-MSLN antibodies discussed herein can be used as the targeting moiety, and a different MSLN antibody that targets a different epitope can be used as the payload. In another embodiment, the payload can be an immunomodulatory antibody payload.

Solid tumors offer unique challenges for CAR-T therapies. Some barriers to CAR-T effectiveness in solid tumors include heterogeneous antigen expression, insufficient tissue homing, activation, persistence, and the immunosuppressive tumor microenvironment. Unlike blood cancers, tumor-associated target proteins are overexpressed between the tumor and healthy tissue resulting in on-target/off-tumor T-cell killing of healthy tissues. Furthermore, immune repression in the tumor microenvironment (TME) limits the activation of CAR-T cells towards killing the tumor. Upon such contact or engineering, the cell can then be introduced to a cancer patient in need of a treatment by infusion therapies known to one of skill in the art. The cancer patient can have a cancer of any of the types as disclosed herein. The cell (e.g., a T cell) can be, for instance, a tumor-infiltrating T lymphocyte, a CD4+ T cell, a CD8+ T cell, or the combination thereof, without limitation.

In embodiments, CAR cells (i.e., CAR T cells or CAR NK cells) can be generated according to methods known in the art using lentivirus systems (via transduction), retrovirus systems (via transfection (electroporation)), and transposon systems (via PiggyBac). Useful for promoters for payloads that can be used in the generating of CAR-Ts include, for example, constitutive promoters (where the promoter is the same as for CAR-T, such as EF1a then IRES or 2A); inducible promoters (where the promoter is different from the promoter for CAR-T, such as NFAT, IL-2 prom); and genetically engineered promoters (such as a MSLN locus “knock in” of cytokine and/or a promoter that is under the control of an endogenous promoter). In one embodiment, the MSLN antibodies or the MSLN fusion proteins discussed herein can be used in the construction of multi-specific antibodies or as the payload for a CAR cell. For example, in one embodiment, the anti-MSLN antibodies or the MSLN fusion proteins discussed herein can be used for the targeting of the CARS (i.e., as the targeting moiety). In one embodiment, the anti-MSLN antibodies or the MSLN fusion proteins discussed herein can be used as a payload to be secreted by a CAR cell. In another embodiment, the anti-MSLN antibodies or the MSLN fusion proteins discussed herein can be used as the targeting moiety, and a different MSLN antibody that targets a different epitope can be used as the payload. In another embodiment, the payload can be an immunomodulatory antibody payload. In some embodiments, the MSLN antibodies or the MSLN fusion proteins as described herein for use in CAR-T compositions are not high-affinity MSLN antibodies (for example, so that the antibody does not bind strongly to its MSLN target). For example, the MSLN antibodies or the MSLN fusion proteins described herein can be used as a payload secreted by the CAR cell, with the two targeting moieties (for example, tumor-associated surface antigens) selected for a specific cancer. Non-limiting examples of a tumor-associated surface antigen include ErbB2 (HER2/neu), carcinoembryonic antigen (CEA), epithelial cell adhesion molecule (EpCAM), epidermal growth factor receptor (EGFR), MUC1, MSLN, CD19, CD20, CD30, CD40, CD22, RAGE-1, MN-CA IX, RET1, RET2 (AS), prostate specific antigen (PSA), TAG-72, PAP, p53, Ras, prostein, PSMA, survivin, 9D7, prostate-carcinoma tumor antigen-1 (PCTA-1), GAGE, MAGE, mesothelin, β-catenin, TGF-βRII, BRCA1/2, SAP-1, HPV-E6, HPV-E7 (see also, PCT/US2015/067225 and PCT/US2019/022272 for additional tumor-associated surface antigens, which are incorporated by reference in their entireties). Exemplary armored CAR-T cells are listed in the table below.

| CART | Payload | Format | Promoter | Publication |
|---|---|---|---|---|
| PSMA | DN-TGFb | | | Molecular Therapy 26: |
| | | | | 1855 (2018) |
| GD2 | cJun | cDNA | | Nature 576: 293(2019) |
| Fibronectin | CD47 | VHH | | Cancer Immunol Res. 8: |
| PD-L1 | PD-L1 | | | 518-529 (2020) |
| | CTLA-4 | | | |
| GPC3 | IL-12 | | | J Immunol 2019; 203: |
| | | | | 198 |
| CD20 | PD-1 | | | Cancer Science. 2019; |
| | | | | 110: 3079 |
| CD19 | PD-1 | | | nature biotechnology |
| Muc16 | | | | 36: 847 (2018) |
| CD19 | IL-18 | | | Cell Reports 23: 2130 |
| Muc16 | | | | (2018) |
| CD19 | IL-12 | fusion | IRES | Scientific REPOrTS 7: |
| Muc16 | | | | 10541 (2017) |
| CD19 | PD-1 | scFv | P2A | Clin Cancer Res 23: |
| | | | | 6982 (2017) |
| CAE | IL-18 | | NFAT | Cell Reports 21: 3205 |
| | IL-12 | | IL-2 | (2017) |
| VEGF2 | IL-12 | | | Clin Cancer Res 18: |
| | | | | 1672 (2012) |

The skilled artisan will recognize that CAR cells can be generated from cellular sources known to the skilled artisan. Non-limiting examples include T cells, NK cells, iPSC-derived cells (e.g., iPSC-derived T-cells and/or iPSC-derived NK cells), peripheral blood cells (e.g., peripheral blood mononuclear cells), cord blood cells, cell lines (e.g., NK92 cell line), human embryonic stem cells (hESCs) and CD34+ hematopoietic progenitor cells (HPCs). See, for example, Lu, Hui, et al. “From CAR-T cells to CAR-NK cells: a developing immunotherapy method for hematological malignancies.” Frontiers in Oncology (2021): 3151.

### Chimeric B-Cell Receptor

A modified B cell receptor called chimeric B cell receptor, such as a B cell receptor containing an antibody or antibody fragment previously selected by high affinity against a specific disease associated antigen, is a powerful new approach against diseases. As B cells serve as professional antigen presenting cells, they can process and present antigens on MHC class II molecules, enhancing immune cell recognition of the tumor and assisting in neoantigen spreading. A key component of immunologic memory, chimeric antibody signaling and secreting (CASS) B cells will simultaneously recruit a wide range of immune cells and reverse tumor infiltrating lymphocyte exhaustion, providing a robust and lifelong surveillance program protecting against tumor metastasis and recurrence. In embodiments, the B cell can include a receptor that is chimeric, non-natural and engineered at least in part by the hand of man. In cases, the engineered chimeric B cell receptor has one, two, three, four, or more components, and in some embodiments the one or more components facilitate targeting or binding of the B cell to one or more antigen-comprising cells.

Aspects of the invention include genetically engineered B cells that are modified to express and bear on its surface a chimeric B cell receptor. In embodiments, the genetically modified B cell can comprise a single chimeric B cell receptor targeting one antigen, such as MSLN, or a single chimeric B cell receptor targeting two or more antigens (e.g., a bi-specific chimeric B cell receptor, or a multispecific chimeric B cell receptor). In some embodiments, the cells comprise a split chimeric B cell receptor, such as two different scFvs expressed on the B cell surface with different co-stimulation domains. Further, some embodiments comprise a fine-tuned chimeric B cell receptor.

In embodiments, the chimeric B cell receptor comprises an extracellular domain, a transmembrane domain, and an intracellular signaling domain; such that the polypeptides assemble together to form a chimeric B cell receptor.

For example, the extracellular ligand-binding domain can be chosen to recognize a ligand, such as MSLN, that acts as a cell surface marker on target cells associated with a disease state. For example, the disease state can be cancer, and the target ligand can be a cancer associated antigen, such as MSLN.

In embodiments, the extracellular ligand-binding domain can comprise an antigen binding domain or antigen recognition domain derived from an antibody against an antigen of the target, such as an anti-MSLN antibody described herein. In embodiments, the extracellular ligand-binding domain can comprise an antibody or fragment thereof described herein.

In an embodiment, the transmembrane domain comprises a stalk region. The stalk region can be derived from all or part of naturally occurring molecules, such as from all or part of the extracellular region of CD8, CD4 or CD28, or from all or part of an antibody constant region (such as CH1, CH2, CH3, or both CH2 and CH3 for an IgG antibody, or CH1, CH2, CH3, CH4, or any combination thereof for an IgM antibody). In other embodiments, the stalk region can be a synthetic sequence that corresponds to a naturally occurring stalk sequence or can be an entirely synthetic stalk sequence. In an embodiment said stalk region is a part of human CD8 alpha chain.

The signal transducing domain or intracellular signaling domain of the chimeric B cell receptor of the invention is responsible for intracellular signaling following the binding of extracellular ligand binding domain to the target resulting in the activation of the immune cell and immune response. In other words, the signal transducing domain is responsible for the activation of at least one of the normal function of the B cell in which the chimeric B cell receptor is expressed. Thus, the term “signal transducing domain” can refer to the portion of a protein which directs the cell to perform a specialized function, such as early activation of Lyn and Syk and late activation of NFAT and NFκB as examples.

The chimeric B cell receptor can comprise native transmembrane and intracellular domains. In native B cells, engagement of the B cell receptor leads to rapid tyrosine phosphorylation of the intracellular domains and calcium ion polarization, resulting in downstream activation of NFAT and NF-kB. Using the NFAT/NF-kB response elements to drive expression of our secreted proteins, we have designed an inducible expression system that will be activated by antigens associated with disease states, such as cancer.

The distinguishing features of appropriate transmembrane polypeptides comprise the ability to be expressed at the surface of an immune cell, such as B cells, and to interact together for directing cellular response of immune cell against a predefined target cell. The different transmembrane polypeptides of the chimeric B cell receptor comprising an extracellular ligand-binding domain and/or a signal transducing domain interact together to take part in signal transduction following the binding with a target ligand and induce an immune response. The transmembrane domain can be derived from a natural or from a synthetic source. The transmembrane domain can be derived from any membrane-bound or transmembrane protein.

### Methods of Treatment

As used herein, the terms “treat” or “treatment” refer to both therapeutic treatment and prophylactic or preventative measures, wherein the object is to prevent or slow down (lessen) an unsought physiological change or disorder, such as the progression of cancer. Beneficial or preferred clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer (for example, if an early detection cancer biomarker is identified in such a subject), or other cell proliferation-related diseases or disorders. Such diseases or disorders include but are not limited to, e.g., those diseases or disorders associated with aberrant expression of MSLN and/or aberrant activation of cellular signaling pathways involving MSLN. Such diseases or disorders are included in MSLN associated diseases or disorders. For example, the methods are used to treat, prevent, or alleviate a symptom of cancer. In an embodiment, the methods are used to treat, prevent, or alleviate a symptom of a solid tumor. Non-limiting examples of other tumors that can be treated by compositions described herein comprise lung cancer, breast, ovarian cancer, prostate cancer, colon cancer, cervical cancer, brain cancer, skin cancer, liver cancer, pancreatic cancer, or stomach cancer. Additionally, the methods of the invention can be used to treat hematologic cancers such as leukemia and lymphoma. Alternatively, the methods can be used to treat, prevent, or alleviate a symptom of a cancer that has metastasized. For example, cancers that can be treated or prevented or for which symptoms can be alleviated include B-cell chronic lymphocytic leukemia (CLL), non-small-cell lung cancer, melanoma, ovarian cancer, lymphoma, or renal-cell cancer. Cancers that can also be treated or prevented or for which symptoms can be alleviated include those solid tumors with a high mutation burden and WBC in filtrate.

Accordingly, in one aspect, the invention provides methods for preventing, treating, or alleviating a symptom cancer or a cell proliferative disease or disorder in a subject by administering to the subject a monoclonal antibody, scFv antibody or bi-specific antibody of the invention. For example, an anti-MSLN antibody can be administered in therapeutically effective amounts.

Subjects at risk for cancer or cell proliferation-related diseases or disorders can include patients who have a family history of cancer or a subject exposed to a known or suspected cancer-causing agent. Administration of a prophylactic agent can occur prior to the manifestation of cancer such that the disease is prevented or, alternatively, delayed in its progression.

In another aspect, tumor cell growth is inhibited by contacting a cell with an anti-MSLN antibody of the invention. The cell can be any cell that expresses MSLN.

The invention further provides for both prophylactic and therapeutic methods of treating a subject at risk of (or susceptible to) a chronic or acute viral, bacterial, or parasitic infection. The invention also provides for therapeutic methods for both prophylactic and therapeutic methods of treating a subject at risk of a disease or disorder or condition associated with T-cell exhaustion or a risk of developing T-cell exhaustion. The invention also provides for therapeutic methods for both prophylactic and therapeutic methods of treating a subject at risk of a disease or disorder or condition associated with T-cell exhaustion or a risk of developing T-cell exhaustion. Such diseases or disorder include, but are not limited to HIV, AIDS, and chronic or acute bacterial, viral or parasitic infections. Other such chronic infections include those caused by, for example, hepatitis B virus (HBV), hepatitis C virus (HCV), herpes simplex virus 1 (HSV-1), H. pylori, or Toxoplasma gondii. Other acute infections included are those caused by, for example, microorganisms, such as a Gram-positive bacterium, a Gram-negative bacterium, a protozoan, or a fungus, as described herein. A non-limiting example of an infection comprises an influenza infection.

Also included in the invention are methods of increasing or enhancing an immune response to an antigen. An immune response is increased or enhanced by administering to the subject a monoclonal antibody, scFv antibody, or bi-specific antibody of the invention. The immune response is augmented for example by augmenting antigen specific T effector function. The antigen is a viral (e.g., HIV), bacterial, parasitic or tumor antigen. The immune response is a natural immune response. By natural immune response is meant an immune response that is a result of an infection. The infection can be an influenza infection. The infection is a chronic infection. Increasing or enhancing an immune response to an antigen can be measured by a number of methods known in the art. For example, an immune response can be measured by measuring any one of the following: T cell activity, T cell proliferation, T cell activation, production of effector cytokines, and T cell transcriptional profile. Alternatively, the immune response is a response induced due to a vaccination.

Accordingly, in another aspect the invention provides a method of increasing vaccine efficiency by administering to the subject a monoclonal antibody or scFv antibody of the invention and a vaccine. The antibody and the vaccine are administered sequentially or concurrently. The vaccine is a tumor vaccine a bacterial vaccine or a viral vaccine.

### Combinatory Methods

Compositions of the invention as described herein can be administered in combination with a chemotherapeutic agent. Chemotherapeutic agents that can be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In additional embodiments, the compositions of the invention as described herein can be administered in combination with cytokines. Cytokines that can be administered with the compositions include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

The invention provides for methods of treating cancer in a patient by administering two antibodies that bind to the same epitope of the MSLN protein or, alternatively, two different epitopes of the MSLN protein. Alternatively, the cancer can be treated by administering a first antibody that binds to MSLN and a second antibody that binds to a protein other than MSLN. In other embodiments, the cancer can be treated by administering a bispecific antibody that binds to MSLN and that binds to a protein other than MSLN. For example, the other protein other than MSLN can include, but is not limited to, IL-12, IL-12R, IL-2, IL-2R, IL-15, IL-15R, IL-7, IL-7R, IL-21, or IL-21R. For example, the other protein other than MSLN is a tumor-associated antigen; the other protein other than MSLN can also be a cytokine. Non-limiting examples of the other protein other than MSLN includes CTLA-4, CXCR4, LAG-3, CD28, CD122, 4-1BB, TIM3, OX-40, OX40L, CD40, CD40L, LIGHT, ICOS, ICOSL, GITR, GITRL, TIGIT, CD27, VISTA, B7H3, B7H4, HEVM (or BTLA), CD47 and CD73.

In some embodiments, the invention provides for the administration of an anti-PD-1 antibody alone or in combination with an additional antibody that recognizes another protein other than MSLN, with cells that can effect or augment an immune response. For example, these cells can be peripheral blood mononuclear cells (PBMC), or any cell type that is found in PBMC, e.g., cytotoxic T cells, macrophages, and natural killer (NK) cells.

Additionally, the invention provides administration of an antibody that binds to the MSLN protein and an anti-neoplastic agent, such as a small molecule, a growth factor, a cytokine or other therapeutic including biomolecules such as peptides, peptidomimetics, peptoids, polynucleotides, lipid-derived mediators, small biogenic amines, hormones, neuropeptides, and proteases. Small molecules include, but are not limited to, inorganic molecules and small organic molecules. Suitable growth factors or cytokines include an IL-2, GM-CSF, IL-12, and TNF-alpha. Small molecule libraries are known in the art. (See, Lam, Anticancer Drug Des., 12:145, 1997.)

### Diagnostic Assays

The anti-MSLN antibodies can be used diagnostically to, for example, monitor the development or progression of cancer as part of a clinical testing procedure to, e.g., determine the efficacy of a given treatment and/or prevention regimen.

In some embodiments, for diagnostic purposes, the anti-MSLN antibody of the invention is linked to a detectable moiety, for example, so as to provide a method for detecting a cancer cell in a subject at risk of or suffering from a cancer.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al, (Proc Natl Acad Sci USA. 1990 March; 87 (6): 2047-51) describe a method of making a vector construct able to express a fusion protein of aequorin and an antibody gene in mammalian cells.

As used herein, the term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject (such as a biopsy), as well as tissues, cells and fluids present within a subject. That is, the detection method of the invention can be used to detect cells that express MSLN in a biological sample in vitro as well as in vivo. For example, in vitro techniques for detection of MSLN include enzyme linked immunosorbent assays (ELISAs), Western blots, immunoprecipitations, and immunofluorescence. Furthermore, in vivo techniques for detection of MSLN include introducing into a subject a labeled anti-MSLN antibody. For example, the antibody can be labeled with a radioactive marker whose presence and location in a subject can be detected by standard imaging techniques.

In the case of “targeted” conjugates, that is, conjugates which contain a targeting moiet—a molecule or feature designed to localize the conjugate within a subject or animal at a certain site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can simply refer to the location of the entity within the subject or animal at selected time periods after the entity is administered. By way of another example, localization is achieved when an moiety becomes distributed following administration.

A reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used can have a high enough sensitivity to allow for the imaging of faint light from within a mammal in a reasonable amount of time, and to use the signal from such a device to construct an image.

In cases where it is possible to use light-generating moieties which are extremely bright, and/or to detect light-generating fusion proteins localized near the surface of the subject or animal being imaged, a pair of “night-vision” goggles or a standard high-sensitivity video camera, such as a Silicon Intensified Tube (SIT) camera (e.g., from Hammamatsu Photonic Systems, Bridgewater, N.J.), can be used. However, a more sensitive method of light detection is required.

In extremely low light levels the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to simply detect the presence of a signal (photon) and to count the occurrence of the signal with respect to its position over time.

At least two types of photodetector devices, described herein, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately-120° C. “Backthinned” refers to an ultra-thin backplate that reduces the path length that a photon follows to be detected, thereby increasing the quantum efficiency. A sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons to construct an image which can be, for example, displayed on a monitor or printed on a video printer. Such image processors are sold as part of systems which include the sensitive photon-counting cameras described herein, and accordingly, are available from the same sources. The image processors can be connected to a personal computer, such as an IBM-compatible PC or an Apple Macintosh (Apple Computer, Cupertino, Calif), which can or cannot be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. One exemplary biological sample is a peripheral blood leukocyte sample isolated by conventional means from a subject.

The invention also encompasses kits for detecting the presence of MSLN or a TIGIT-expressing cell in a biological sample. For example, the kit can comprise: a labeled compound or agent that can detect a cancer or tumor cell (e.g., an anti-MSLN scFv or monoclonal antibody) in a biological sample; means for determining the amount of MSLN in the sample; and means for comparing the amount of MSLN in the sample with a standard. The standard is, in some embodiments, a non-cancer cell or cell extract thereof. The compound or agent can be packaged in a suitable container. The kit can further comprise instructions for using the kit to detect cancer in a sample.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention is further described in the following examples, which do not limit the scope of the invention described in the claims

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

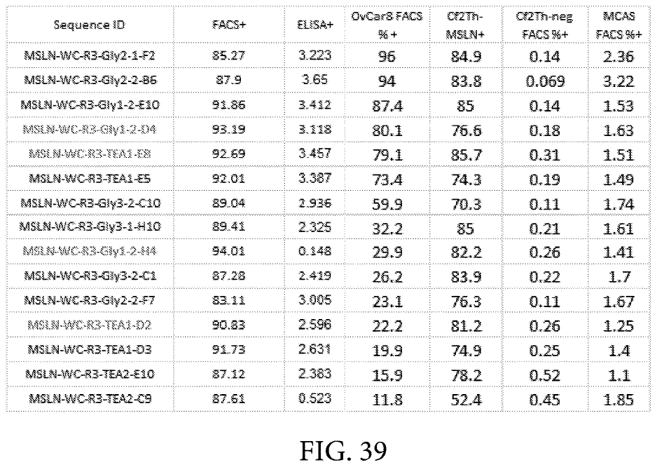

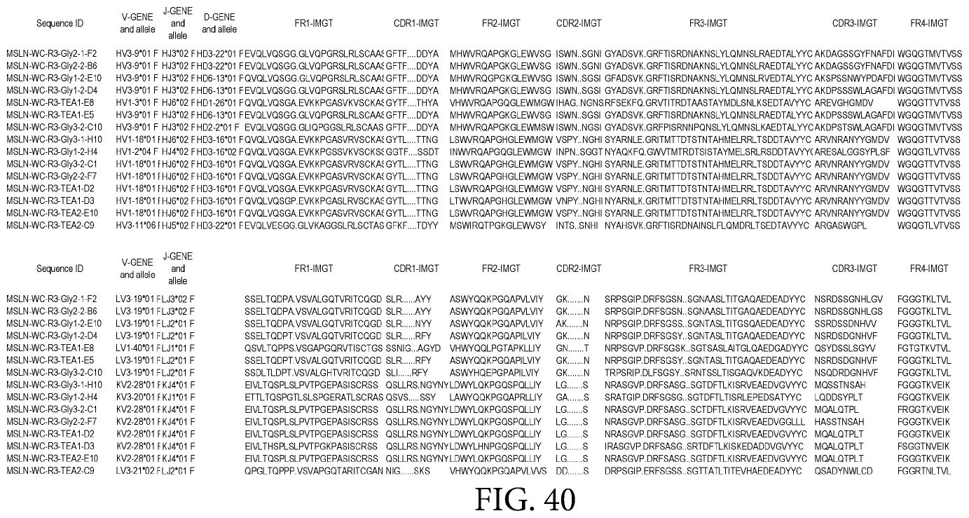

We have identified a series of anti-Mesothelin (MSLN) antibodies via whole cell panning. Panning rounds were completed using transduced cells (293T-MSLN or Cf2Th-MSLN) or tumor cell lines that are naturally high in MLSN (OVCAR8). A total of 118 unique antibodies were identified and the lead clones were identified based on binding to OvCar8 and/or Cf2Th cells. See, FIG. 39. The antibodies were broadly epitope mapped to target the distal region of MSLN, which is also the binding site of MUC16. Additionally, one of our antibodies (Gly1-2-H4) only recognized cell surface expressed MSLN and did not bind to soluble MSLN coated plates, indicating a conformational epitope. A subset of these scFvs were used as a targeting moiety for CAR T cells and displayed potent killing against MSLN+ cells.

MSLN has been identified as a tumor associated antigen for a wide variety of cancers, including lung and ovarian cancers. MSLN can exist in two forms, bound to the cell membrane via a GPI linker, and in a soluble/shed form. The binding of the GPI linker results in a subtle conformational shift in the protein that is recognized by one of our antibodies. By developing a CAR T that can only recognize the membrane bound form of the protein, we can avoid soluble MSLN acting as a decoy and protect our CARS from premature activation.

These scFvs can be developed as monoclonal antibodies for therapeutic or diagnostic purposes. Additionally, they have potential as a targeting moiety for CAR T cells against cancers that overexpress MSLN.

### Example 2

### Chimeric Antibody Signaling and Secreting (CASS) B Cells-A New Combination Cellular Immunotherapy to Achieve Cancer Cures

B cells are a critical component of a patients' natural immunity and while other immune cells including T cells, NK cells, and myeloid cells have been engineered with Chimeric Antigen Receptors (CAR), B cells have largely been ignored. The main role of B cells is to recognize foreign invaders via a membrane bound antibody (B cell receptor, BCR) and eliminate them by producing antibodies that bind and clear the threat. We propose to develop the Chimeric Antibody Signaling and Secreting (CASS) B cell platform by engineering both the target binding domain and secreted antibody to develop a potent anti-cancer therapeutic drug. The target binding domain will be designed to recognize a tumor associated antigen and upon binding will lead to the secretion of a trispecific T cell engaging antibody that effectively stimulates recruited T cells and brings them into contact with tumor cells further enhancing tumor cell lysis. A second major function of B cells is to present foreign protein as MHC-II fragments to T cells and the formation of immune cell clusters, leading to their activation and the further recruitment of various anti-tumor immune cells. Lastly, B cells play a key role in immunologic memory and CASS B cells will provide a lifelong anti-tumor surveillance network protecting patients from metastasis and reoccurrence after the initial tumor is eliminated.

While the CASS B cell platform is applicable to a number of cancers, we will use high grade serous ovarian cancer (HGSOC) as an experimental model. HGSOC was chosen because while it is both the most common and lethal form of ovarian cancer, few new therapies have been developed and the overall survival rate remains poor.

Aim 1 will validate the engineering of the CASS B cell, first by building the synthetic B cell receptor to recognize Muc1. Upon binding to tumor cell expressing MUC1, the CASS B cells will inducibly secrete a trispecific T cell engaging antibody that recognizes a second tumor associated mesothelin (MSLN) (CD3xCD28xMSLN) and further activates T cells to engage in tumor cell recognition.

Aim 2 will use 2D and 3D tissue culture experiments to validate CASS B cell engineering and to demonstrate both the efficacy of the CASS B cell, and the ability of the trispecific antibody to stimulate and engage T cells.

Aim 3 will use humanized mice to validate the effectiveness of the CASS B cell platform in vivo and allow for detailed molecular characterization of the anti-tumor immune response. In embodiments, we can study HGSOC. Without wishing to be bound by theory CASS B cells are a pioneering platform with the potential to provide cures for a diverse range of cancers.

Immune modulating antibodies (Abs) and adoptive cellular therapies have had a transformative effect on cancer therapy, changing the focus from killing tumor cells to activating a patients' natural anti-tumor immunity and reversing the immunosuppressive tumor microenvironment (TME). While these represent some of the most promising anti-cancer therapeutics to date, they often display limited efficacy in high grade serous ovarian cancer (HGSOC) patients¹⁻³. This is in part due to the low mutational burden and limited neoantigen generation seen in HGSOC tumors, resulting in an immunologically “cold” tumor with minimal tumor infiltrating lymphocytes (TILs)^{4,5}. For those immune cells that do reach the tumor site, they are met by a plethora of immunosuppressive cells including myeloid-derived suppressor cells, regulatory T cells, and tumor associated M2 macrophages^{6,7}. To combat this, embodiments herein comprise Chimeric Antibody Secreting and Signaling (CASS) B cells, a new and innovative cellular immunotherapy platform.

As natural antigen presenting cells (APCs), CASS B cells take advantage their ability to present antigens on MHC class II molecules, enhancing tumor cell recognition and neoantigen spreading while also relying on their role in the formation and function of tertiary lymphoid structures (TLS) to secrete chemokines that recruit various immune cell subsets to the HGSOC tumor site^{8,9}. To further improve tumor recognition and engagement by recruited T cells, we will engineer CASS B cells to express a modified tumor associated antigen (TAA) targeted B cell receptor (BCR), which upon activation will induce expression of an anti-CD3xCD28xTAA trispecific T cell engaging antibody locally at the tumor site. For this study, and mucin-1 (Muc1) and mesothelin (MSLN) have been chosen for targeting of the CASS B cell and T cell engager respectively¹⁰⁻¹⁵. The combination of anti-CD3xCD28 on the trispecific antibody will allow for efficient stimulation of both CD4 and CD8 T cells, and the anti-MSLN domain homes the activated T cells towards the tumor, facilitating potent tumor cell lysis¹⁶. While the trispecific antibody developed here is designed to enhance T cell mediated cytotoxicity, it will not directly inhibit the immunosuppressive signals generated in the TME. To block immunosuppression and further enhance efficacy of both the CASS B cell and recruited T cells, we will use a combination therapy using CASS B cells and systemic delivery of an anti-PD1 antibody^{17,18}. With no intrinsic cytotoxic capabilities of their own, the CASS B cell platform is unique in the cell therapy space, as CASS B cells have the power to both initiate a robust anti-tumor response and to augment the activity of the recruited immune cells.

Anti-Muc1 targeted CASS B cells secreting an anti-CD3xCD28xMSLN T cell engaging Ab for HGSOC are described herein. The modular design of CASS B cells allow for easy targeting of other TAAs and the secreted payload can be adjusted to target the relevant immunologic axis, allowing for the adaptation of CASS B cells to a wide variety of cancers. As B cells are long lived and an important part of immunologic memory, CASS B cells continuously deliver the therapeutic payload at the tumor and after the primary tumor has been eradicated, provides a lifelong immunosurveillance system against metastasis and reoccurrence.

Without wishing to be bound by theory, tumor localized activation of CASS B cells will remodel the HGSOC tumor microenvironment via innate antigen presenting capabilities while the formation of tertiary lymphoid structures (TLS) will result in the recruitment and activation of a wide subset of immune cells. Further, CASS B cells engineered to secrete an anti-CD3xCD28xMSLN trispecific T cell engaging antibody will further restore anti-tumor immunity, enhancing T cell activation, tumor recognition, and cytotoxic activity.

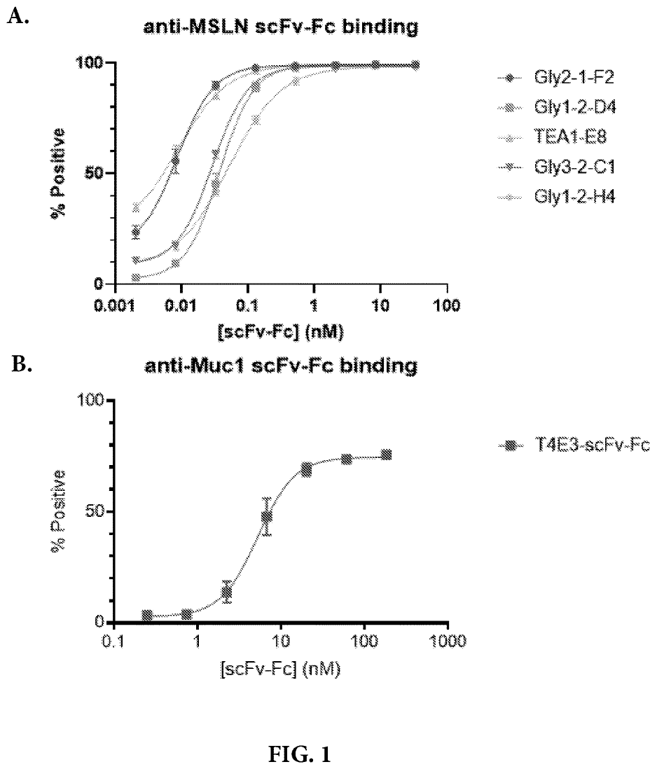

We used a 27-billion member human phage library^{19,20,22,23,25} to identify sets of anti-MSLN (FIG. 1 Top) scFvs and a Muc1 scFv (FIG. 1 Bottom).

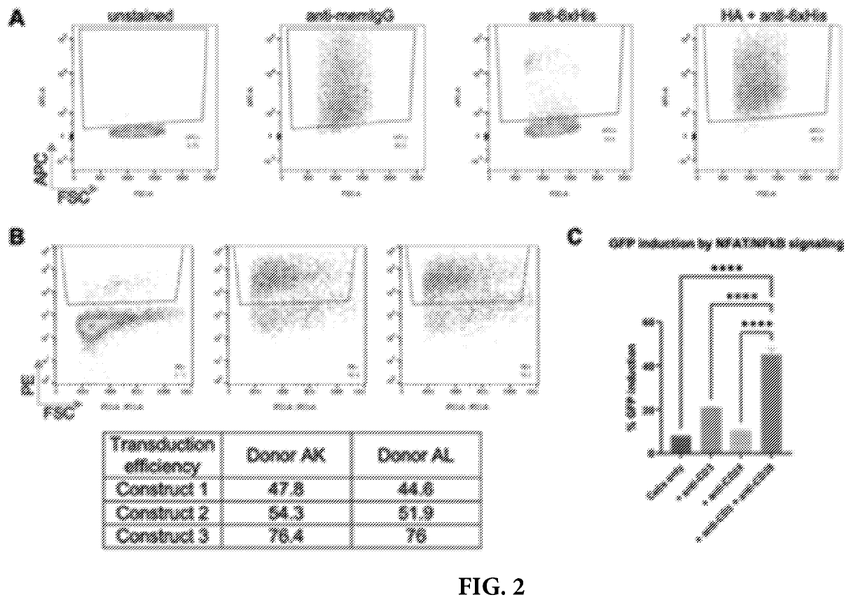

Our engineered IgG-BCR is built using an scFv fused to a membrane bound IgG1 hinge-Fc that expresses at high levels and binds the target antigen (FIG. 2 panel A)²⁶. Transduction experiments using primary B cells demonstrate high titer transduction for multiple donors and DNA constructs (FIG. 2 panel B). We have utilized a NFAT/NFkB response element (RE) to develop an inducible T cell activation assay (FIG. 2 panel C). A fluorescent protein will be used in place of the secreted trispecific T cell engager antibody.

### Non-Limiting Aims

Non-Limiting Aim 1: Engineering and optimization of CASS B cell constructs and trispecific Ab payload—The appropriate Abs (anti-MSLN, Muc1, CD3, CD28) will be selected for CASS B cell development followed by optimization of the signaling domains and inducible response element (RE).

Non-Limiting Aim 2: In vitro evaluation of CASS B and trispecific T cell engager—In vitro characterization will be performed to identify activation thresholds and quantify Ab secretion levels. 3D organoid cultures will be used for an in-depth molecular and functional characterization of both the CASS B cell and the T cell engaging payload.

Non-Limiting Aim 3: In vivo efficacy using HLA matched, humanized HGSOC mouse models—Final evaluation of the optimized CASS B cell construct with and without systemic anti-PD1 combination therapy will be tested in vivo using a cell line derived xenograft (CDX) model in humanized mice.

Non-Limiting Aim 1: Engineering and optimization of CASS B cell constructs and trispecific Ab payload:

Non-Limiting Aim 1A: Antibody discovery and trispecific T cell engager engineering—In this sub aim we will develop the four antibodies; an anti-Muc1 for IgG-BCR targeting and anti-CD3, CD28, MSLN for the trispecific T cell engager. Affinity modulation and epitope mapping will be performed to further characterize these antibodies.

Non-Limiting Aim 1B: CASS B cell vector design and engineering—The CASS B cell lentiviral vector will be designed using an engineered IgG-BCR targeting construct and an NFAT/NFκB inducible reporter system. We will validate the ability of the IgG-BCR to induce expression of a fluorescent protein via the engineered transcription factor response elements (RE), including various NFAT and NFκB formats with minimal promoters²⁷⁻³⁰.

Non-Limiting Aim 1C: Optimization of transduction and exponential expansion of primary B cells—As primary B cells require an array of cytokines and stimulatory signals, we will further engineer a 3T3-msCD40L feeder cell line to express IL-2, 15, and 21 to support maximal B cell growth^{31,32}. We will validate an optimized protocol for the transduction and exponential expansion of primary B cells. Lentiviral transduction of primary B cells will be optimized by testing different envelope proteins (VSVG, BaEV, GaLV) for lentivirus encapsulation and promoters (Ef1α, SFFV) to drive expression of the IgG-BCR³³⁻³⁶.

### Non-Limiting Aim 2: In Vitro Validation of CASS B Cells and the Trispecific T Cell Engager

Non-Limiting Aim 2A: In vitro functional validation of anti-Muc1 CASS B cells—The CASS B cell construct will be assembled using the trispecific T cell engager developed in Non-Limiting Aim 1. Characterization will be performed using soluble, crosslinked Muc1 to determine the CASS B cell threshold of activation and to quantify levels of trispecific Ab expression.

Non-Limiting Aim 2B: In vitro efficacy of CASS B cell in 3D culture utilizing HGSOC tumor spheroids—Muc1/MSLN positive HGSOC cell lines will engrafted in HLA matched, humanized mice. Tumors harvested from these mice will be used with microfluidic chips to generate a 3D organoid model of HGSOC in vitro37-40. This will allow us to validate CASS B cell homing to the tumor spheroid and cytokine analysis of the TME will allow for detailed validation of the effect of the T cell engaging payload. Single cell RNAseq and multiparameter FACS will be used for a detailed molecular and mechanistic interrogation of CASS B cell and T cell engager function and efficacy.

### Non-Limiting Aim 3: In Vivo Efficacy Using HLA Matched, Humanized HGSOC Mouse Models

Non-Limiting Aim 3A: Validate the anti-tumor efficacy and persistence of anti-Muc1 CASS B cells in humanized NSG-SGM3 mice bearing CDX tumors—HLA matched, humanized NSG-SGM3 mice will be used to generate a HGSOC model using a Muc1/MSLN positive HGSOC cell line. These models will then be used to validate efficacy of both the trispecific T cell engager and the CASS B cell as a whole, with various advanced analytical techniques (IHC, multiparameter FACS, scRNAseq) used to further interrogate the effects of CASS B cell therapy on not only the tumor but the immune system as a whole.

Non-Limiting Aim 3B: Validate efficacy of CASS B cells in combination with systemic anti-PD1 therapy—HGSOC CDX models described in Non-Limiting Aim 3A will be used to test CASS B cell efficacy in combination with anti-PD1 therapy. Molecular signatures and phenotypic analysis of immune cell exhaustion markers will be evaluated and compared to those identified in Non-Limiting Aim 3A.

Statistical considerations: With 5 animals per group for individual pair-wise comparisons between conditions of interest and different outcome measures we have power of 0.93 to detect a difference in the means equal to 2.5SD using two-sample two-tailed t-test at 0.05 level. In practice, we will apply linear mixed effects models approach. Bioconductor R packages flowWorkspace will process the flow data and will be visualized using the R package flowFlowJo. Relationships between expression will be examined using Spearman Correlation and mutual information. Moran's Index will be used to quantify spatial autocorrelation of proteins on the segmented RNAscope ISH image, and Gabriel graphs will be used to evaluate spatial patterns correlated to response. Multivariate spatial associations between proteins will be assessed using simple Moran's eigenvector maps and spatial Principal Component Analysis. Additional statistical analysis will be provided by consultation with the Dana-Farber Biostatistics Core.

In embodiments, further engineering can be performed by modification or replacement of the IgG-BCR signaling domains. We have experience with humanized mice and cohorts will be generated assuming a 20% dropout rate due to animal death or lack of CD34 engraftment. Potential alternatives if we are unable to attain suitable humanization with CD34 engraftment is humanization via hPBMC injection. The use of CDX models will allow for easier establishment of our models for both in vitro and in vivo studies.

### REFERENCES CITED IN THIS EXAMPLE

1. Doo, D. W., Norian, L. A. & Arend, R. C. Checkpoint inhibitors in ovarian cancer: A review of preclinical data. Gynecol. Oncol. Reports 29, 48-54 (2019).
2. Nero, C. et al. Patient-derived organoids and high grade serous ovarian cancer: from disease modeling to personalized medicine. J. Exp. Clin. Cancer Res. 40, 1-14 (2021).
3. Matulonis, U. A. et al. Antitumor activity and safety of pembrolizumab in patients with advanced recurrent ovarian cancer: results from the phase II KEYNOTE-100 study. Ann. Oncol. 30, 1080-1087 (2019).
4. Wu, J. W. Y. et al. T-Cell Receptor Therapy in the Treatment of Ovarian Cancer: A Mini Review. Front. Immunol. 12, 1-8 (2021).
5. Bonaventura, P. et al. Cold tumors: A therapeutic challenge for immunotherapy. Front. Immunol. 10, 1-10 (2019).
6. Ciucci, A. et al. Ovarian low and high grade serous carcinomas: Hidden divergent features in the tumor microenvironment. Oncotarget 7, 68033-68043 (2016).
7. Drakes, M. L., Czerlanis, C. M. & Stiff, P. J. Immune checkpoint blockade in gynecologic cancers: State of affairs. Cancers (Basel). 12, 1-20 (2020).
8. Kreiter, S. et al. Mutant MHC class II epitopes drive therapeutic immune responses to cancer. Nature 520, 692-696 (2015).
9. Alspach, E. et al. MHC-II neoantigens shape tumour immunity and response to immunotherapy. Nature 574, 696-701 (2019).
10. Izar, B. et al. A single-cell landscape of high-grade serous ovarian cancer. Nat. Med. 26, 1271-1279 (2020).
11. Wang, L. et al. Expression of MUC1 in primary and metastatic human epithelial ovarian cancer and its therapeutic significance. Gynecol. Oncol. 105, 695-702 (2007).
12. Feng, H., Ghazizadeh, M., Konishi, H. & Araki, T. Expression of MUC1 and MUC2 mucin gene products in human ovarian carcinomas. Jpn. J. Clin. Oncol. 32, 525-529 (2002).
13. Weidemann, S. et al. Mesothelin Expression in Human Tumors: A Tissue Microarray Study on 12,679 Tumors. Biomedicines 9, 4-9 (2021).
14. Hilliard, T. S. The impact of Mesothelin in the Ovarian cancer tumor microenvironment. Cancers (Basel). 10, (2018).
15. Yildiz, Y. et al. High expression of mesothelin in advanced serous ovarian cancer is associated with poor prognosis. J. B.U.ON. 24, 1549-1554 (2019).
16. Wu, L. et al. Trispecific antibodies enhance the therapeutic efficacy of tumor-directed T cells through T cell receptor co-stimulation. Nat. Cancer 1, 86-98 (2020).
17. Waite, J. C. et al. Tumor-targeted CD28 bispecific antibodies enhance the antitumor efficacy of PD-1 immunotherapy. Sci. Transl. Med. 12, (2020).
18. Mullard, A. Trispecific antibodies take to the clinic. Nat. Rev. Drug Discov. 19, 657-658 (2020).
19. Chang, D.-K. et al. Humanization of an anti-CCR4 antibody that kills cutaneous T-cell lymphoma cells and abrogates suppression by T-regulatory cells. Mol. Cancer Ther. 11, 2451-2461 (2012).
20. Xu, C. et al. Unique biological properties of catalytic domain directed human anti-CAIX antibodies discovered through phage-display technology. PLOS One 5, (2010).
21. Chang, D. K. et al. Humanized mouse G6 anti-idiotypic monoclonal antibody has therapeutic potential against IGHV1-69 germline gene-based B-CLL. MAbs 8, 787-798 (2016).
22. Sui, J. et al. Structural and functional bases for broad-spectrum neutralization of avian and human influenza A viruses. Nat. Struct. Mol. Biol. 16, 265-273 (2009).
23. Fu, Y. et al. A broadly neutralizing anti-influenza antibody reveals ongoing capacity of haemagglutinin-specific memory B cells to evolve. Nat. Commun. 7, (2016).
24. Suarez, E. R. et al. Chimeric antigen receptor T cells secreting anti-PD-L1 antibodies more effectively regress renal cell carcinoma in a humanized mouse model. Oncotarget 7, (2016).
25. Chang, D. K. et al. Humanized mouse G6 anti-idiotypic monoclonal antibody has therapeutic potential against IGHV1-69 germline gene-based B-CLL. MAbs 8, 787-798 (2016).
26. Wan, Z. et al. The activation of IgM- or isotype-switched IgG- and IgE-BCR exhibits distinct mechanical force sensitivity and threshold. Elife 4, 1-24 (2015).
27. Uchibori, R. et al. Functional Analysis of an Inducible Promoter Driven by Activation Signals from a Chimeric Antigen Receptor. Mol. Ther.-Oncolytics 12, 16-25 (2019).
28. Zhang, L. et al. Improving adoptive T cell therapy by targeting and controlling IL-12 expression to the tumor environment. Mol. Ther. 19, 751-759 (2011).
29. Hooijberg, E., Bakker, A. Q., Ruizendaal, J. J. & Spits, H. NFAT-controlled expression of GFP permits visualization and isolation of antigen-stimulated primary human T cells. Blood 96, 459-466 (2000).
30. Lin, T. et al. Establishment of NF-κB sensing and interleukin-4 secreting mesenchymal stromal cells as an “on-demand” drug delivery system to modulate inflammation. Cytotherapy 19, 1025-1034 (2017).
31. Whaley, R. E. et al. Generation of a cost-effective cell line for support of high-throughput isolation of primary human B cells and monoclonal neutralizing antibodies. J. Immunol. Methods 488, 112901 (2021).
32. Huang, J. et al. Isolation of human monoclonal antibodies from peripheral blood B cells. Nat. Protoc. 8, 1907-1915 (2013).
33. Girard-Gagnepain, A. et al. Baboon envelope pseudotyped LVs outperform VSV-G-LVs for gene transfer into early-cytokine-stimulated and resting HSCs. Blood 124, 1221-1231 (2014).
34. Tomás, H. A. et al. Improved GaLV-TR Glycoproteins to Pseudotype Lentiviral Vectors: Impact of Viral Protease Activity in the Production of LV Pseudotypes. Mol. Ther.-Methods Clin. Dev. 15, 1-8 (2019).
35. Bernadin, O. et al. Baboon envelope LVs efficiently transduced human adult, fetal, and progenitor T cells and corrected SCID-X1 T-cell deficiency. Blood Adv. 3, 461-475 (2019).
36. Winiarska, M. et al. Selection of an optimal promoter for gene transfer in normal B cells. Mol. Med. Rep. 16, 3041-3048 (2017).
37. Jenkins, R. W. et al. Ex vivo profiling of PD-1 blockade using organotypic tumor spheroids. Cancer Discov. 8, 196-215 (2018).
38. Aref, A. R. et al. 3D microfluidic: Ex vivo culture of organotypic tumor spheroids to model immune checkpoint blockade. Lab Chip 18, 3129-3143 (2018).
39. Wan, C. et al. Enhanced Efficacy of Simultaneous PD-1 and PD-L1 Immune Checkpoint Blockade in High-Grade Serous Ovarian Cancer. Cancer Res. 81, 158-173 (2021).
40. Odunsi, A. et al. Fidelity of human ovarian cancer patient-derived xenografts in a partially humanized mouse model for preclinical testing of immunotherapies. J. Immunother. Cancer 8, 1-13 (2020).

### Example 3

### Engineering Chimeric Antibody Signaling and Secreting (CASS) B Cells to Achieve Cancer Cures

Cancer cells are a life form that has learned to commandeer our immune system for its own growth advantage. They do this through impairment of our cellular DNA repair mechanisms that lead to upregulation of growth factors and their receptors that results in uncontrolled tumor growth. This molecular highjacking also leads to surface expression and secretion of molecules involved in immune checkpoint blockade (ICB). Immunotherapy though anti-PD1/PDL1 blockade represents an important advance in the cancer field, and is a front-line or standard therapeutic option for various cancers, including non-small-cell lung cancer, melanoma, colorectal cancer, and renal cell carcinoma (1-3). However, while treatment successes are well documented, they most often do not result in cancer cures. We must do better.

Cellular therapies are a way to harness the immune system to kill cancer cells. For example, T cell receptor (TCR) and chimeric antigen receptor (CAR) T cells which are able to home to and target the cancer cells.

Further, embodiments herein comprise a new form of cellular therapy called Chimeric Antibody Signaling and Secreting (CASS) B cells that will utilize B cells of the humoral immune system to do what they do best-secrete high levels of antibodies. These CASS B cells will be engineered to recognize tumor associated antigens (TAA) through an engineered B cell receptor (BCR), which upon TAA engagement will activate the CASS B cell and induce the production of high levels of an immunomodulatory bispecific antibody (BsAb) at the tumor site. This will allow for reversal of the immunosupressive tumor microenvironment as it does when immune checkpoint blockade monoclonal antibodies (mAbs) are delivered systemically. BsAb secretion can be conditionally expressed only upon CASS B cell engagement with TAAs, and largely localized to the tumor site. Without wishing to be bound by theory, this is a step toward a new cellular therapy where locally secreted monoclonal antibodies are devoted to changing the tumor microenvironment and restoring local anti-tumor immunity.

Monoclonal antibody (mAb) drugs that directly kill cancer cells, act as immune checkpoint blockade inhibitors or disrupt tumor vasculature are among the most promising anti-cancer therapeutics under development. However, the idea of engineering human B cells to seek out cancer cells and secrete these mAbs at the tumor site in vivo is new and can provide a powerful new way to treat both primary and metastatic tumors. It can also provide a lifelong anti-tumor immune surveillance system to prevent cancer reoccurrence and achieve “CURES”. A role of B cells in our immune system is to recognize foreign invaders and eliminate them by production of antibodies that bind and clear the threat whether a microbe or cancer cell. They accomplish this by expressing a membrane bound antibody that acts as a B cell Receptor (BCRs) that bind the tumor antigen, causing the B cell to switch from a BCR expressing cell to an antibody secreting cell. After BCR engagement there is rapid tyrosine phosphorylation mediated by two primary tyrosine kinases, Lyn and Syn, and calcium ion polarization. These biochemical events lead to activation of downstream signaling pathways, resulting in further downstream activation of NF-kB and NFAT signaling and ultimately in B cell expansion and robust mAb secretion (5). We will engineer Chimeric Antibody Signaling and Secreting (CASS) B cells that will take advantage of these signaling pathways to induce clonal CASS B cell expansion and secretion of an immunomodulatory BsAb at the tumor site.

Without wishing to be bound by theory, the development of the CASS B cell platform can benefit a multitude of clinical indications that are sensitive to checkpoint blockade inhibitors or have an immunosuppressive microenvironment. Anti-PD1/PDL1 therapy has had a transformative effect on the treatment of NSCLC, for example, becoming a frontline therapy for many patients. However, there are limitations to the efficacy, durability, and scope of use for this therapy. (6-7). We will use NSCLC as an experimental model utilizing anti-PD1/anti-TIGIT bispecific antibodies under development in our lab.

To target the CASS B cell to the NSCLC tumor site, an engineered, membrane bound, single chain IgG BCR against mesothelin (MSLN) will be used. The plasmid will also contain a second cassette driven by a NFAT or NF-kB responsive element that drives secretion of an anti-TIGIT/anti-PD1 BsAb. Upon localization to the tumor site and BCR activation, BsAb expression will be induced by the native signaling pathway upon BCR engagement of MSLN on NSCLC. Since this will be an inducible expression system, there will be a localized area of high antibody concentration at the tumor site, significantly decreasing on-target, off-tumor effects.

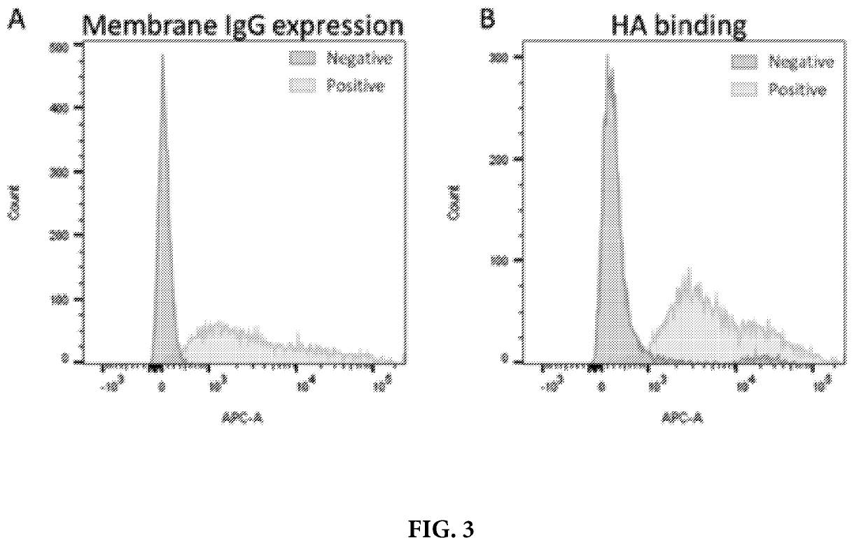

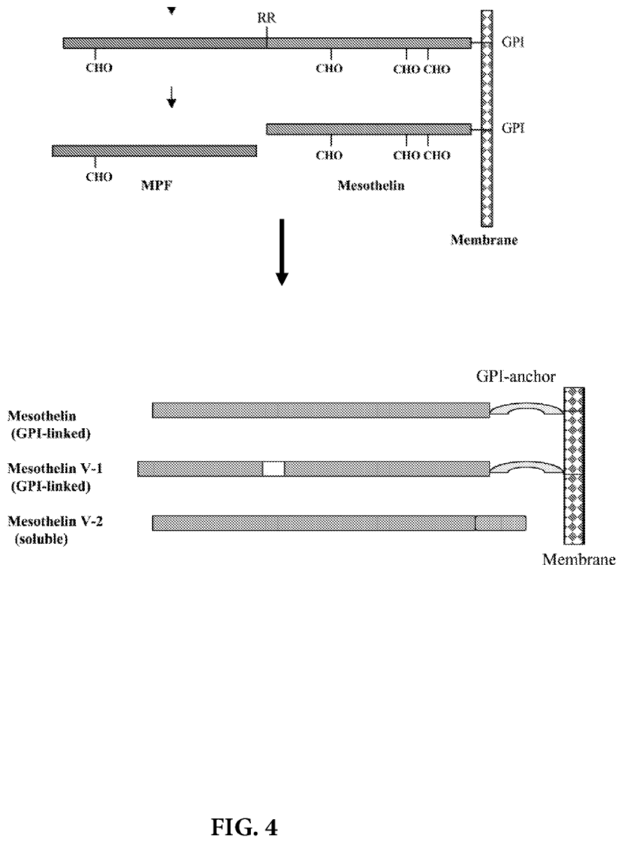

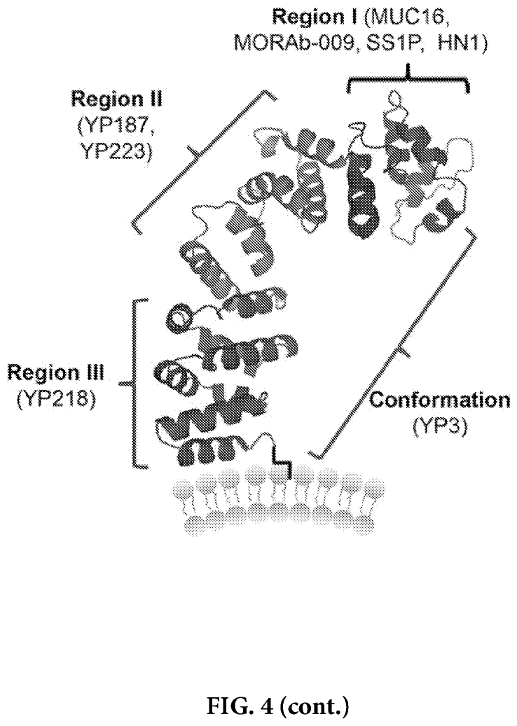

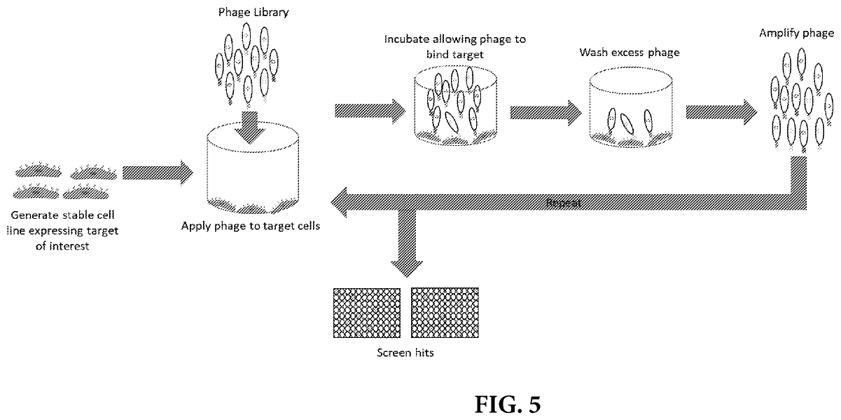

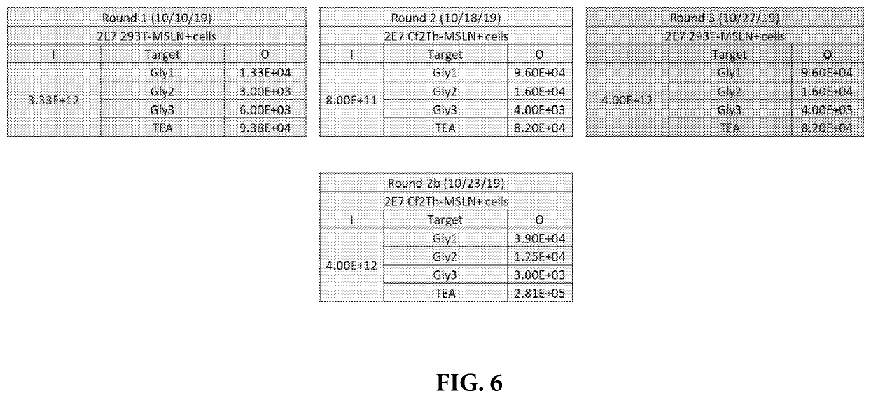

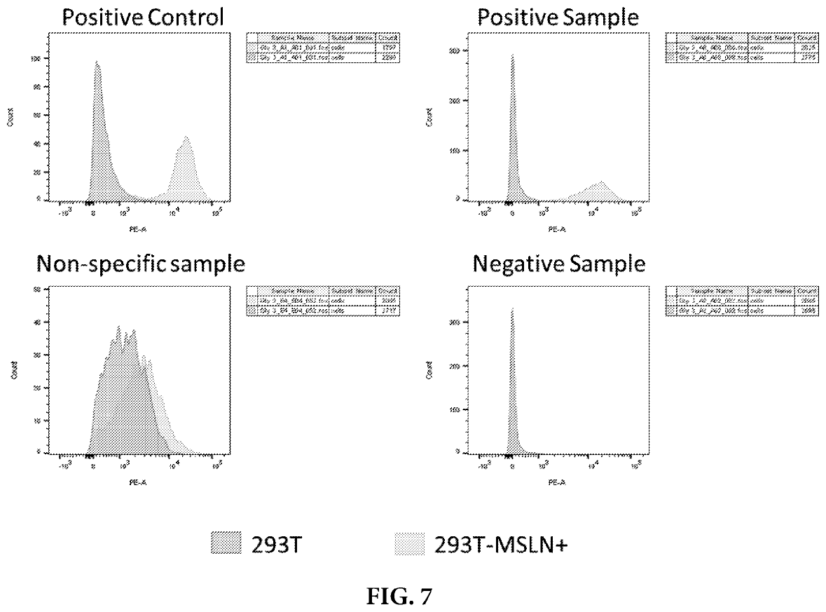

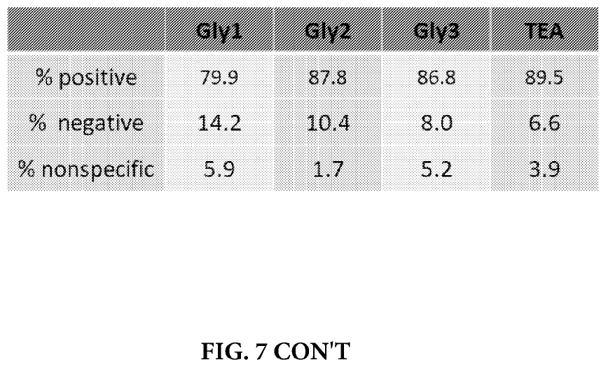

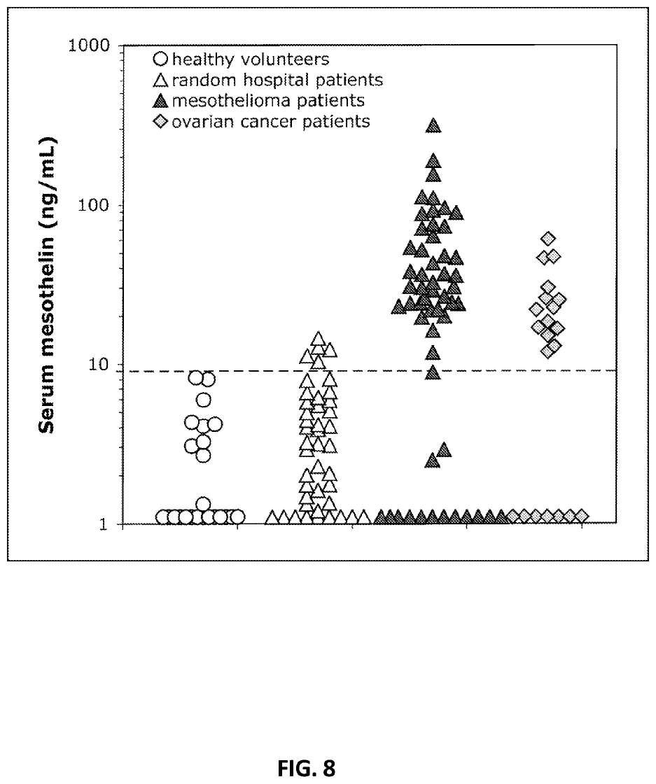

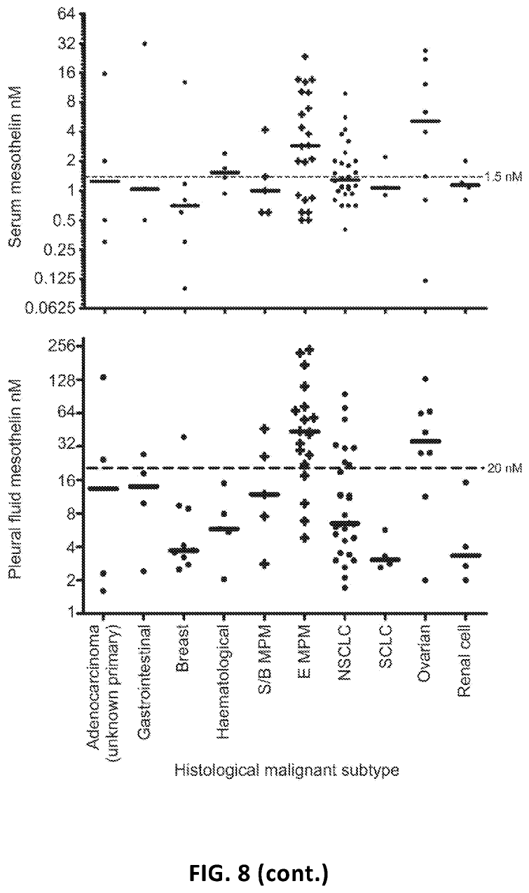

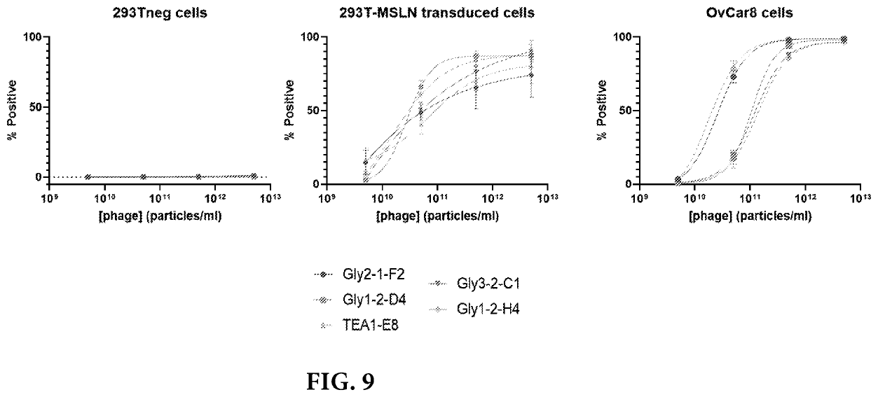

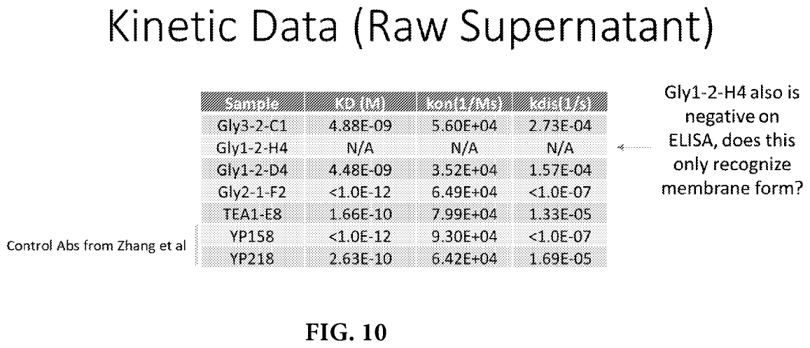

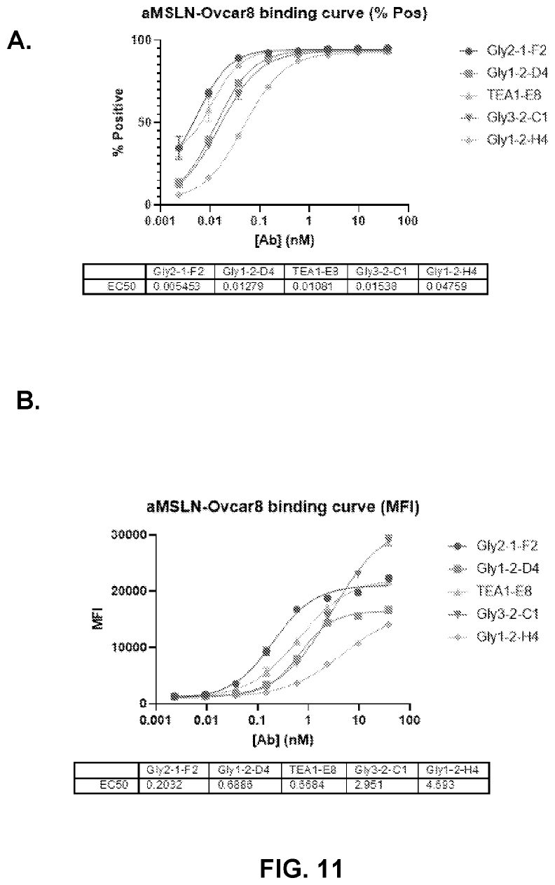

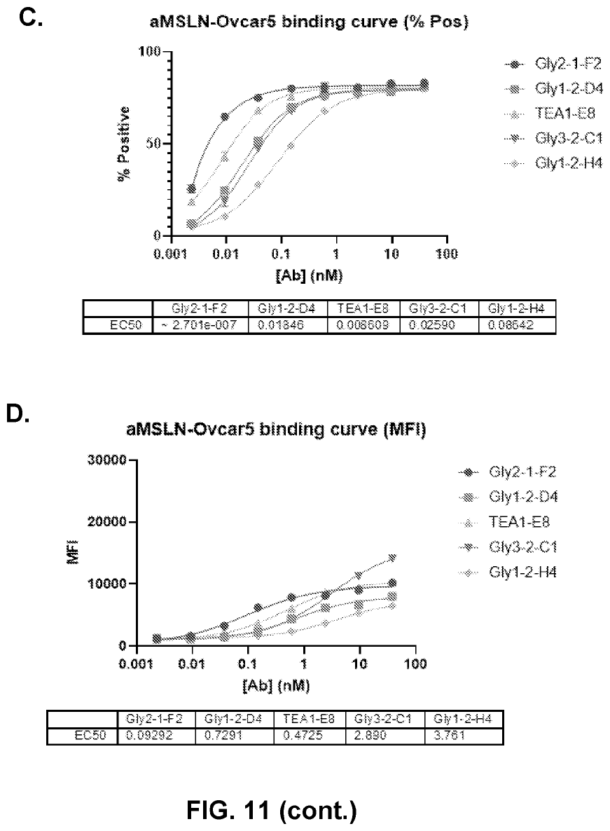

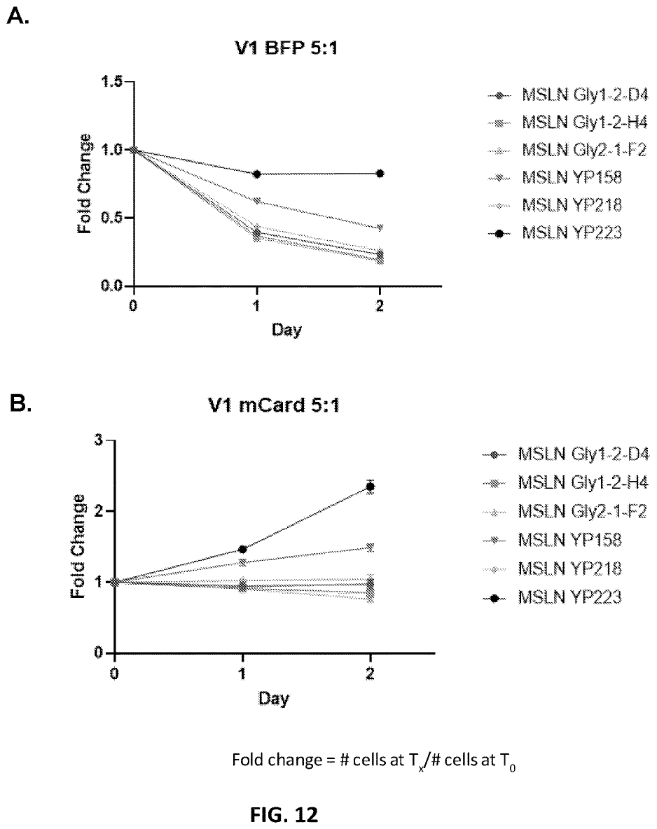

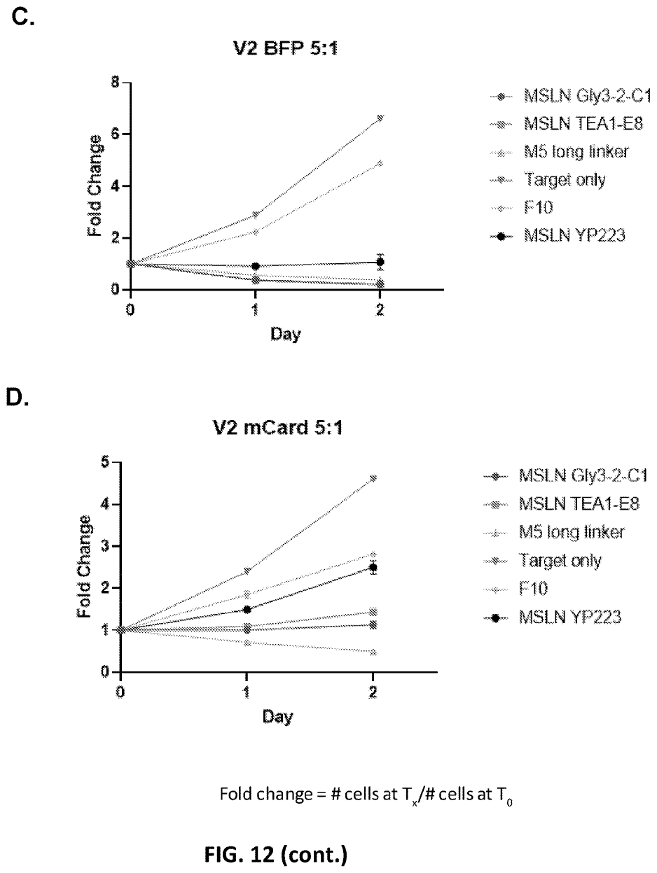

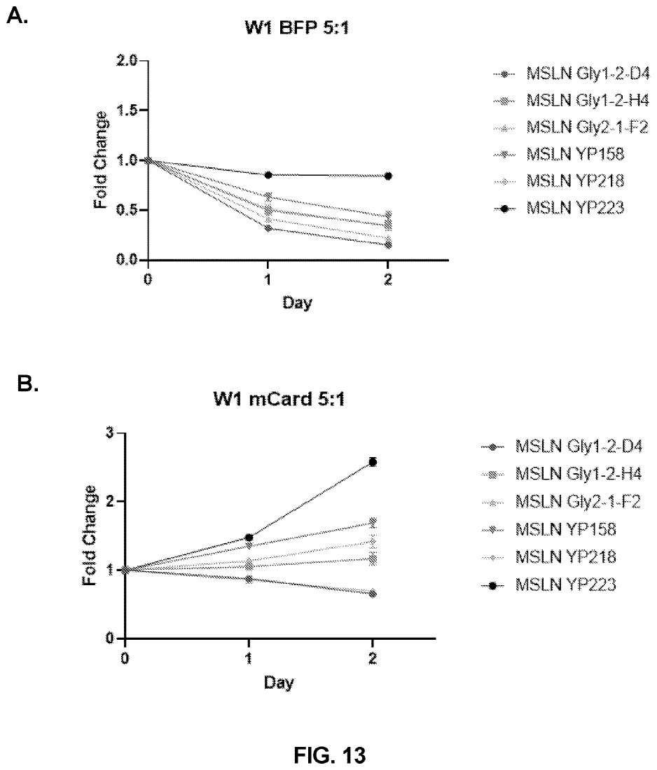

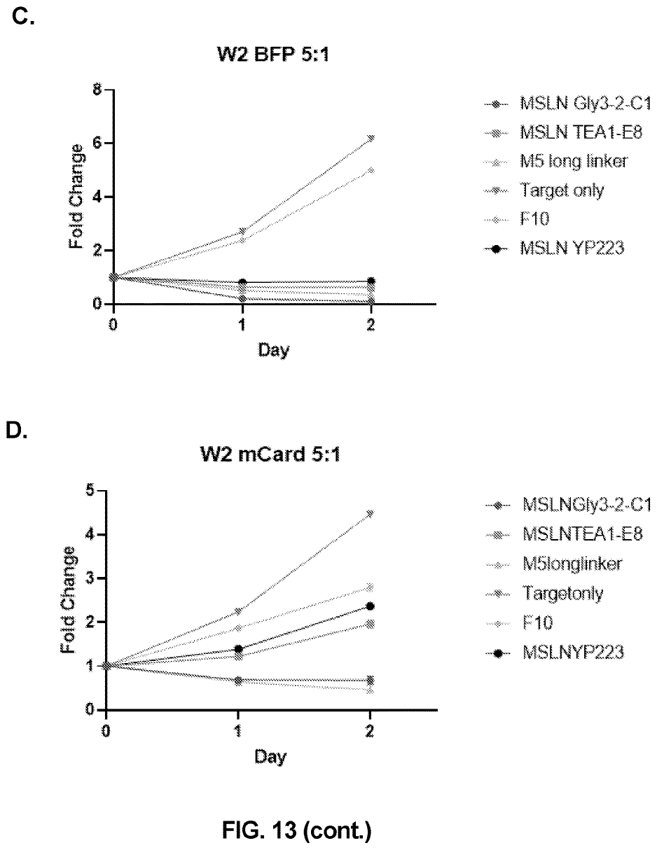

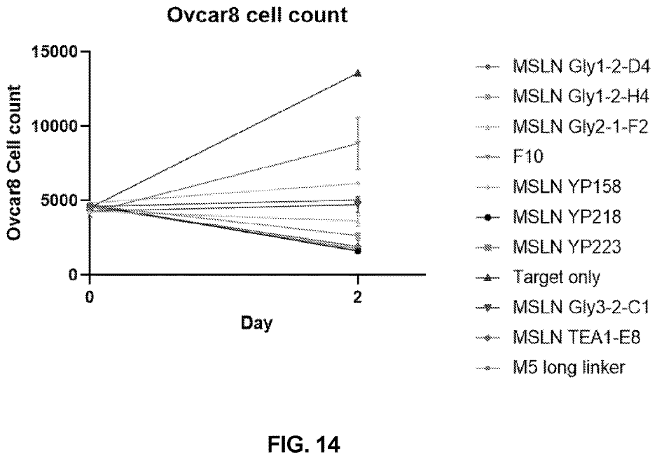

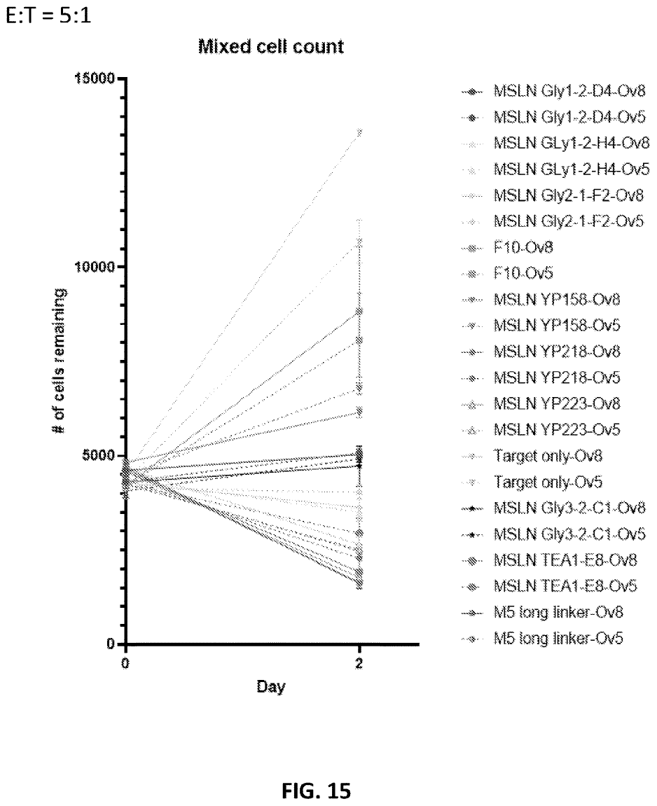

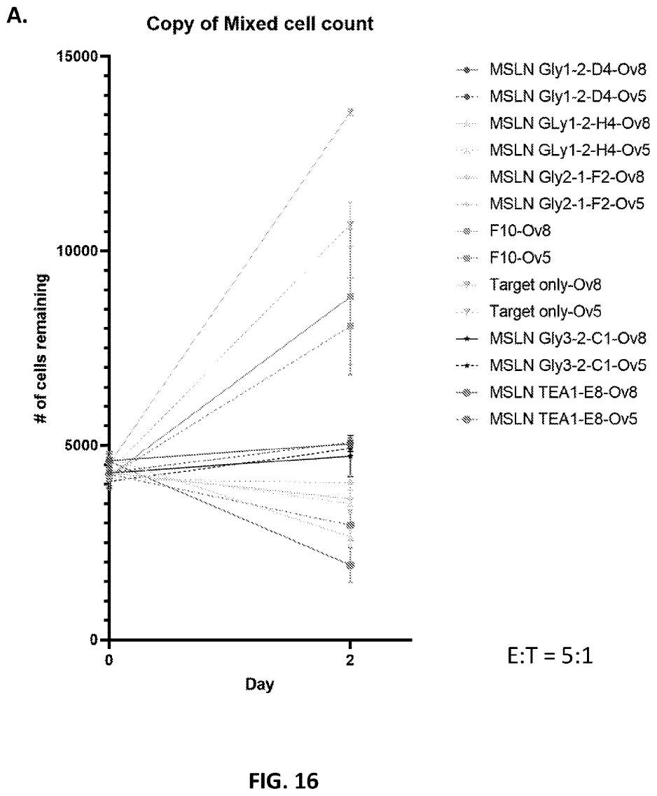

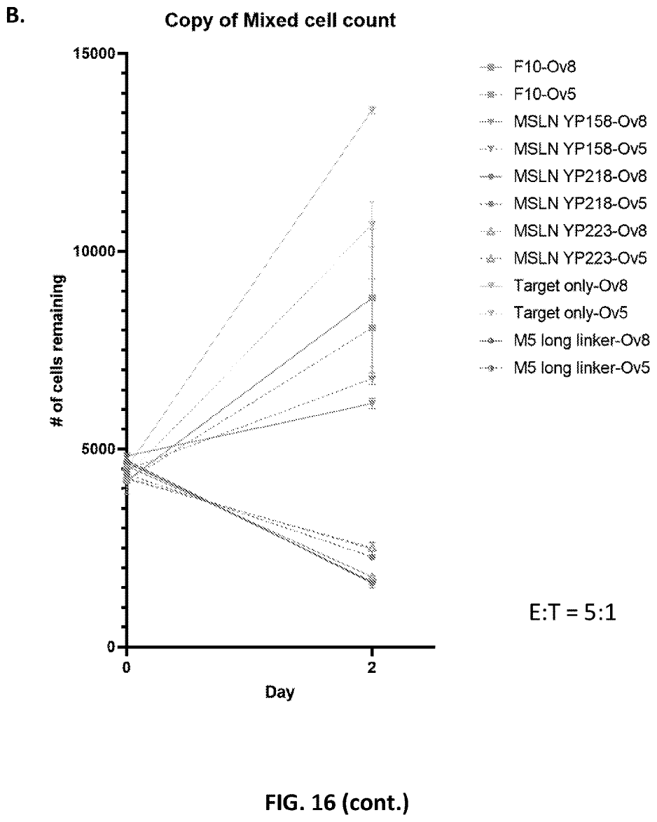

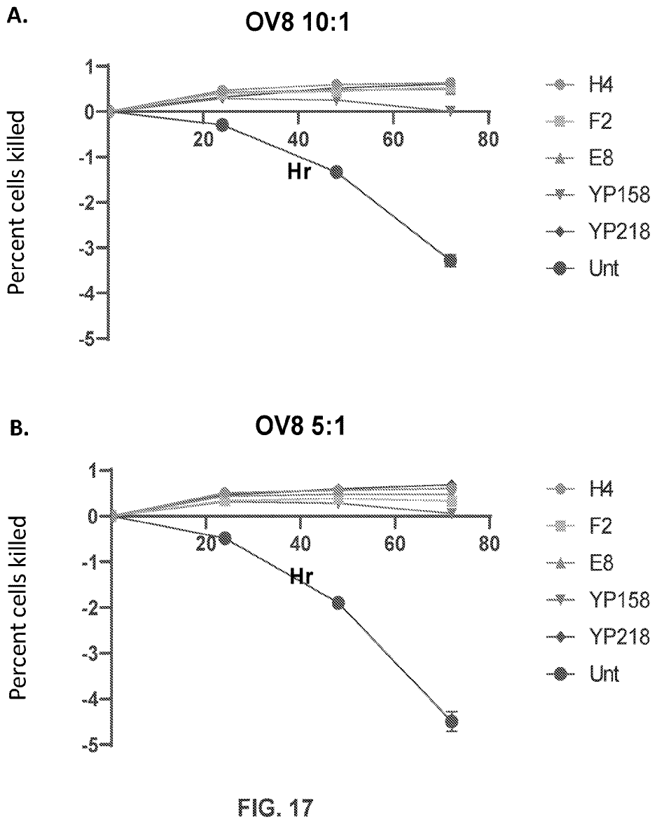

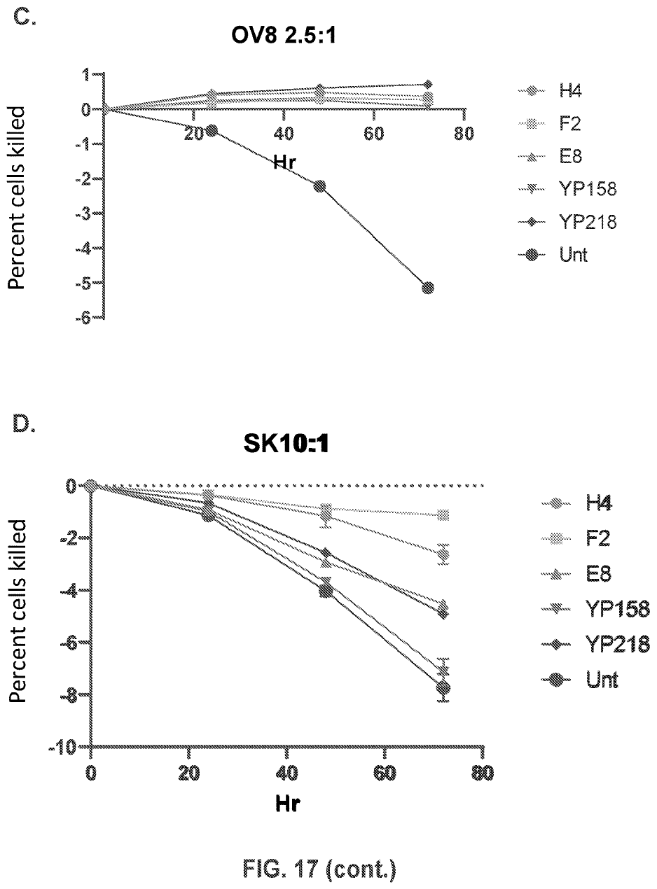

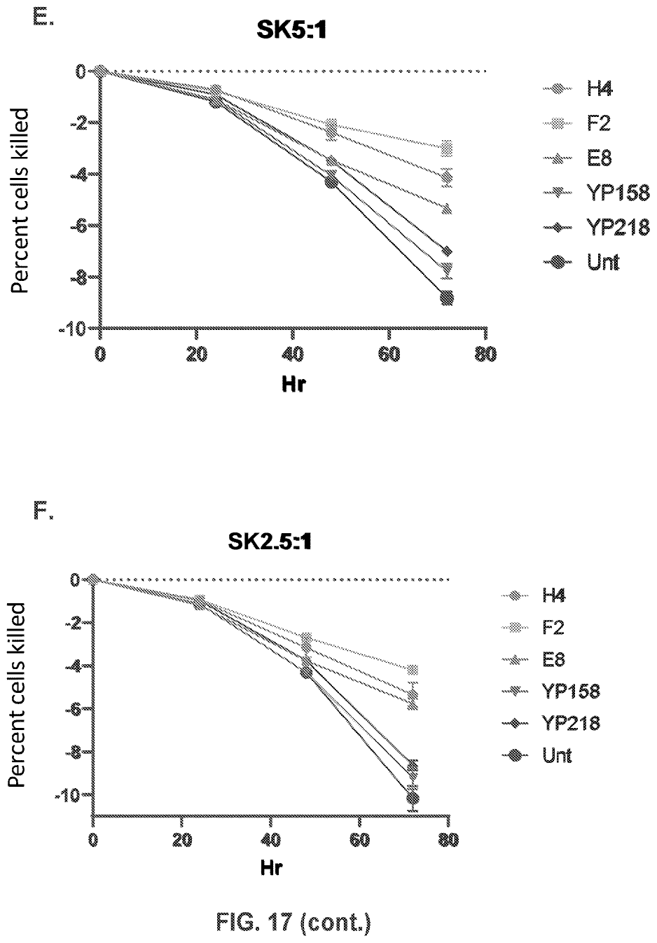

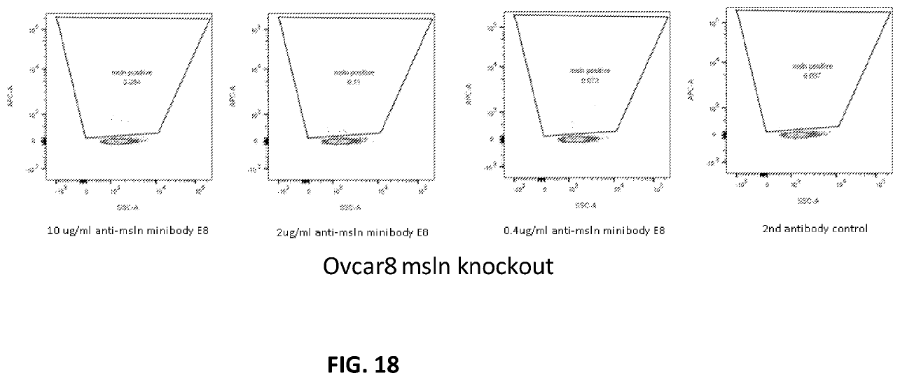

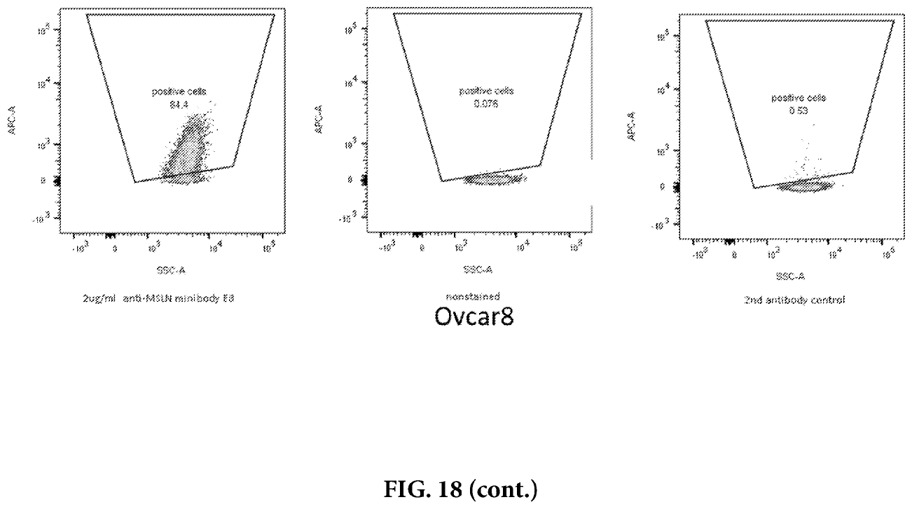

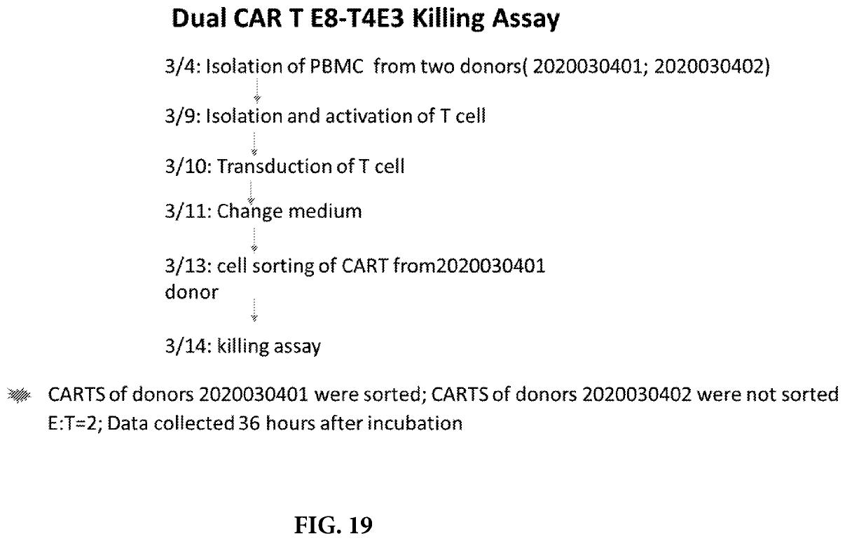

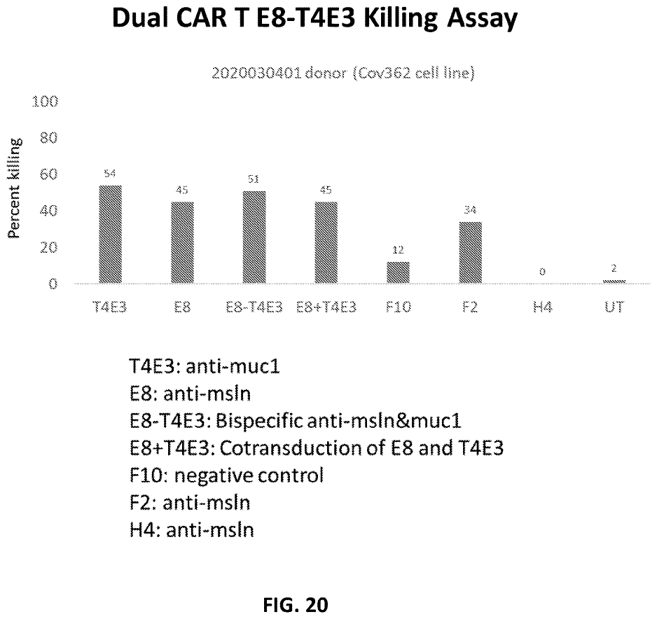

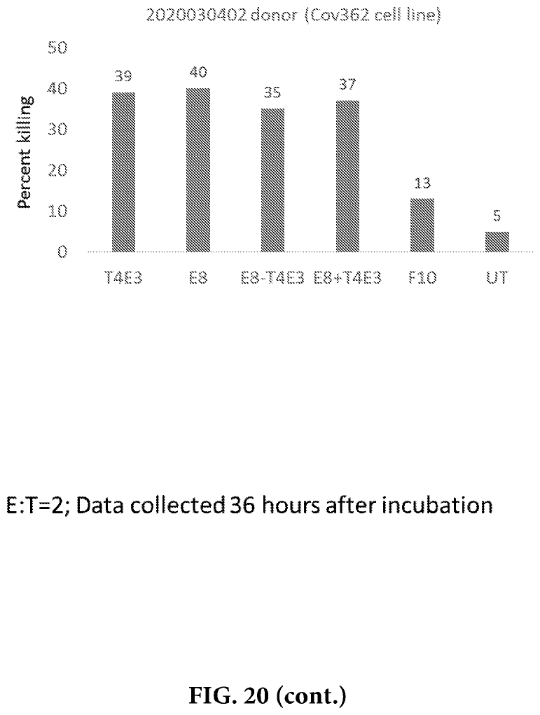

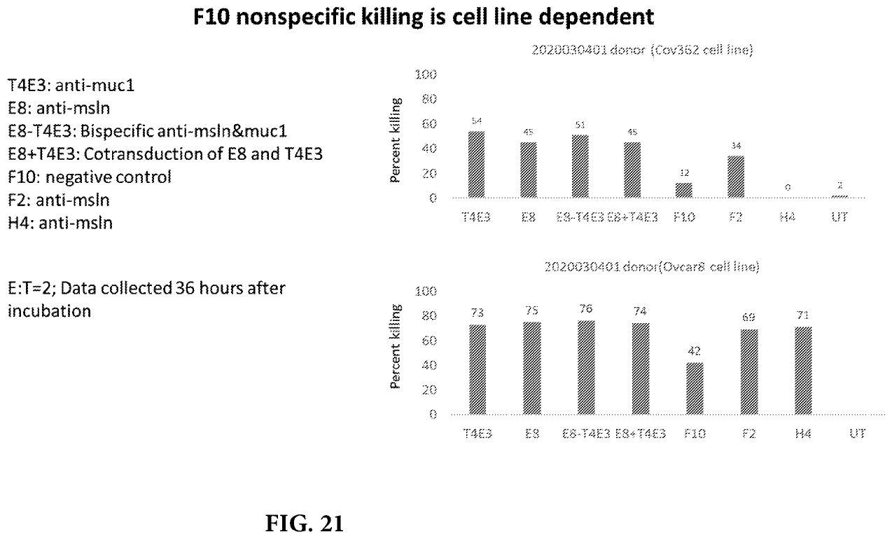

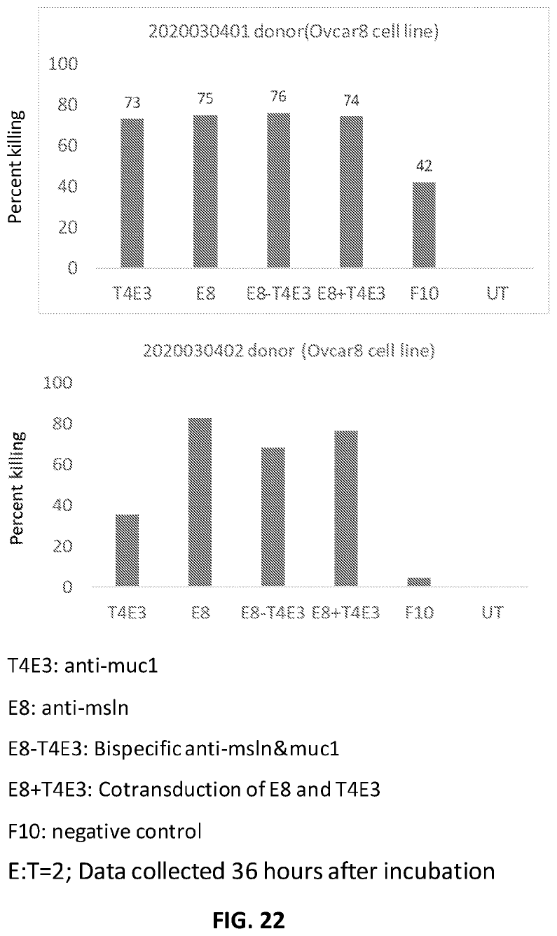

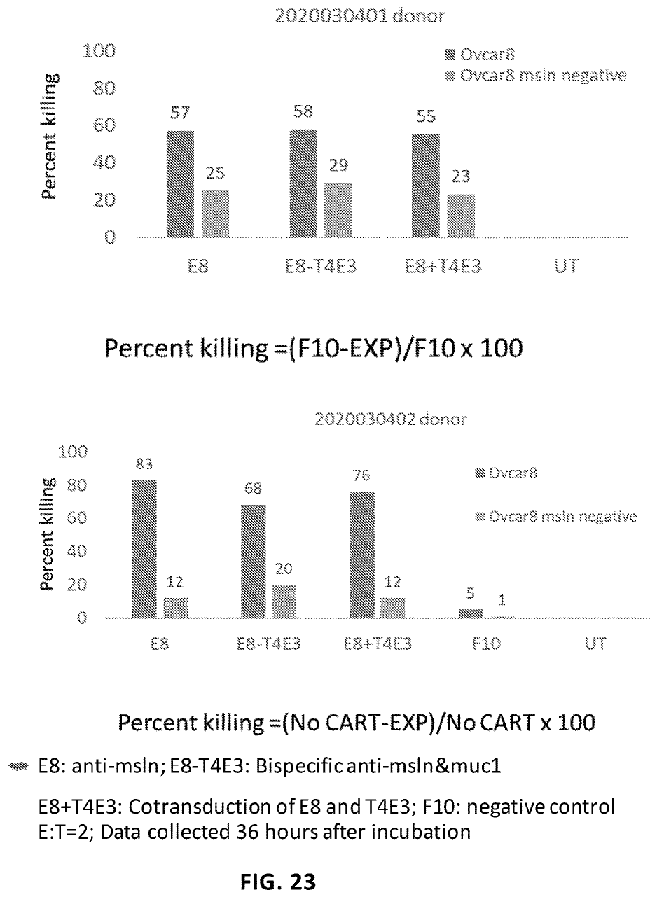

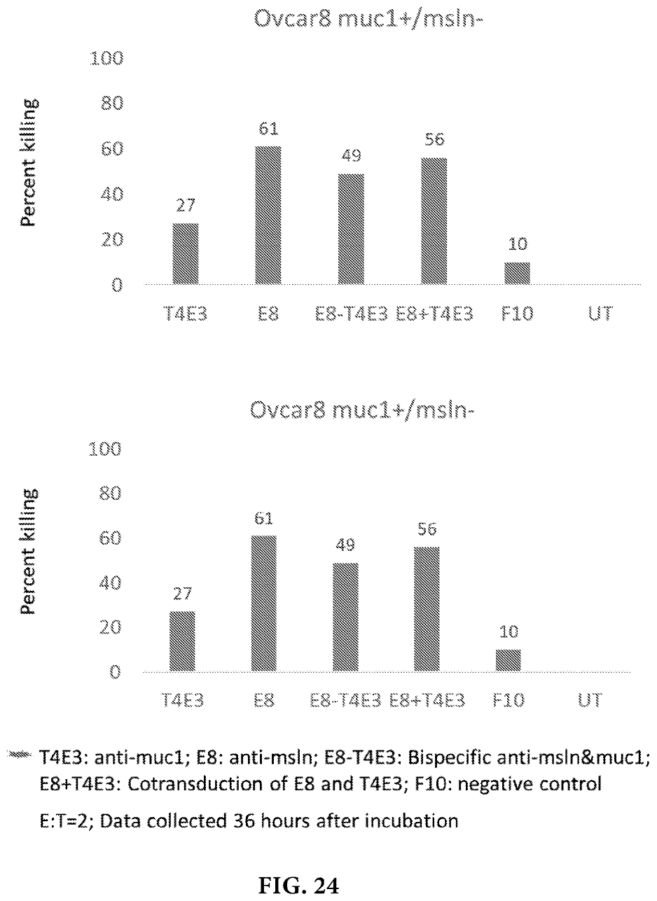

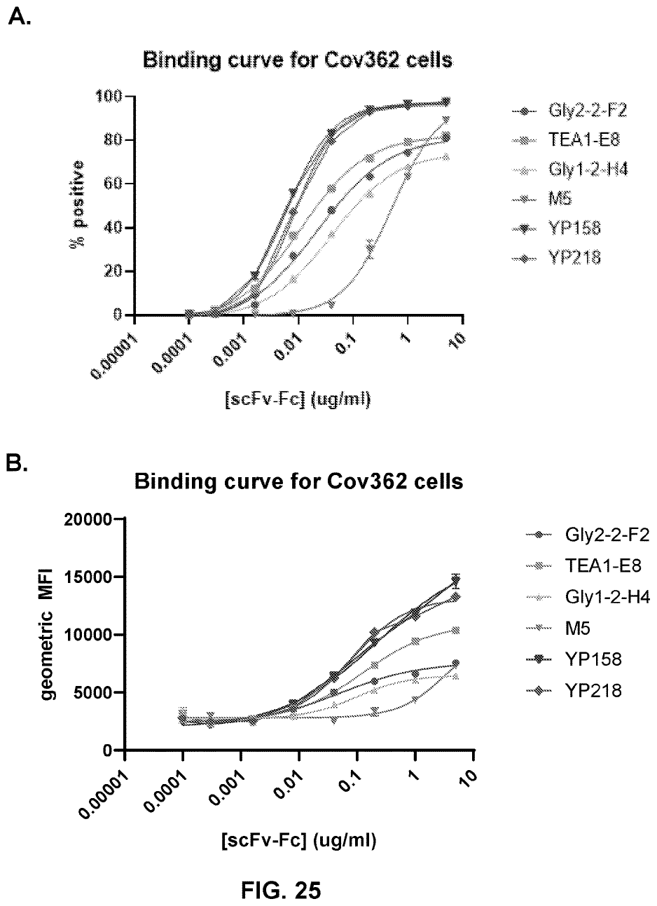

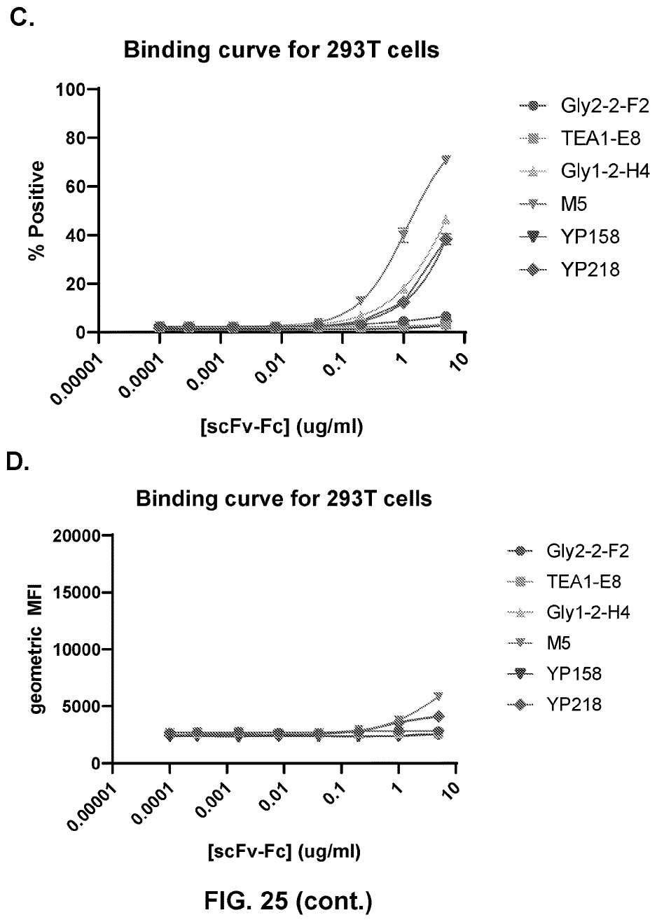

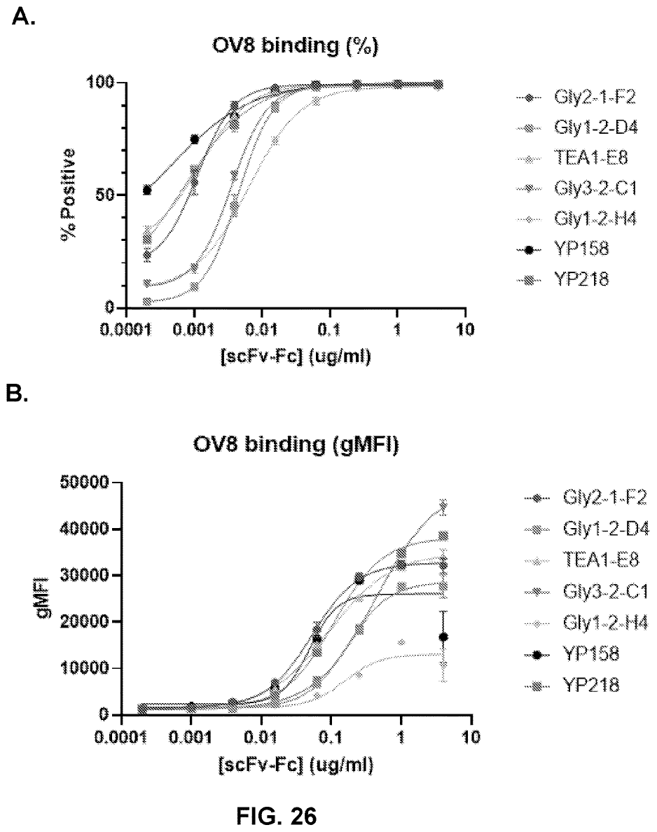

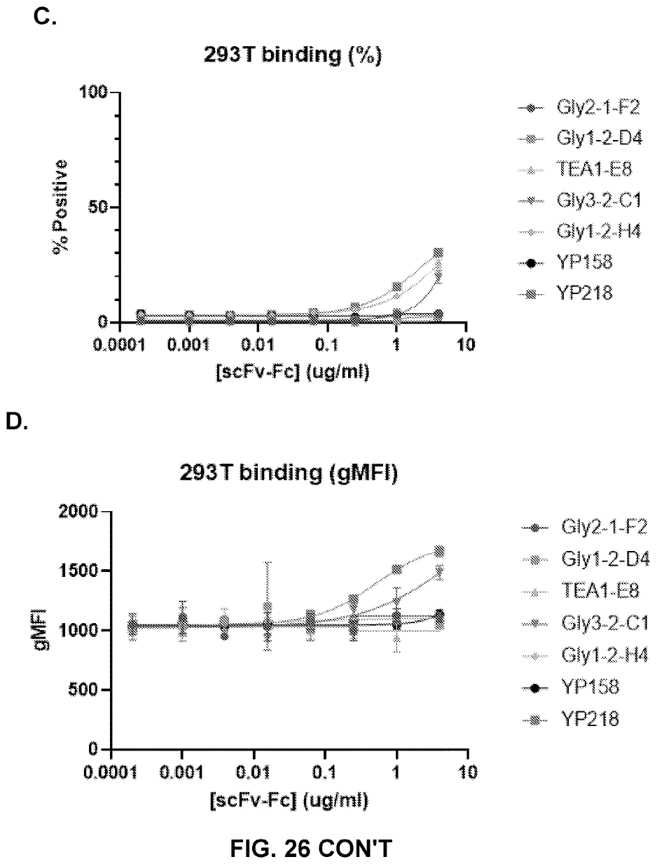

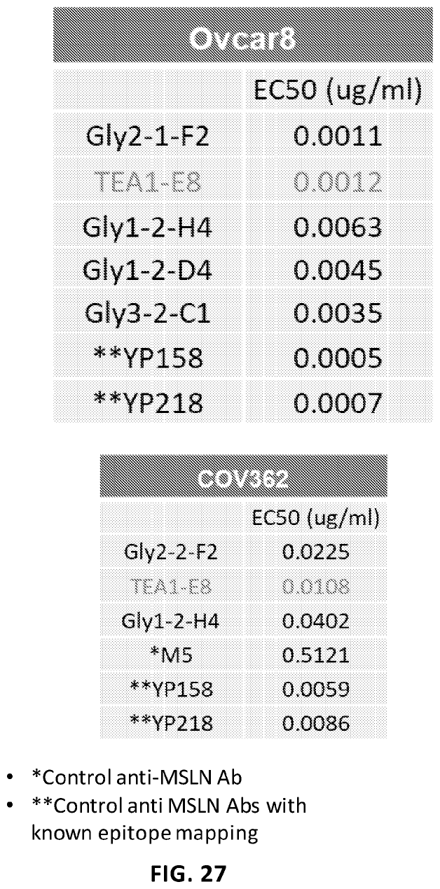

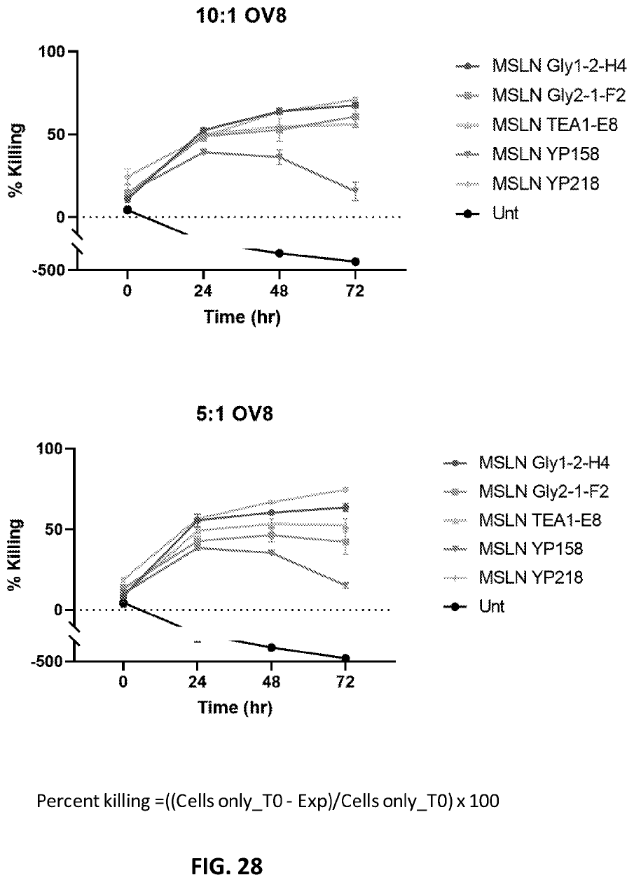

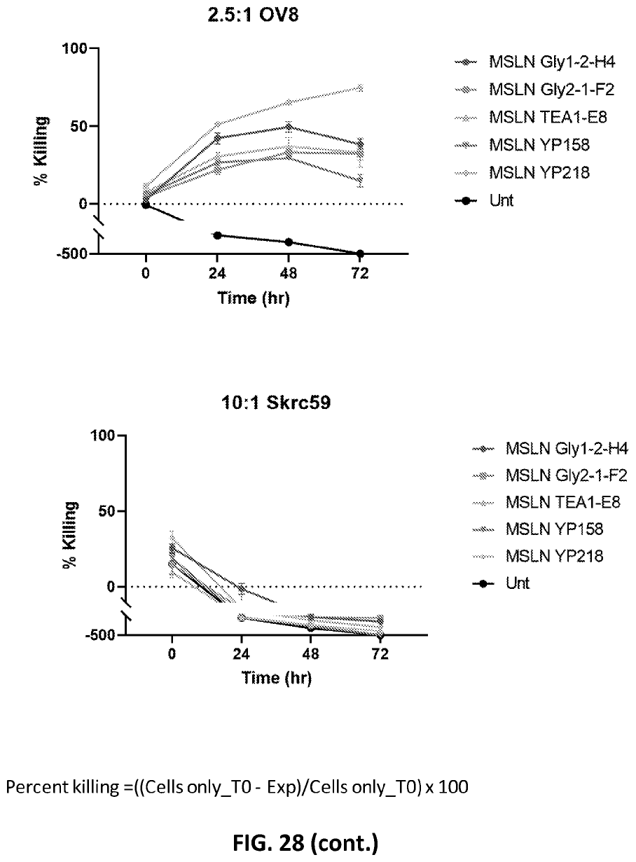

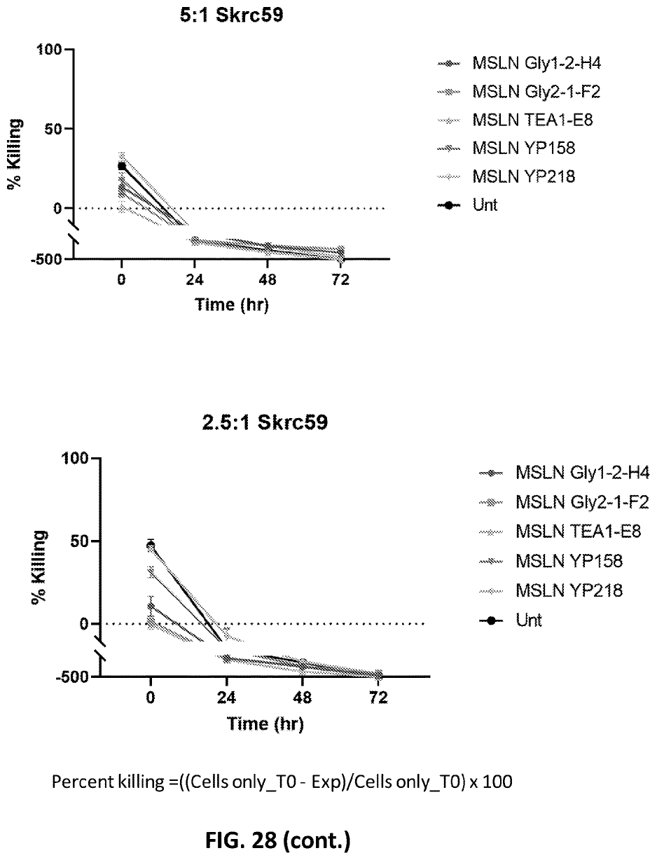

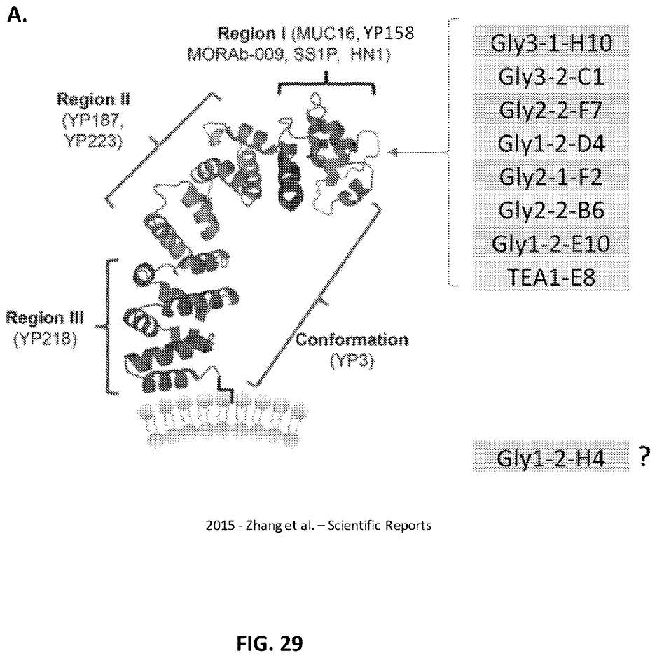

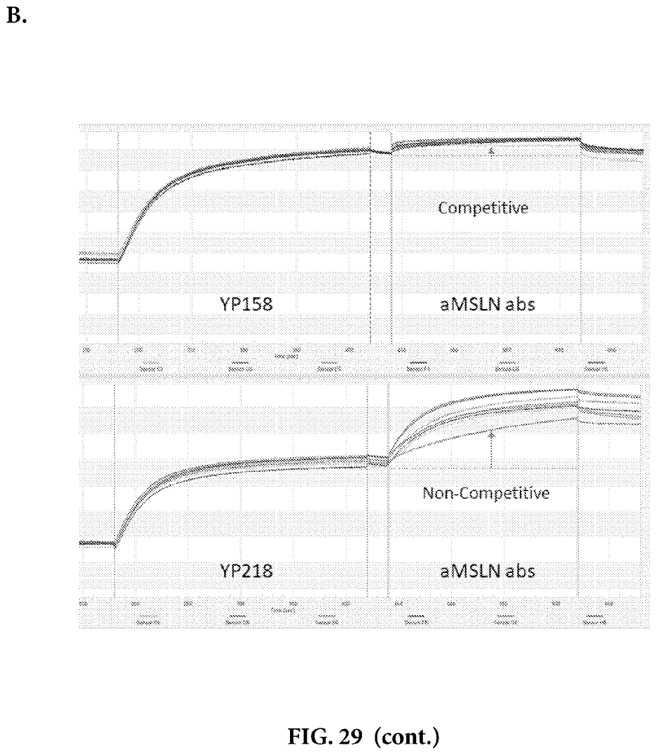

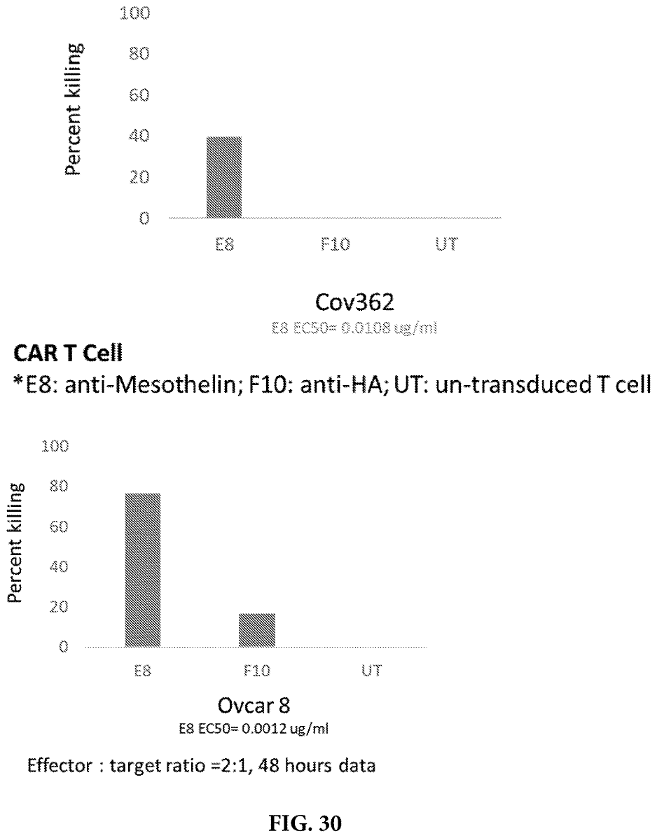

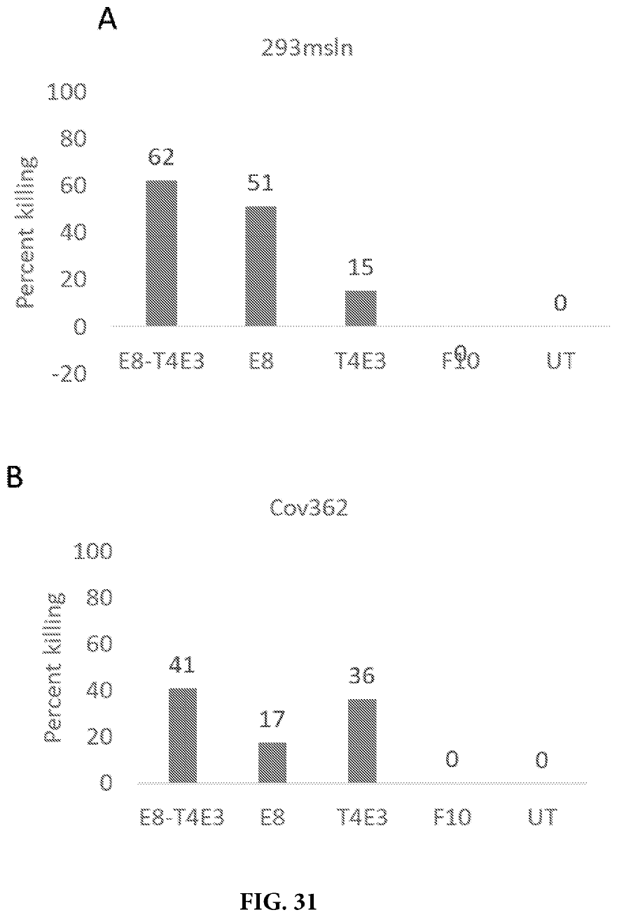

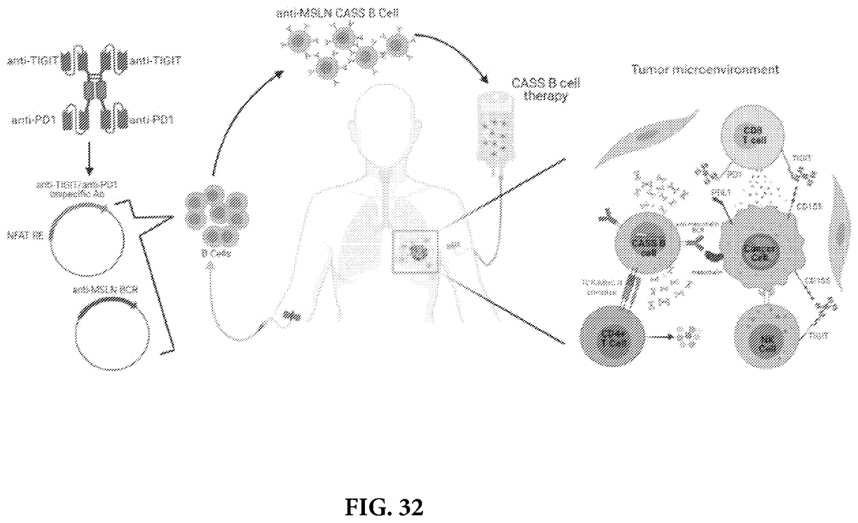

The CASS B cell will be designed in two parts. We will successfully transfer the engineered BCR into the B cell. To efficiently transduce B cells, lentivirus particles will be pseudotyped with Gibbon-ape leukemia virus (GALV) or engineered baboon envelope glycoproteins and in the transfer vector BCR expression will be driven from a spleen focus-forming virus (SFFV) or human elongation factor-1 alpha (EF1α) promotor (8). Next, we will validate a NFAT and/or NF-kB inducible expression cassette for BsAb secretion (9). To decide which response element to use, plasmids will initially be designed to express GFP. Raji cells and primary B cells will be transduced with the engineered anti-MSLN BCR and NFAT/NF-kB inducible GFP lentiviral vector, and soluble, biotinylated MSLN will be added to the culture media with streptavidin to cross-link BCRs. GFP expression will then be measured to determine the optimal response element. We will construct an engineered membrane bound IgG (memIgG) using an anti-influenza antibody. FIG. 3 shows that our memIgG construct expresses at high levels and is functionally active as it is able to bind soluble HA.

Next, CASS B cells will be constructed by lenvtivirus transduction with the vector encoding the tumor specific anti-MSLN BCR and an inducible BsAb replacing GFP. CASS B cells secretion of anti-TIGIT/PD1 BsAb will be quantified, first using soluble biotinylated MSLN+streptavidin to induce expression, followed by co-incubation with MSLN+ A549 NSCLC cells. Secondary experiments will test for in vitro inhibition of exhaustion by co-culturing CASS B cells with CD3+ T cells and A549 cells expressing various combinations of PDL1 and CD155 (ligand of TIGIT). In addition to measuring soluble antibody concentration, cellular activation/exhaustion will be measured by FACS staining. These tumor cells will also be stained for binding of CASS B cell shed anti-MSLN. In vivo experiments will next be carried out in humanized PBL NSG-SGM3 mice bearing A549 tumors to validate CASS B cell efficacy and persistence. Efficacy will be measured by local BsAb secretion by immunohistochemical staining, increased B cell population around the tumor site, and change in tumor size. We will also measure BsAb leakage into periphery by examining serum secreted BsAb concentration. To measure persistence, CASS B cells will be detected in both peripheral blood and the tumor microenvironment (TME). Finally, scRNAseq will be used to analyze the effect of the BsAb in modulating the TME. Compared to systemic delivery, there will be a localized area of high BsAb concentration centered around the tumor, leading to improved outcome and tumor elimination.

The goal of developing new cancer therapies can be aimed at achieving “CURES”. Restoring host anti-tumor immunity at the tumor site is achievable by understanding the immune elements that are common or “public” to individuals. Once this is done, we must develop an internal immunosurveillance system that will forever be present to prevent cancer from reoccurring. We will develop a new immune surveillance system using B cells of the immune system that have never been developed for this purpose.

We will validate the feasibility of engineering Chimeric Antibody Signaling and Secreting (CASS) B cells to target mesothelin (MSLN) expressing NSCLC cells. These anti-MSLN CASS B cells will home to the tumor site, where they will secrete high levels of a bispecific anti-TIGIT/anti-PDL1 antibody that will act as a dual checkpoint blockade inhibitor. This will result in a dynamic shift in the tumor microenvironment that will help reverse T cell exhaustion and restore anti-tumor immunity. An important and practical point is that this combination cellular immunotherapy will be a one-time administration for the lifetime of the patient.

### REFERENCES CITED IN THIS EXAMPLE

1. Rolfo C, Caglevic C, Santarpia M, Araujo A, Giovannetti E, Gallardo CD, Pauwels P, Mahave M. Immunotherapy in NSCLC: A Promising and Revolutionary Weapon. Adv Exp Med Biol. 2017; 995:97-125. doi: 10.1007/978-3-319-53156-4_5.PMID: 28321814 Review.
2. M.-O. Grimm, K. Leucht, V. Grünwald, S. Foller, New First Line Treatment Options of Clear Cell Renal Cell Cancer Patients with PD-1 or PD-L1 Immune-Checkpoint Inhibitor-Based Combination Therapies. J. Clin. Med. 9, 565 (2020).
3. X. Wu, et al., Application of PD-1 Blockade in Cancer Immunotherapy. Comput. Struct. Biotechnol. J. 17, 661-674 (2019).
4. Ryeong Lee B, Sehyun C, Moon J, Joon Kim M, Lee H, Wan Ko H, Chul Cho B, Sup Shim H, Hwang D, Ryun Kim H, and Ha S-J. Combination of PD-L1 and PVR determines sensitivity to blockade. PD-1 JClinsite 2020; 5 (14): e128633. https://doi.org/10.1172/jci. insight.128633.
5. Tolar P, Hanna J, Krueger P D, Pierce S K. The constant region of the membrane immunoglobulin mediates B cell-receptor clustering and signaling in response to membrane antigens. Immunity. 2009; 30 (1): 44-55. Epub 2009 Jan. 13. doi: 10.1016/j.immuni.2008.11.007. PubMed PMID: 19135393; PMCID: PMC2656684.45.
6. Bendell J C, Bedard P, Bang Y J, et al: Phase Ia/Ib dose-escalation study of the anti-TIGIT antibody tiragolumab as a single agent and in combination with atezolizumab in patients with advanced solid tumors. 2020 AACR Virtual Annual Meeting II. Abstract CT302.
7. M.-J. Ahn, et al., 1400P Vibostolimab, an anti-TIGIT antibody, as monotherapy and in combination with pembrolizumab in anti-PD-1/PD-L1-refractory NSCLC. Ann. Oncol. 31, S887 (2020).
8. Levy C, Fusil F, Amirache F, Costa C, Girard-Gagnepain A, Negre D, Bernadin O, Garaulet G, Rodriguez A, Nair N, Vandendriessche T, Chuah M, Cosset F L, Verhoeyen E. Baboon envelope pseudotyped lentiviral vectors efficiently transduce human B cells and allow active factor IX B cell secretion in vivo in NOD/SCIDgammac(−/−) mice. J Thromb Haemost. 2016; 14 (12): 2478-92. Epub 2016 Sep. 30. doi: 10.1111/jth. 13520. PubMed PMID: 27685947.
9. Uchibori R, Teruya T, Ido H, Ohmine K, Sehara Y, Urabe M, Mizukami H, Mineno J, Ozawa K. Functional Analysis of an Inducible Promoter Driven by Activation Signals from a Chimeric Antigen Receptor. Mol Ther Oncolytics. 2019; 12:16-25. Epub 2019 Jan. 22. doi: 10.1016/j.omto.2018.11.003. PubMed PMID: 30662937; PMCID: PMC6325072.

### Example 4

### Engineering Chimeric Antibody Signaling and Secreting (CASS) B Cells to Achieve Cancer Cures

Immune checkpoint blockade inhibitors (CBI) and CAR T cells have revolutionized the way we treat cancer. While both of these therapies engage the patient's immune system, neither is able to proactively initiate an anti-tumor immune response and significant limitations exist in their scope of use and efficacy. To address this, we can develop Chimeric Antibody Secreting and Signaling (CASS) B cells, which express an engineered, tumor targeting B cell receptor and upon engagement, will secrete high levels of a dual-targeted bispecific CBI antibody locally at the tumor site. As B cells also serve as professional antigen presenting cells, they can process and present antigens on MHC class II molecules, further enhancing immune cell recognition of the tumor and assisting in neoantigen spreading. A key component of immunologic memory, CASS B cells will simultaneously recruit a wide range of immune cells and reverse tumor infiltrating lymphocyte exhaustion, providing a surveillance program protecting against tumor metastasis and recurrence.

Non-small cell lung cancer (NSCLC) was chosen as a model to develop an anti-MSLN directed CASS B cell secreting an immunomodulatory anti-PD1/TIGIT bispecific antibody (bsAb). Aim 1 will focus on the development of the CASS B cell platform and at the conclusion of this stage, we will have identified the three antibodies and optimized the signaling domains that comprise CASS B cells. In Aim 2, in vitro characterization and efficacy testing will be performed, resulting in a clear understanding of the linkage between CASS B cell activation and bsAb secretion, while providing critical analysis of CASS B cell efficacy in comparison to CAR T cells at both a functional and molecular level. Aim 3 will execute in vivo experiments using cell line derived and patient derived NSCLC models in humanized mice. Multiparameter flow cytometry, single cell RNA sequencing, and immunohistochemistry will provide a detailed assessment of the molecular and mechanistic efficacy of the immunomodulatory bsAb and the CASS B cell platform.

We can develop a new type of combination cellular immunotherapy, Chimeric Antibody Secreting and Signaling (CASS) B cells. These B cells will express an engineered tumor associated antigen (TAA) targeting B cell receptor (BCR), and upon engagement, will secrete high levels of a checkpoint blockade inhibitor (CBI) bispecific antibody (bsAb) locally at the tumor site. This will allow for reversal of the immunosuppressive tumor microenvironment (TME) and restoration of the patient's natural innate and adaptive immunity to eliminate cancerous cells. In addition, unlike T cells, B cells act as professional antigen presenting (APC) cells and can enhance tumor cell recognition and assist in neoantigen spreading, thereby leading to both reversal of tumor infiltrating lymphocyte (TIL) exhaustion and induction of a broader and more robust anti-tumor immune response. We will engineer an anti-PD1/TIGIT bsAb secreting CASS B cell that targets mesothelin (MSLN) for the treatment of NSCLC¹⁻³.

The first objective comprises the construction and optimization of the engineered anti-MSLN IgG-BCR, the anti-PD1/TIGIT bsAb to be delivered at the tumor site, and the inducible response element (RE) that drives bsAb expression. Panels of anti-MSLN, anti-PD1, and anti-TIGIT antibodies were identified by our lab and functional assays will identify lead candidates. Concurrent efforts will focus on the development of the inducible response elements and optimization of B cell transduction conditions.

The second objective is the functional validation of the anti-MSLN CASS B cell in vitro. Activation assays will be used to quantify bsAb and cytokine secretion levels. Patient-derived organotypic tumor spheroids (PDOTS) will be used to validate CASS B cell efficacy and for molecular/mechanistic comparisons with CAR T cells via cytokine profiling, IHC, and single cell RNA sequencing (scRNAseq).

Another objective will be to utilize HLA matched, humanized mice to generate cell line derived (CDX) and patient derived xenograft (PDX) models of NSCLC to test CASS B cell efficacy. Models will be paired with various analytical techniques (IHC, flow cytometry, scRNAseq) to further interrogate the effects of CASS B cell therapy on the surrounding TME.

Background: Checkpoint blockade inhibitor (CBI) monoclonal antibodies (mAbs) and adoptive cellular therapies have had a transformative effect on cancer therapies, changing the focus from killing tumor cells to activating a patients natural anti-tumor immunity and reversing the immunosuppressive tumor microenvironment (TME). While these represent some of the most promising anti-cancer therapeutics to date, only a small subset of patients experiences complete or durable responses and many experience immune related adverse events (irAE) of varying severity⁴⁻⁷. To counter this, combination CBI therapies such as anti-PD(L)1/anti-TIGIT have been tested and demonstrate significant promise in clinical trials⁸⁻¹¹ Another approach is the development of armored or immune restoring CAR T cells that are engineered to secrete an immunomodulatory payload directly at the tumor site, increasing the efficacy while decreasing the on-target/off-tumor side effects seen with systemic delivery^{12,13}.

In addition to T cells, various immune cells have been utilized for the creation of new CARs, including natural killer cells (NK-CAR) and macrophages (CAR-M) and what unites these cells is that they provide direct anti-tumor activityl^{4,15}. A critical component of humoral immunity, B cells produce the antibodies on which the field of immunotherapy was originally developed. However, they possess no intrinsic cytotoxic capabilities and thus have been largely excluded from these advancements.

Without wishing to be bound by theory, the Chimeric Antibody Secreting and Signaling (CASS) B cell platform is a unique B cell based cellular therapy that does not rely upon direct cytotoxicity but utilizes two intrinsic capabilities of B cells; the ability to secrete high levels of CBI antibodies to reverse the immunosuppressive TME and the ability to process and present antigens on MHC class II molecules, resulting in the recruitment of CD4+ T cells and allowing for enhanced tumor cell recognition and neoantigen spreading. While inducible, targeted delivery of the CBI will decrease irAEs, the ability to serve as a professional APC makes the CASS B cell platform unique in the cell therapy space, as CASS B cells have the power to initiate a robust anti-tumor response. The impact of this is further exemplified by MHC class II neoantigens that play a key function in innate anti-tumor responses^{16,17}.

Described herein are MSLN+ targeted CASS B cells secreting an anti-PD1/TIGIT bsAb for NSCLC, the modular design of CASS B cells allow for easy targeting of other TAAs and the secreted payload can be adjusted to target the relevant immunologic axis, allowing for the adaptation of CASS B cells to a wide variety of cancers. As B cells are long lived and an important part of immunologic memory, CASS B cells continuously deliver the therapeutic payload at the tumor and after the primary tumor has been eradicated, provides a lifelong immunosurveillance system against metastasis and reoccurrence. Without wishing to be bound by theory, the long duration and inducible nature of the CASS B cell platform can be adapted to other diseases treated with biologic therapies, such as cardiovascular and autoimmune diseases and neurological disorders, which can require long term disease maintenance and rapid administration of therapeutics to treat acute symptoms.

Labs have worked in the development of armored and immune restoring CAR T cells, including those that secrete cytokines, CBI mAbs, and bsAbs (BiTE) with the goal of enhancing CAR T efficacy by remodeling the local TME^{12,13}. Studies using inducible or constitutively expressed payloads have demonstrated that a) engagement of bystander immune cells and recruitment of other anti-cancer cell types significantly enhances therapeutic efficacy and b) proteins secreted at the tumor site remain at high concentration around the tumor with minimal transfer to serum¹⁸⁻²⁰.

B cell engineering is a more recent accomplishment and has mainly focused on creating B cells that secrete neutralizing, anti-pathogenic antibodies against RSV and HIV²¹⁻²³. Unlike the cancer therapeutic described herein, the B cells described in these works focus on systemic production of antibodies to neutralize viral infection and utilize CRISPR/Cas9 to insert the recombinant mAb into the Ig locus of the B cell. This has the added benefit of allowing the antibody to continue to undergo affinity maturation, a requirement for combating infectious diseases but not for targeting immune markers. Without wishing to be bound by theory, we will manipulate a B cell to express an engineered antibody of choice in an inducible manor and that upon activation, these engineered B cells not only differentiate into Ab secreting cells, but also memory B cells providing long-term protection²⁴.

### Non-Limiting, Exemplary Research Plan

Aim 1: Engineering and optimization of CASS B cell constructs—The appropriate scFvs (anti-MSLN, PD1, TIGIT) will be selected for CASS B cell development followed by optimization of the signaling domains and inducible response element²⁵⁻²⁸ Protocols for high efficiency transduction and exponential expansion of primary B cells will also be optimized utilizing various lentivirus envelope proteins and culture conditions²⁹⁻³².

### Experimental Design and Procedures

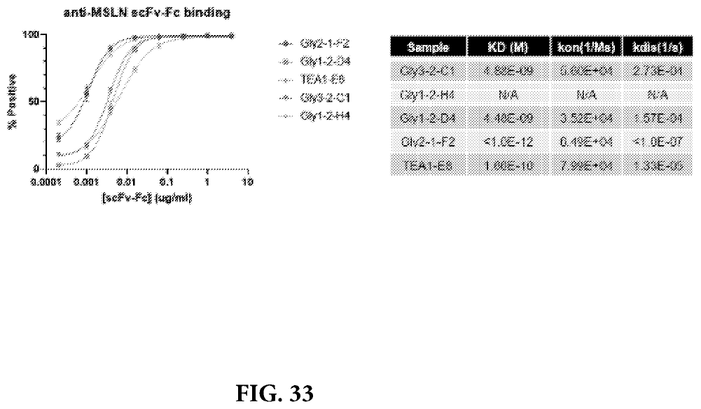

Antibody discovery, engineering, and optimization: we have built a 27-billion member human phage library that has been used to isolate a number of therapeutic antibodies³³⁻³⁷. Preliminary sets of anti-PD1, TIGIT, and MSLN (FIG. 33) have been identified for further engineering and optimization.

Bispecific antibody design and engineering: As PD1/TIGIT are expressed on immune cells, immunodepletion is not preferred^{38,39}. The current design utilizes a tandem scFv construct previously developed and characterized, however other designs will be considered.

B cell isolation, expansion, and transduction: B cells will be isolated and various expansion media formulations tested^{23,40,41}. For transduction, B cells will be activated, transduced, and sorted 72 hours post transduction.

Generation of IgG-BCR construct: Our engineered IgG-BCR is built using an scFv fused to a membrane bound IgG1 hinge-Fc that expresses at high levels and binds the target antigen⁴². Initial transduction experiments using primary B cells demonstrate high titer transduction for multiple donors and DNA constructs. We have previously utilized a NFAT/NFkB RE to develop an inducible T cell activation assay. For optimization experiments, a fluorescent protein will be used in place of the secreted bsAb.

Without wishing to be bound by theory, there will not be problems with mAb discovery or CASS B cell design. mAb engineering techniques employed by our lab will allow us to develop the panel of mAbs previously discovered to find a lead candidate for each target. Without wishing to be bound by theory, we can achieve high transduction efficiency and exponential expansion of transduced cells for in vivo experiments.

We can identify a lead antibody for each target. We can then construct the vector and successful transduction/expansion of CASS B cells.

Aim 2: In vitro testing and efficacy of CASS B cells-In-depth in vitro characterization will be performed to identify activation thresholds and quantify bsAb secretion levels. Final in vitro assays will be performed using patient-derived organotypic tumor spheroids (PDOTS), allowing us to observe CASS B cell homing and perform detailed analysis of the bsAb payload.

### Non-Limiting, Exemplary Experimental Design and Procedures

CASS B cell activation assays: Supernatant from activated CASS B cells will be screened via ELISA to measure bsAb and cytokine concentration.

NSCLC PDOT generation and evaluation: Generation of NSCLC PDOTs will be performed following the protocol outlined in Jenkins et al and Aref et al^{43,44}. In addition to testing bsAb and CASS B cell efficacy, a comparative experiment will be performed to validate therapeutic differences between CASS B cell and CAR T cell treatments. Immune profiling via IHC, scRNAseq and cytokine profiling will be performed. Due to the limited availability of NSCLC tumor tissue, a backup plan has been devised to use mesothelioma tumors for PDOT generation. Mesothelioma has a highly suppressive TME and immune infiltrating cells display high levels of exhaustion markers, making this an ideal alternative for in vitro assays⁴⁵⁻⁴⁸.

Projected results: Aim 2 will provide an in-depth understanding of CASS B cell activation and validate an optimal anti-MSLN scFv for selective targeting of MSLN+ tumors. PDOTs will provide a comprehensive data set of CASS B cell efficacy via cytokine secretion and transcriptional profiles, and that anti-MSLN CASS B cells will traffic to the tumor and the secreted bsAb will reverse the suppression of immune cells. Additionally, we will see greater epitope spreading and bystander immune cell activation with the CASS B cell compared to CAR T cell therapy.

Milestones: The first milestone in Aim 2 will be the generation of activation curves for CASS B cells and quantification of bsAb secretion. The next milestone will be establishment of the PDOTS, and efficacy testing of the bsAb systemically and as a CASS B cell payload. Another milestone will be comparing CASS B cell and CAR T cell therapy and the generation of immune profiles for each therapy via IHC and scRNAseq.

Aim 3: In vivo efficacy using HLA matched, humanized NSCLC mouse models—in vivo experiments will be performed on a cell line derived xenograft (CDX) model in humanized mice. The use of a validated patient derived xenograft (PDX) model from a publicly available repository for final testing will improve tumor integrity and phenotypic characteristics while accurately mimicking the TME of in vivo tumors.

### Experimental Design and Procedures

Establishment of an NSCLC CDX and PDX model in humanized mice: NSCLC cell lines and PDX models will be screened for PDL1 and CD155 expression levels prior to luciferization⁴⁹. Human immune system reconstitution will be performed as reported by ourselves and others⁵⁰⁻⁵². To generate growth curves, varying concentrations of NSCLC cell lines/PDX tumors will be implanted into the rear flank of a humanized mouse⁵³.

Assessment of CASS B cell efficacy in humanized NSCLC mouse model: We will use the CDX and PDX models to validate the efficacy of the bsAb and CASS B cell in vivo. Samples will be analyzed by multiparameter FACS, IHC/ISH, and scRNASeq following established protocols regularly used in our lab⁵⁴⁻⁶³.

Without wishing to be bound by theory, there will be successful NSCLC model generation. These models will demonstrate that CASS B cells cluster around the tumor and secrete high levels of bsAb, decreasing the immunosuppressive nature of the TME and recruiting additional anti-tumor immune cells. IHC/ISH and scRNAseq will provide molecular evidence of bsAb efficacy, CASS B cell homing, and activation of APC pathways in both CASS B cells and CD4 T cells, while 5′ scRNAseq of the TCR/BCRs will be used to directly monitor epitope spreading among TILs.

Statistical considerations: With 5 animals per group for individual pair-wise comparisons between conditions of interest and different outcome measures we have power of 0.93 to detect a difference in the means equal to 2.5SD using two-sample two-tailed t-test at 0.05 level.

Milestones: The first milestone in Aim 3 will be the generation of CDX and PDX models for NSCLC. Subsequent milestones can be initiation and completion of the planned animal experiments testing the bsAb in a CDX model, and the CASS B cell in CDX and PDX models. Due to the wealth of data generated by these experiments, tertiary milestones can be completion of data analysis for each animal experiment.

### REFERENCES CITED IN THIS EXAMPLE

1. Ho, M. et al. Mesothelin expression in human lung cancer. Clin. Cancer Res. 13, 1571-1575 (2007).
2. Thomas, A. et al. High mesothelin expression in advanced lung adenocarcinoma is associated with KRAS mutations and a poor prognosis. Oncotarget 6, 11694-11703 (2015).
3. Weidemann, S. et al. Mesothelin Expression in Human Tumors: A Tissue Microarray Study on 12,679 Tumors. Biomedicines 9, 4-9 (2021).
4. Berghmans, T., Durieux, V., Hendriks, L. E. L. & Dingemans, A.-M. Immunotherapy: From Advanced NSCLC to Early Stages, an Evolving Concept. Front. Med. 7, 1-16 (2020).
5. Qu, J. et al. The progress and challenge of anti-PD-1/PD-L1 immunotherapy in treating non-small cell lung cancer. Ther. Adv. Med. Oncol. 13, 1-28 (2021).
6. Ott, P. A. & Wu, C. J. Cancer vaccines: Steering t cells down the right path to eradicate tumors. Cancer Discov. 9, 476-481 (2019).
7. Rogado, J. et al. Immune-related adverse events predict the therapeutic efficacy of anti-PD-1 antibodies in cancer patients. Eur. J. Cancer 109, 21-27 (2019).
8. Lee, D. H. Update of early phase clinical trials in cancer immunotherapy. BMB Rep. 54, 70-88 (2021).
9. Ahn, M.-J. et al. 1400P Vibostolimab, an anti-TIGIT antibody, as monotherapy and in combination with pembrolizumab in anti-PD-1/PD-L1-refractory NSCLC. Ann. Oncol. 31, S887 (2020).
10. Niu, J. et al. 1410P Safety and efficacy of vibostolimab, an anti-TIGIT antibody, plus pembrolizumab in patients with anti-PD-1/PD-L1-naive NSCLC. Ann. Oncol. 31, S891-S892 (2020).
11. Rodriguez-Abreu, D. et al. Primary analysis of a randomized, double-blind, phase II study of the anti-TIGIT antibody tiragolumab (tira) plus atezolizumab (atezo) versus placebo plus atezo as first-line (1L) treatment in patients with PD-L1-selected NSCLC (CITYSCAPE). J. Clin. Oncol. 38, 9503 (2020).
12. Suarez, E. R. et al. Chimeric antigen receptor T cells secreting anti-PD-L1 antibodies more effectively regress renal cell carcinoma in a humanized mouse model. Oncotarget 7, (2016).
13. Hawkins, E. R., D'souza, R. R. & Klampatsa, A. Armored CAR T-cells: The next chapter in T-cell cancer immunotherapy. Biol. Targets Ther. 15, 95-105 (2021).
14. Mukhopadhyay, M. Macrophages enter CAR immunotherapy. Nat. Methods 17, 561 (2020).
15. Oelsner, S. et al. Continuously expanding CAR NK-92 cells display selective cytotoxicity against B-cell leukemia and lymphoma. Cytotherapy 19, 235-249 (2017).
16. Kreiter, S. et al. Mutant MHC class II epitopes drive therapeutic immune responses to cancer. Nature 520, 692-696 (2015).
17. Alspach, E. et al. MHC-II neoantigens shape tumour immunity and response to immunotherapy. Nature 574, 696-701 (2019).
18. Chmielewski, M., Kopecky, C., Hombach, A. A. & Abken, H. IL-12 release by engineered T cells expressing chimeric antigen receptors can effectively muster an antigen-independent macrophage response on tumor cells that have shut down tumor antigen expression. Cancer Res. 71, 5697-5706 (2011).
19. Rafiq, S. et al. Targeted delivery of a PD-1-blocking scFV by CAR-T cells enhances anti-tumor efficacy in vivo. Nat. Biotechnol. 36, 847-858 (2018).
20. Iwahori, K. et al. Engager T cells: A new class of antigen-specific t cells that redirect bystander T cells. Mol. Ther. 23, 171-178 (2015).
21. Voss, J. E. et al. Reprogramming the antigen specificity of b cells using genome-editing technologies. Elife 8, 1-22 (2019).
22. Nahmad, A. D. et al. Engineered B cells expressing an anti-HIV antibody enable memory retention, isotype switching and clonal expansion. Nat. Commun. 11, 1-10 (2020).
23. Moffett, H. F. et al. B cells engineered to express pathogen-specific antibodies protect against infection. Sci. Immunol. 4, (2019).
24. Nahmad, A. D. et al. Engineered B cells expressing an anti-HIV antibody enable memory retention, isotype switching and clonal expansion. Nat. Commun. 11, 1-10 (2020).
25. Uchibori, R. et al. Functional Analysis of an Inducible Promoter Driven by Activation Signals from a Chimeric Antigen Receptor. Mol. Ther.-Oncolytics 12, 16-25 (2019).
26. Zhang, L. et al. Improving adoptive T cell therapy by targeting and controlling IL-12 expression to the tumor environment. Mol. Ther. 19, 751-759 (2011).
27. Hooijberg, E., Bakker, A. Q., Ruizendaal, J. J. & Spits, H. NFAT-controlled expression of GFP permits visualization and isolation of antigen-stimulated primary human T cells. Blood 96, 459-466 (2000).
28. Lin, T. et al. Establishment of NF-κB sensing and interleukin-4 secreting mesenchymal stromal cells as an “on-demand” drug delivery system to modulate inflammation. Cytotherapy 19, 1025-1034 (2017).
29. Girard-Gagnepain, A. et al. Baboon envelope pseudotyped LVs outperform VSV-G-LVs for gene transfer into early-cytokine-stimulated and resting HSCs. Blood 124, 1221-1231 (2014).
30. Tomás, H. A. et al. Improved GaLV-TR Glycoproteins to Pseudotype Lentiviral Vectors: Impact of Viral Protease Activity in the Production of LV Pseudotypes. Mol. Ther.-Methods Clin. Dev. 15, 1-8 (2019).
31. Bernadin, O. et al. Baboon envelope LVs efficiently transduced human adult, fetal, and progenitor T cells and corrected SCID-X1 T-cell deficiency. Blood Adv. 3, 461-475 (2019).
32. Winiarska, M. et al. Selection of an optimal promoter for gene transfer in normal B cells. Mol. Med. Rep. 16, 3041-3048 (2017).
33. Xu, C. et al. Unique biological properties of catalytic domain directed human anti-CAIX antibodies discovered through phage-display technology. PLOS One 5, (2010).
34. Sui, J. et al. Structural and functional bases for broad-spectrum neutralization of avian and human influenza A viruses. Nat. Struct. Mol. Biol. 16, 265-273 (2009).
35. Fu, Y. et al. A broadly neutralizing anti-influenza antibody reveals ongoing capacity of haemagglutinin-specific memory B cells to evolve. Nat. Commun. 7, (2016).
36. Chang, D.-K. et al. Humanization of an anti-CCR4 antibody that kills cutaneous T-cell lymphoma cells and abrogates suppression by T-regulatory cells. Mol. Cancer Ther. 11, 2451-2461 (2012).
37. Chang, D. K. et al. Humanized mouse G6 anti-idiotypic monoclonal antibody has therapeutic potential against IGHV1-69 germline gene-based B-CLL. MAbs 8, 787-798 (2016).
38. Marin-Acevedo, J. A. et al. Next generation of immune checkpoint therapy in cancer: New developments and challenges. J. Hematol. Oncol. 11, 1-20 (2018).
39. Saunders, K. O. Conceptual approaches to modulating antibody effector functions and circulation half-life. Front. Immunol. 10, 1-20 (2019).
40. Su, K.-Y., Watanabe, A., Yeh, C.-H., Kelsoe, G. & Kuraoka, M. Efficient Culture of Human Naive and Memory B Cells for Use as APCs. J. Immunol. 197, 4163-4176 (2016).
41. Kuraoka, M. et al. Complex Antigens Drive Permissive Clonal Selection in Germinal Centers. Immunity 44, 542-552 (2016).
42. Wan, Z. et al. The activation of IgM- or isotype-switched IgG- and IgE-BCR exhibits distinct mechanical force sensitivity and threshold. Elife 4, 1-24 (2015).
43. Aref, A. R. et al. 3D microfluidic: Ex vivo culture of organotypic tumor spheroids to model immune checkpoint blockade. Lab Chip 18, 3129-3143 (2018).
44. Jenkins, R. W. et al. Ex Vivo Profiling of PD-1 Blockade Using Organotypic Tumor Spheroids. Cancer Discov. 176, 139-148 (2018).
45. Lv, J. & Li, P. Mesothelin as a biomarker for targeted therapy. Biomark. Res. 7, (2019).
46. Marcq, E. et al. Abundant expression of TIM-3, LAG-3, PD-1 and PD-L1 as immunotherapy checkpoint targets in effusions of mesothelioma patients. Oncotarget 8, 89722-89735 (2017).
47. Klampatsa, A. et al. Phenotypic and functional analysis of malignant mesothelioma tumor-infiltrating lymphocytes. Oncoimmunology 8, 1-12 (2019).
48. Alay, A. et al. Integrative transcriptome analysis of malignant pleural mesothelioma reveals a clinically relevant immune-based classification. J. Immunother. Cancer 9, (2021).
49. Liu, J. F. et al. Establishment of patient-derived tumor xenograft models of epithelial ovarian cancer for preclinical evaluation of novel therapeutics. Clin. Cancer Res. 23, 1263-1273 (2017).
50. Biswas, S. et al. Humoral immune responses in humanized BLT mice immunized with West Nile virus and HIV-1 envelope proteins are largely mediated via human CD5+B cells. Immunology 134, 419-433 (2011).
51. Chang, H. et al. Human B-cell ontogeny in humanized NOD/SCID γc(null) mice generates a diverse yet auto/poly- and HIV-1-reactive antibody repertoire. Genes Immun. 13, 399-410 (2012).
52. Billerbeck, E. et al. Development of human CD4+FoxP3+ regulatory T cells in human stem cell factor-, granulocyte-macrophage colony-stimulating factor-, and interleukin-3-expressing NOD-SCID IL2Rγnull humanized mice. Blood 117, 3076-3086 (2011).
53. Lin, S. et al. Establishment of peripheral blood mononuclear cell-derived humanized lung cancer mouse models for studying efficacy of PD-L1/PD-1 targeted immunotherapy. MAbs 10, 1301-1311 (2018).
54. Stoeckius, M. et al. Cell Hashing with barcoded antibodies enables multiplexing and doublet detection for single cell genomics. Genome Biol. 19, 1-12 (2018).
55. Tian, L. et al. scPipe: A flexible R/Bioconductor preprocessing pipeline for single-cell RNA-sequencing data. PLOS Comput. Biol. 14, 1-15 (2018).
56. Amezquita, R. A. et al. Orchestrating single-cell analysis with Bioconductor. Nat. Methods 17, 137-145 (2020).
57. Hao, Y. et al. Integrated analysis of multimodal single-cell data. Cell 184, 3573-3587.e29 (2021).
58. Lytal, N., Ran, D. & An, L. Normalization Methods on Single-Cell RNA-seq Data: An Empirical Survey. Front. Genet. 11, 1-14 (2020).
59. McCarthy, D. J., Campbell, K. R., Lun, A. T. L. & Wills, Q. F. Scater: Pre-processing, quality control, normalization and visualization of single-cell RNA-seq data in R. Bioinformatics 33, 1179-1186 (2017).
60. Haghverdi, L., Lun, A. T. L., Morgan, M. D. & Marioni, J. C. Batch effects in single-cell RNA-sequencing data are corrected by matching mutual nearest neighbors. Nat. Biotechnol. 36, 421-427 (2018).
61. Chen, Y. et al. edgeR: differential analysis of sequence read count data User's Guide. R Packag. 1-121 (2020).
62. Subramanian, A. et al. Gene set enrichment analysis: A knowledge-based approach for interpreting genome-wide expression profiles. Proc. Natl. Acad. Sci. U.S.A. 102, 15545-15550 (2005).
63. DeTomaso, D. et al. Functional interpretation of single cell similarity maps. Nat. Commun. 10, (2019).

### Example 5

### Design and Testing of Dual-Targeted Fine-Tuned Immune Restoring (DFIR) CAR-T Cell Therapy to Achieve Cures of High Grade Serous Ovarian Cancer

Ovarian cancer (OvCA) is the fifth leading cause of cancer deaths in American women. Globally, there are 200,000 new diagnoses and 120-130,000 deaths from the disease each year1. Due to the absence of specific symptoms or signs and the lack of trustworthy screening for early detection, the majority of women with OvCA (60-65%) are diagnosed at a late stage when the cancer has spread beyond the confines of the ovary. Currently, the standard treatment of OvCA is surgical intervention followed by platinum-containing chemotherapy, such as carboplatin plus paclitaxel 2,3. Treatment for stage III or IV disease is rarely curative, with 5-year survival rates under 20%. No effective therapy is available for relapsed or metastatic disease that has failed first-line therapy 4. In addition, several recent clinical trials of anti-PD (L) 1 immune checkpoint inhibitors (ICI) without and with chemotherapy have shown poor response rates 5. There is an urgent need for more effective therapies.

Chimeric Antigen Receptor (CAR) T cell therapy is a new type of “living drug”. The CAR contains a single chain variable antibody fragment (scFv) linked to an intracellular signaling block that includes a CD3-ζ (z) activation domain (1st generation), with a CD28 or 41BB costimulatory domain (2nd generation), or both CD3-ζ and a costimulatory domain (3rd generation) 6. The FDA approval of 2nd generation CAR-T cell therapies have ushered this new type of cellular immunotherapy into mainstream cancer therapy 7 for hematologic malignancies 8-10. However, these results have not been readily translatable to solid tumors 11. Indeed, several major hurdles have been recognized that limit our ability to achieve “cures” of solid tumors using CAR-T cells. These include on-target, off-tumor toxicity due to sharing of tumor associated antigen (TAA) epitopes on normal tissues, tumor heterogeneity, and the immunosuppressive tumor microenvironment (TME). To address these issues, we have constructed Dual-targeted, Fine-tuned, Immune Restoring (DFIR) CAR-T cells for the treatment of solid tumors with an emphasis on improving anti-tumor efficacy and patient safety.

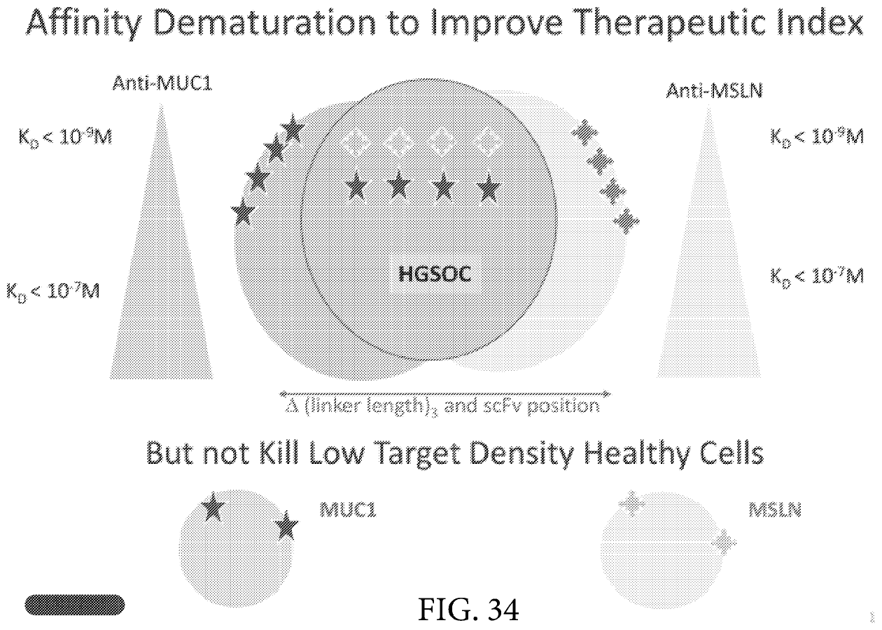

For this DoD ovarian cancer (OvCA) research program pilot award we will validate DFIR CAR-T therapy against high grade serous ovarian cancer (HGSOC). Specifically, we will engineer an anti-mucin 1 (MUC1)/anti-mesothelin (MSLN) dual-targeted CAR which serves to increase efficacy by allowing CAR-T activation with the presence of MUC1 or MSLN to account for solid tumor heterogeneity and to mitigate against single antigen escape. 12,13. Because many antigens used to target epithelial tumors are also present at low density on healthy tissue, it is important to establish an increased safety profile for the CAR-T cells. Elevated safety is addressed though fine-tuned CARs such that the affinities of the scFv targeting moieties are tailored to only recognize high density tumor TAAs but not the same antigens expressed as physiologic levels on normal tissues (FIG. 34). Immune restoration will occur through localized secretion of a bispecific antibody (BsAb) at the tumor site that serves as a potent checkpoint blockade inhibitor (ICI) and inhibitor of regulatory T-cell (Treg) suppression and recruitment 14. For this project the first arm of the BsAb is directed against TIGIT which has been shown to be over expressed on tumor antigen-specific (TA-specific) CD8+ T cells and CD8+ tumor infiltrating lymphocytes (TILs) from individuals with HGSOC. The second arm of the BsAb is targeted to CCR4 which is overexpressed on regulatory T cells (Tregs) and are elevated in the tumor infiltrating lymphocyte population in OvCA 15-18.

Here, we will validate that the antitumor efficacy and safety of CAR-T cell therapy for HGSOC can be greatly improved through dual-targeting and fine-tuning of the binding affinity of the anti-MUC1 and anti-MSLN CAR targeting moieties. We will further validate that the CAR-T cells can be additionally engineered to secrete a bispecific antibody (BsAb) that targets two of the major immunosuppressive pathways that are operative in HGSOC. The locally secreted BsAb will be directed to TIGIT which is present on exhausted T cells, Tregs and NK cells and CCR4, which is upregulated on Tregs and plays a key role in chemotaxis. Without wishing to be bound by theory, simultaneously blocking these two pathways will reverse immunosuppression and restore local anti-tumor immunity.

### Specific Aims:

Aim 1-Validate OvCA cell killing efficacy by anti-MUC1/MSLN DFIR CAR-T cells that constitutively secrete anti-CCR4/TIGIT BsAb locally at the tumor site using in vitro studies. Subtasks will include validation of reversal of T-cell exhaustion and restoration of cytokine secretion.

DFIR CAR-T cells will be designed to have high killing efficacy of single antigen positive OvCA cells (MUC1 or MSLN) and double positive (MUC1 and MSLN) which we term “and/or” gating. Fine-tuning of the binding affinities will be achieved by validating different affinity anti-MUC1 and anti-MSLN scFvs as mono-CARs for ability to only kill tumor cells that overexpress either of these two antigens but not healthy cells that can express physiologic (lower) levels of these antigens.

DFIR CAR-T cell antibody payloads have been determined by mining scRNAseq data from publicly available HGSOC patient samples to identify “public” signatures of two nonoverlapping dysfunctional pathways of tumor infiltrating immune cells that we can use for therapeutic intervention.

Patient-derived organotypic tumor spheroids (PDOTS) will be used to both assess CAR-T cell killing alone compared to CAR-T cells that secrete antibodies that act as ICIs. The results from these ex vivo 3D cultures with TME will guide in vivo DFIR CAR-mouse therapeutic models.

Antibody engineering techniques will allow us to fine-tune the affinity of the MUC1 and MSLN scFv targeting moieties to achieve high efficacy tumor cell killing.

Design of new anti-CCR4/TIGIT BsAb will serve as a checkpoint control inhibitor of the TIGIT/CD155 axes on Tregs, cytotoxic T cells and NK cells, and as an antagonist against chemotaxis of Tregs to tumor cell that secrete the chemokines CCL17/CCL22.

Improved CAR Efficacy and Safety: Solid tumors can show phenotypic and genotypic heterogeneity. This is due to inherent properties effecting critical pathways such as DNA repair and replication that lead to uncontrolled tumor growth. In addition, immune editing can be seen and results in neutralization escape that can lead to the outgrowth of single positive tumor cells. This can lead to treatment failures if CAR-T cells are directed to a single target. Without wishing to be bound by theory, dual-targeting can provide an “and/or” gating strategy whereby CAR-T cells can target and kill singly positive tumor cells and/or double positive with the latter showing synergistic binding and killing due to increased avidity 19-21.

We have approached the issue of dual-targeting by isolating human anti-MUC1 scFvs that are directed to the membrane proximal SEA domain. We panned our 27×109 member human scFv phage display library against recombinant MUC1-SEA polypeptide and identified a lead scFv (T4E3). We have also received a second anti-MUC1-SEA scFv (3D1) that targets a similar domain on MUC1. The binding affinity of T4E3 scFvFc (5.26) is circa 10-fold higher than 3D1 scFvFc (57.02 nM) thereby providing us with a good therapeutic range to evaluate the effects of binding affinity and MUC1 target density on tumor cell killing as well as on-target off-tumor killing of healthy cells expressing low density of MUC-1 shows efficient killing of MUC1+COV263 cells (bottom) even at low E:T (0.8:1) ratios (% survival at 48 hrs) whereas control MUC1-HCT116v cells (top) were not killed.

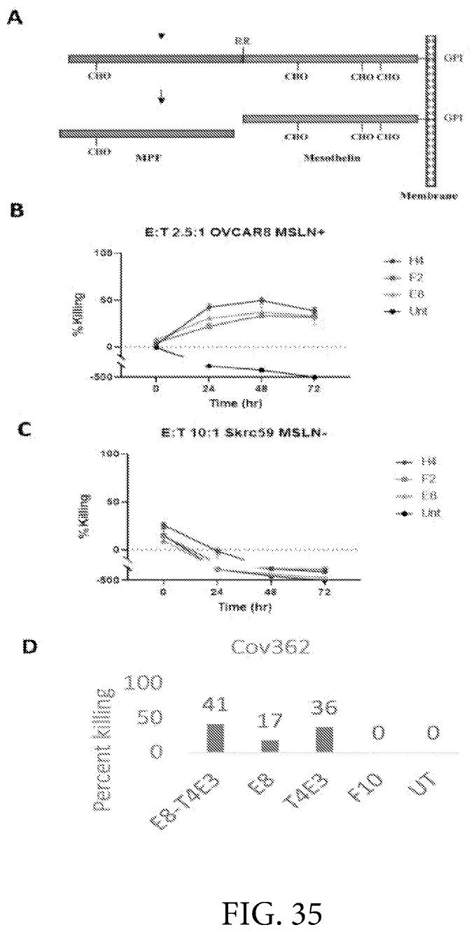

A similar discovery campaign was performed to identify anti-MSLN scFvs. To accommodate the conformational change that MSLN undergoes upon release from the GPI linker, we utilized a series of MSLN+ cells in a whole cell panning approach to isolate new anti-MSLN antibodies from our phage library (FIG. 35, panel A). This campaign resulted in the identification of several anti-MSLN scFvs that had potent (FIG. 35, panel B) and specific killing of MSLN+ OVCAR8 cells but not MSLN-SK59 renal cell carcinoma cells (FIG. 35, panel C). Importantly, each MUC1 (T4E3) and MSLN (E8) CAR was able to kill double MUC1+, MSLN+ CoV362 cells as was the dual targeted T4E3-E8 CAR (FIG. 35, panel D).

IR CAR-T cells change the TME: Changing the TME is critically important for CAR-T cell therapy to both reverse immune cell exhaustion at the tumor site and to prevent the newly arrived CAR-T cells at the tumor site from becoming exhausted. We have spent considerable effort in analyzing published scRNAseq data to identify the targets for which we can develop a BsAb that can act as a potent ICI and reverse immunosuppression at the tumor site. We have completed these discovery efforts and two targets, TIGIT and CCR4 were chosen. Binding data for anti-TIGIT mAbs and their competition with its immunosuppressing ligand CD155 was performed. This Ab mediated blockade of CD155 binding to TIGIT can block the immunosuppression through this axis.

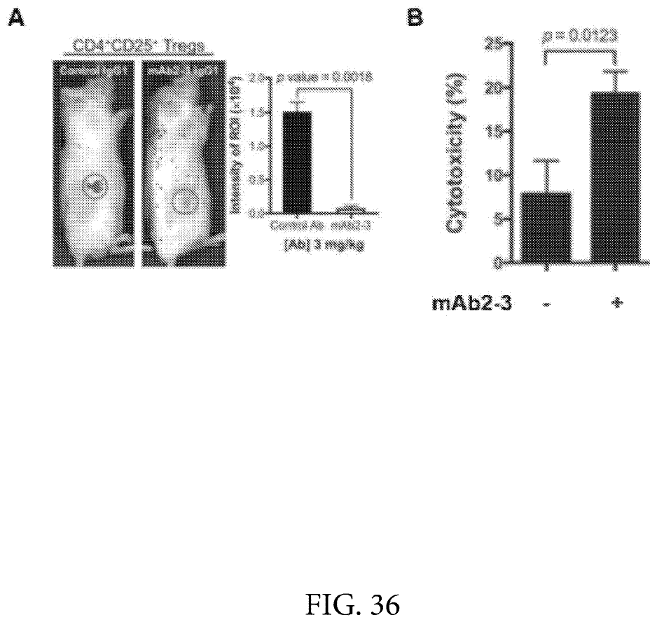

CCR4 is a chemokine receptor present on immunosuppressive Tregs that migrate to the OvCA tumor site. Our lead anti-CCR4 antibody is mAb2-322-24. This antibody blocks the in vivo migration of human Tregs to the OvCA cells that secrete chemokines CCL17 and CCL22 (FIG. 36, panel A) and restores in vivo tumor cell killing (FIG. 36, panel B)

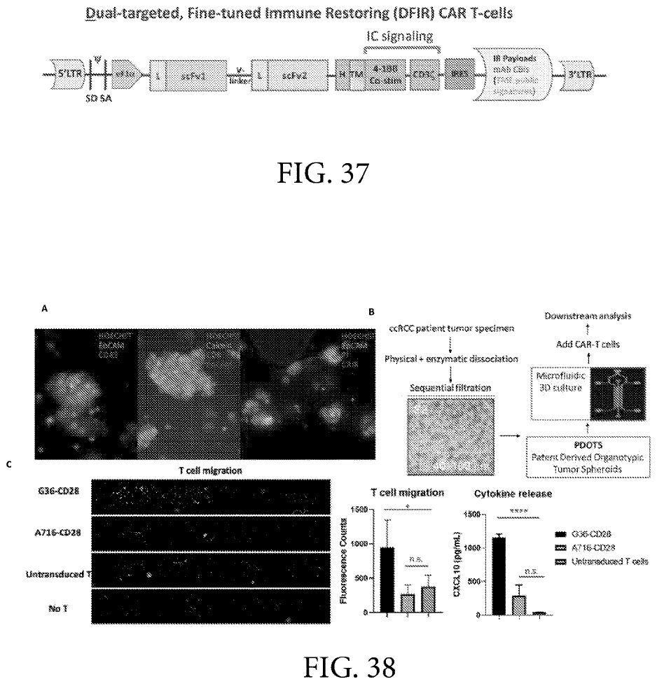

Research plan: We will validate individual anti-MUC1 and anti-MSLN CARs for killing against OVCAR8, CoV362 and MUC1 or MSLN transduced 293 T cells We will perform additional in vitro CAR-T cell killing studies of OVCAR8 and CoV362 cells that express different densities of MUC1 and MSLN as well as negative control MUC1-MSLN-SK59 cells and other low density MSLN+ or MUC1+ cell lines. We will validate killing activity using our Celigo assay as published 25. The results of these studies will be analyzed for ability to kill tumor cells with different MUC1 and MSLN densities and safety will be analyzed for lack of killing of the control cell lines. These studies will allow us to validate the anti-MUC1 and anti-MSLN scFvs that will be used for construction of dual-targeted CARs. Next, DF CAR-T cells will be constructed using the lead anti-MUC1 and anti-MSLN scFvs in both orientations (proximal versus distal to T cell membrane) and different inter-scFv linker lengths (GGGGS) 1-3 (FIG. 34 and FIG. 37). This will ensure that the orientation and accessibility of each scFv to its target is optimized. From these studies one lead dual-targeted CAR will be chosen for further testing.

Delivery of antibody payloads will involve a different set of studies that use primary OvCA PDOTs. We have successfully established ccRCC ex vivo 3D models which recapitulate the TME and provides a facile means to screen the efficacy of CAR-T variants (FIG. 38, panel A). The workflow is shown in FIG. 38, panel B 26. We demonstrated that ccRCC PDOTS serve as a powerful tool to assess both efficacy and migration of CAR-T cells. Anti-CAIX G36 CAR-T cells significantly migrated from side channel to the middle channel with ccRCC spheroids and secreted CXCL10 after 6-days co-incubation (FIG. 38, panel C). For our OvCA studies, we will transduce CAR lentiviruses into healthy donor T cells at CD4:CD8 ratio of 2:1. Following ex vivo purification and expansion, we will add DFIR CAR-T cells to the outer wells of the device and test the migration and cytokine secretion of lead DFIR CAR-T cells secreting anti-TIGIT, anti-CCR4 or anti-CCR4/TIGIT BsAb. CAR-T migration will be measured by co-transducing CAR-T cells with lentivirus expressing ZsGreen and viability by PI staining. Cytokine release profiles will be measured by Luminex.

We will validate these mono CAR-T cells and DFIR CAR-T cells with different payloads by harvesting DFIR CAR-T/TILs and supernatants from the microfluidic devices as reported 26. We will validate whether there are phenotypic and functional properties that distinguish anti-MUC1/MSLN DFIR CAR-T cells secreting anti-CCR4/TIGIT BsAbs from other treatments and whether there are differences between the HGSOC cell donors.

Results: Without wishing to be bound by theory, anti-MUC1/MSLN DF CAR-T cells can be engineered to selectively kill OvCA cells that overexpress MUC1 and/or MSLN but not healthy cells that express physiologic (low) levels or are negative for these TAAs. Further, anti-MUC1/MSLN DFIR CAR-T cells that secrete anti-CCR4/TIGIT BsAbs will show enhanced killing compared to anti-MUC1/MSLN DF CAR-T cells that do not secrete ICI payloads. Moreover, DFIR CAR-T cells that secrete anti-CCR4/TIGIT BsAbs will show greater reversal/prevention of TIL/CAR-T exhaustion than DFIR CAR-T cells that secrete only anti-TIGIT or anti-CCR4 Abs because of dual ICI pathway blockade. The mechanism(s) of action of our BsAb secreting DFIR CAR-T cells will be by both preventing CAR-T cell exhaustion and restoring anti-tumor immunity to the TILs. Prevention/reversal of CAR-T/TIL exhaustion will be quantified by FACS staining for PD1, TIM3 and LAG3 as well as determining their levels of cytokine secretion (e.g IFNγ, IL-2).

Data analysis and statistical considerations: Each CAR-T assay will be performed in triplicate and will be repeated 3 times with three PBMC donors and the killing percentage will be summarized across these conditions by the sample mean. We will first compare anti-MUC1 and anti-MSLN mono CAR-T cells for specific (efficacy) and background (safety) killing by examining 2-3 different CARs with differing affinities against each target and against 2-3 OvCA cell lines with different MUC1 and MSLN target densities. From these studies we will identify an anti-MUC1 and anti-MLSN CAR to be used, for example selecting the CAR which has greatest efficacy across the cell lines while maintaining acceptable safety of at most 10% background killing, to create our dual-targeted anti-MUC1/MSLN DFIR CAR. The effects of antibody payload secretion on activation of DFIR CAR-T cells will be monitored by functional outcomes in PDOT studies. The outcomes to be monitored by FACS include reversal of upregulated exhaustion markers (PD1, TIM3, LAG3) in TIL/CAR-T cells and increase in TIL/CAR-T survival and by Luminex, increase in pro-inflammatory cytokine secretion.

In vitro CAR-T killing: Data will be analyzed in GraphPad Prism and two-way ANOVA accounting for multiple comparisons will be used for statistical analysis.

### Cytokine ELISA: Data Will be Analyzed in GraphPad Prism and Two-Way ANOVA Accounting for Multiple Comparisons Will be Used for Statistical Analysis

Aim 2-Perform in vivo studies validating CAR-T cell efficacy in humanized mice (NSG-SGM3) bearing HLA matched ovarian tumors. Studies will be designed with appropriate control groups to separately assess the effects of anti-MUC1/anti-MSLN DFIR CAR-T cells and their anti-CCR4/TIGIT BsAb payloads. Subtasks will include analysis of TILs by scRNASeq, multiparameter FACS analysis and cytokine secretion profiles from plasma.

### Develop Humanized Mouse Model to Interrogate the OvCA TME and DFIR CAR-T Interventions

Use scRNAseq to Catalogue and Quantitate Changes in TME that Result from DFIR CAR-T Therapy

Rationale and data: Without wishing to be bound by theory, changing the TME in HGSOC can be achieved through combined checkpoint blockade inhibition and by blocking the recruitment of Tregs to the tumor site. We have demonstrated marked inhibition of Treg migration to the OvCA tumor site in vivo by our anti-CCR4 antibody that also resulted in marked inhibition of tumor cell growth. We have demonstrated the anti-tumor efficacy for an IR CAR we developed for the treatment of ccRCC. The CAR was constructed with an affinity fine-tuned anti-CAIX scFv and the secreted Ab payload is an anti-PDL1 or irrelevant anti-SARS antibody. As can be seen there is markedly improved anti-tumor efficacy of the anti-CAIX CAR secreting anti-PDL1 Ab that acts as an ICI for this cancer.

### Research Plan:

Primary OvCA PDX models from tumor cells that arose from HGSOC ascites have been established 27. Fourteen of these cell lines had growth kinetics suitable for robust in vivo experiments. Two of these will be luciferized and characterized for use in our DFIR CAR-T cell studies. These cell lines will be HLA-typed and we will identify 5/6 or 6/6 HLA matched donors from commercial blood companies for our studies. We will use G-CSF mobilized peripheral blood (PB) and will purify both CD34+ hematopoietic stem cells (HSCs) and T cells using commercial kits for immune-reconstitution and CART generation, respectively. Circa 3-4 months post-reconstruction, when PB hCD45 cells are >25%, mice will be implanted i.p. with the in vivo passaged tumors. At a later timepoint CAR-T cells from the same donor will be injected via tail vein. Mice will be monitored by weekly BLI imaging and will be sacrificed at an appropriate time based on tumor growth. Ascites will be harvested and processed for determination of tumor cell counts. CAR-T cells and TILs will be processed for FACS staining of T cell subset and myeloid lineage and exhaustion markers (PD1, TIM3 and LAG3) and for scRNASeq to characterize changes in the TME.

Each animal study will involve 5 groups; anti-MUC1/anti-MSLN DFIR CAR-T secreting anti-TIGIT/anti-CCR4 BsAb, anti-MUC1/anti-MSLN DFIR CAR-T secreting irrelevant BsAb, irrelevant DFIR CAR-T secreting anti-TIGIT/anti-CCR4 BsAb, irrelevant DFIR CAR-T secreting irrelevant BsAb and untransduced T cells (5 groups, n=6 mice/group). Animals will undergo BLI imaging for luciferase activity weekly beginning with the baseline immediately after tumor implant. Tumor growth will be quantitated by luciferase activity. Two experiments for each of two different tumor cell lines will be performed. (Total mice=144 allowing for 36 mice in each study (accounting for 6 mice per reconstitution to drop out because of death, poor immune reconstruction, infection).

For scRNASeq analysis, bioinformatic pipelines for this project will comprise a combination of tools including alAkazam 28; Shazam 29; Seurat 30; Cumulus 31. We will validate differences in transcriptional states, metabolic state and evolutionary patterns between different CAR-T treatments. Some overlap between subsets will be seen. We will compare these different treatments to validate whether there are phenotypic and functional properties that distinguish anti-MUC1/MSLN DFIR CAR-T cells secreting anti-CCR4/TIGIT BsAbs from other treatments and whether there are differences between the T cell donors.

Results: Without wishing to be bound by theory, anti-MUC1/anti-MSLN DFIR CAR-T secreting anti-TIGIT/anti-CCR4 BsAb will show the greatest anti-tumor activity compared to other groups, followed by anti-MUC1/anti-MSLN DFIR CAR-T secreting irrelevant BsAb. Further, an irrelevant DFIR CAR-T secreting anti-TIGIT/anti-CCR4 BsAb will not have much of a clinical effect since that DFIR CAR-T cells are not targeted and will not undergo expansion. The other two animal groups will show uncontrolled tumor cell growth. Without wishing to be bound by theory, T-cell exhaustion profiles from both FACS staining and scRNASeq will correlate with the anti-tumor activity and support the contribution of the secreted anti-CCR4/TIGIT BsAb on restoring anti-tumor molecular and functional signatures.

Data analysis and statistical considerations:

Tumor growth: Data will be analyzed in GraphPad Prism. BLI will be analyzed using Living Image software.

Flow Cytometry: Flow cytometry data will be analyzed using FlowJo. PBMC and peripheral blood mononuclear (PMN) cells are stained with each individual marker to appropriately compensate for colors.

Single Cell RNA Sequencing: 10× Genomics provides a comprehensive analysis suite for data processing (Cell Ranger) and visualization (Loupe Cell Browser). Furthermore, programs in R like Seurat can further analyze the data output from Cell Ranger.

Tumor growth trajectories will be summarized by averaging tumor growth at each time point across the repeated experiments, for each of the four types of CAR-T therapies and the control therapy (untransduced T cells) within each of the two PDX models. The most promising CAR-T therapy will have the greatest decrease in tumor growth in both PDX models. FACS analysis of T-cell subsets and markers of exhaustion will be performed on weekly bleeds and will be compared between the 5 groups over time. This data will allow us to examine and quantitate the effect of the DFIR CAR-T therapy on T cell differentiation and function over time.

With 6 samples per group we will have 0.8 power to detect differences in the most extreme pair of comparisons (control groups: irrelevant DFIR CAR-T with irrelevant payload or untransduced) between the means equal to 1.8 standard deviations using 2-sample two-tailed t-test at 0.05 level. However, the size of the cohorts will ultimately determine the statistical methods that we use. For example, if we have 4 mice per group for each pairwise comparison, and we will need much larger effect size (difference between groups) for the same power. Likewise, with 4 samples per group we will have 0.8 power to detect differences of the group means equal to 2.3 standard deviations using 2-sample two-tailed t-test at 0.05 level.

The chosen dose of CAR-T cells will set at 1×10e6, a level that has resulted in strong tumor cell killing in other CAR-T/mice cancer models. This dose can be adjusted.

### REFERENCES CITED IN THIS EXAMPLE

1. Webb P M, Jordan S J. Epidemiology of epithelial ovarian cancer. Best Pract Res Clin Obstet Gynaecol. 2017; 41:3-14. Epub 2016 Oct. 17. doi: 10.1016/j.bpobgyn.2016.08.006. PubMed PMID: 27743768. PMCID: NA
2. Agarwal R, Kaye S B. Ovarian cancer: strategies for overcoming resistance to chemotherapy. Nat Rev Cancer. 2003; 3 (7): 502-16. Epub 2003 Jul. 2. doi: 10.1038/nrc1123. PubMed PMID: 12835670. PMCID: NA
3. DiSaia P J, Bloss J D. Treatment of ovarian cancer: new strategies. Gynecol Oncol. 2003; 90 (2 Pt 2): S24-32. Epub 2003 Aug. 21. doi: 10.1016/s0090-8258 (03) 00341-x. PubMed PMID: 12928003.PMCID: PMC747367
4. Disis M L, Rivkin S. Future directions in the management of ovarian cancer. Hematol Oncol Clin North Am. 2003; 17 (4): 1075-85. Epub 2003 Sep. 10. doi: 10.1016/s0889-8588 (03) 00054-6. PubMed PMID: 12959192. PMCID: NA
5. Borella F, Ghisoni E, Giannone G, Cosma S, Benedetto C, Valabrega G, Katsaros D. Immune Checkpoint Inhibitors in Epithelial Ovarian Cancer: An Overview on Efficacy and Future Perspectives. Diagnostics (Basel). 2020; 10 (3). Epub 2020 Mar. 12. doi: 10.3390/diagnostics10030146. PubMed PMID: 32156035; PMCID: PMC7151145.
6. Lee D W, Barrett D M, Mackall C, Orentas R, Grupp S A. The future is now: chimeric antigen receptors as new targeted therapies for childhood cancer. Clin Cancer Res. 2012; 18 (10): 2780-90. Epub 2012 May 17. doi: 10.1158/1078-0432.Ccr-11-1920. PubMed PMID: 22589486; PMCID: PMC4119811.
7. Yip A, Webster R M. The market for chimeric antigen receptor T cell therapies. Nature Reviews Drug Discovery. 2018; 17:161-2. PMCID: NA
8. Srivastava S, Riddell S R. Engineering CAR-T cells: Design concepts. Trends Immunol. 2015; 36 (8): 494-502. Epub 2015 Jul. 15. doi: 10.1016/j.it.2015.06.004. PubMed PMID: 26169254; PMCID: PMC4746114.
9. Maude S L, Frey N, Shaw P A, Aplenc R, Barrett D M, Bunin N J, Chew A, Gonzalez V E, Zheng Z, Lacey S F, Mahnke Y D, Melenhorst J J, Rheingold S R, Shen A, Teachey D T, Levine B L, June C H, Porter D L, Grupp S A. Chimeric antigen receptor T cells for sustained remissions in leukemia. N Engl J Med. 2014; 371 (16): 1507-17. Epub 2014 Oct. 16. doi: 10.1056/NEJMoa1407222. PubMed PMID: 25317870; PMCID: PMC4267531.
10. Zhu Y, Tan Y, Ou R, Zhong Q, Zheng L, Du Y, Zhang Q, Huang J. Anti-CD19 chimeric antigen receptor modified T cells for B-cell malignancies: a systematic review of efficacy and safety in clinical trials. Eur J Haematol. 2016; 96 (4): 389-96. Epub 2015 Jun. 27. doi: 10.1111/ejh. 12602. PubMed PMID: 26115358. PMCID: NA
11. Kakarla S, Gottschalk S. CAR T cells for solid tumors: armed and ready to go? Cancer J. 2014; 20 (2): 151-5. Epub 2014 Mar. 29. doi: 10.1097/ppo.0000000000000032. PubMed PMID: 24667962; PMCID: PMC4050065.
12. Deng J, Wang L, Chen H, Li L, Ma Y, Ni J, Li Y. The role of tumour-associated MUC1 in epithelial ovarian cancer metastasis and progression. Cancer Metastasis Rev. 2013; 32 (3-4): 535-51. Epub 2013 Apr. 24. doi: 10.1007/s10555-013-9423-y. PubMed PMID: 23609751. PMCID: NA
13. Hilliard T S. The Impact of Mesothelin in the Ovarian Cancer Tumor Microenvironment. Cancers (Basel). 2018; 10 (9). Epub 2018 Aug. 24. doi: 10.3390/cancers10090277. PubMed PMID: 30134520; PMCID: PMC6162689.
14. Wood K J, Sakaguchi S. Regulatory T cells in transplantation tolerance. Nat Rev Immunol. 2003; 3 (3): 199-210. Epub 2003 Mar. 27. doi: 10.1038/nri1027. PubMed PMID: 12658268. PMCID: NA
15. Curiel T J, Coukos G, Zou L, Alvarez X, Cheng P, Mottram P, Evdemon-Hogan M, Conejo-Garcia J R, Zhang L, Burow M, Zhu Y, Wei S, Kryczek I, Daniel B, Gordon A, Myers L, Lackner A, Disis M L, Knutson K L, Chen L, Zou W. Specific recruitment of regulatory T cells in ovarian carcinoma fosters immune privilege and predicts reduced survival. Nat Med. 2004; 10 (9): 942-9. Epub 2004 Aug. 24. doi: 10.1038/nm1093. PubMed PMID: 15322536. PMCID: NA
16. Iellem A, Mariani M, Lang R, Recalde H, Panina-Bordignon P, Sinigaglia F, D'Ambrosio D. Unique chemotactic response profile and specific expression of chemokine receptors CCR4 and CCR8 by CD4 (+) CD25 (+) regulatory T cells. J Exp Med. 2001; 194 (6): 847-53. Epub 2001 Sep. 19. doi: 10.1084/jem.194.6.847. PubMed PMID: 11560999; PMCID: PMC2195967.
17. Levings M K, Sangregorio R, Roncarolo M G. Human cd25 (+) cd4 (+) t regulatory cells suppress naive and memory T cell proliferation and can be expanded in vitro without loss of function. J Exp Med. 2001; 193 (11): 1295-302. Epub 2001 Jun. 8. doi: 10.1084/jem. 193.11.1295. PubMed PMID: 11390436; PMCID: PMC2193376.
18. Sakaguchi S, Miyara M, Costantino C M, Hafler D A. FOXP3+ regulatory T cells in the human immune system. Nat Rev Immunol. 2010; 10 (7): 490-500. Epub 2010 Jun. 19. doi: 10.1038/nri2785. PubMed PMID: 20559327. PMCID: NA
19. Kloss C C, Condomines M, Cartellieri M, Bachmann M, Sadelain M. Combinatorial antigen recognition with balanced signaling promotes selective tumor eradication by engineered T cells. Nat Biotechnol. 2013; 31 (1): 71-5. Epub 2012 Dec. 18. doi: 10.1038/nbt.2459. PubMed PMID: 23242161; PMCID: PMC5505184.
20. Hegde M, Mukherjee M, Grada Z, Pignata A, Landi D, Navai S A, Wakefield A, Fousek K, Bielamowicz K, Chow K K, Brawley V S, Byrd T T, Krebs S, Gottschalk S, Wels W S, Baker M L, Dotti G, Mamonkin M, Brenner M K, Orange J S, Ahmed N. Tandem CAR T cells targeting HER2 and IL13Rα2 mitigate tumor antigen escape. J Clin Invest. 2016; 126 (8): 3036-52. Epub 2016 Jul. 19. doi: 10.1172/jci83416. PubMed PMID: 27427982; PMCID: PMC4966331.
21. Rafiq S, Hackett C S, Brentjens R J. Engineering strategies to overcome the current roadblocks in CAR T cell therapy. Nat Rev Clin Oncol. 2020; 17 (3): 147-67. Epub 2019 Dec. 19. doi: 10.1038/s41571-019-0297-y. PubMed PMID: 31848460; PMCID: PMC7223338 consultant for JUNO Therapeutics/Celgene. The other authors declare no competing interests.
22. Han T, Abdel-Motal U M, Chang D K, Sui J, Muvaffak A, Campbell J, Zhu Q, Kupper T S, Marasco W A. Human anti-CCR4 minibody gene transfer for the treatment of cutaneous T-cell lymphoma. PLOS One. 2012; 7 (9): e44455. Epub 2012 Sep. 14. doi: 10.1371/journal.pone.0044455. PubMed PMID: 22973452; PMCID: PMC3433438.
23. Chang D K, Peterson E, Sun J, Goudie C, Drapkin R I, Liu J F, Matulonis U, Zhu Q, Marasco W A. Anti-CCR4 monoclonal antibody enhances antitumor immunity by modulating tumor-infiltrating Tregs in an ovarian cancer xenograft humanized mouse model. Oncoimmunology. 2016; 5 (3): e1090075. Epub 2016 May 4. doi: 10.1080/2162402x.2015.1090075. PubMed PMID: 27141347; PMCID: PMC4839340.
24. Chang D K, Sui J, Geng S, Muvaffak A, Bai M, Fuhlbrigge R C, Lo A, Yammanuru A, Hubbard L, Sheehan J, Campbell J J, Zhu Q, Kupper T S, Marasco W A. Humanization of an anti-CCR4 antibody that kills cutaneous T-cell lymphoma cells and abrogates suppression by T-regulatory cells. Mol Cancer Ther. 2012; 11 (11): 2451-61. Epub 2012 Aug. 8. doi: 10.1158/1535-7163.Mct-12-0278. PubMed PMID: 22869555; PMCID: PMC3496034.
25. Wang Y, Chan L L, Grimaud M, Fayed A, Zhu Q, Marasco W A. High-Throughput Image Cytometry Detection Method for CAR-T Transduction, Cell Proliferation, and Cytotoxicity Assays. Cytometry A. 2020. Epub 2020 Nov. 17. doi: 10.1002/cyto.a.24267. PubMed PMID: 33191639. PMCID: NA
26. Jenkins R W, Aref A R, Lizotte P H, Ivanova E, Stinson S, Zhou C W, Bowden M, Deng J, Liu H, Miao D, He M X, Walker W, Zhang G, Tian T, Cheng C, Wei Z, Palakurthi S, Bittinger M, Vitzthum H, Kim J W, Merlino A, Quinn M, Venkataramani C, Kaplan J A, Portell A, Gokhale P C, Phillips B, Smart A, Rotem A, Jones R E, Keogh L, Anguiano M, Stapleton L, Jia Z, Barzily-Rokni M, Cañadas I, Thai T C, Hammond M R, Vlahos R, Wang E S, Zhang H, Li S, Hanna G J, Huang W, Hoang M P, Piris A, Eliane J P, Stemmer-Rachamimov A O, Cameron L, Su M J, Shah P, Izar B, Thakuria M, LeBoeuf N R, Rabinowits G, Gunda V, Parangi S, Cleary J M, Miller B C, Kitajima S, Thummalapalli R, Miao B, Barbie T U, Sivathanu V, Wong J, Richards W G, Bueno R, Yoon C H, Miret J, Herlyn M, Garraway L A, Van Allen E M, Freeman G J, Kirschmeier P T, Lorch J H, Ott P A, Hodi F S, Flaherty K T, Kamm R D, Boland G M, Wong K K, Dornan D, Paweletz C P, Barbie D A. Ex Vivo Profiling of PD-1 Blockade Using Organotypic Tumor Spheroids. Cancer Discov. 2018; 8 (2): 196-215. Epub 2017 Nov. 5. doi: 10.1158/2159-8290.Cd-17-0833. PubMed PMID: 29101162; PMCID: PMC5809290.
27. Liu J F, Palakurthi S, Zeng Q, Zhou S, Ivanova E, Huang W, Zervantonakis I K, Selfors L M, Shen Y, Pritchard C C, Zheng M, Adleff V, Papp E, Piao H, Novak M, Fotheringham S, Wulf G M, English J, Kirschmeier P T, Velculescu V E, Paweletz C, Mills G B, Livingston D M, Brugge J S, Matulonis U A, Drapkin R. Establishment of Patient-Derived Tumor Xenograft Models of Epithelial Ovarian Cancer for Preclinical Evaluation of Novel Therapeutics. Clin Cancer Res. 2017; 23 (5): 1263-73. Epub 2016 Aug. 31. doi: 10.1158/1078-0432.Ccr-16-1237. PubMed PMID: 27573169; PMCID: PMC5332350.
28. Stern J N, Yaari G, Vander Heiden J A, Church G, Donahue W F, Hintzen R Q, Huttner A J, Laman J D, Nagra R M, Nylander A, Pitt D, Ramanan S, Siddiqui B A, Vigneault F, Kleinstein S H, Hafler D A, O'Connor K C. B cells populating the multiple sclerosis brain mature in the draining cervical lymph nodes. Sci Transl Med. 2014; 6 (248): 248ra107. Epub 2014 Aug. 8. doi: 10.1126/scitranslmed.3008879. PubMed PMID: 25100741; PMCID: PMC4388137.
29. Yaari G, Uduman M, Kleinstein S H. Quantifying selection in high-throughput Immunoglobulin sequencing data sets. Nucleic Acids Res. 2012; 40 (17): e134. Epub 2012 May 30. doi: 10.1093/nar/gks457. PubMed PMID: 22641856; PMCID: PMC3458526.
30. Butler A, Hoffman P, Smibert P, Papalexi E, Satija R. Integrating single-cell transcriptomic data across different conditions, technologies, and species. Nat Biotechnol. 2018; 36 (5): 411-20. Epub 2018 Apr. 3. doi: 10.1038/nbt.4096. PubMed PMID: 29608179; PMCID: PMC6700744. 31. Li B, Gould J, Yang Y, Sarkizova S, Tabaka M, Ashenberg O, Rosen Y, Slyper M, Kowalczyk M S, Villani A C, Tickle T, Hacohen N, Rozenblatt-Rosen O, Regev A. Cumulus provides cloud-based data analysis for largescale single-cell and single-nucleus RNA-seq. Nat Methods. 2020; 17 (8): 793-8. Epub 2020 Jul. 29. doi: 10.1038/s41592-020-0905-x. PubMed PMID: 32719530; PMCID: PMC7437817

### Example 6

### Technical Abstract

Background: Ovarian cancer (OvCA) is the fifth leading cause of cancer deaths in American women. Due to the absence of specific symptoms or signs and the lack of trustworthy screening for early detection, the majority of women with OvCA (60-65%) are diagnosed at a late stage when the cancer has spread beyond the confines of the ovary. Currently, the standard treatment of OvCA is surgical intervention followed by platinum-containing chemotherapy, such as carboplatin plus paclitaxel. Treatment for stage III or IV disease is rarely curative, with 5-year survival rates under 20%. No effective therapy is available for relapsed or metastatic disease that has failed first-line therapy. In addition, several clinical trials with anti-PD (L) 1 checkpoint blockade inhibitors have shown poor results. There is an urgent need for more effective therapies.

Chimeric Antigen Receptor (CAR) T cell therapy is a new type of “living drug”. The FDA approval of 2^{nd} generation CAR-T cell therapies have ushered this new type of cellular immunotherapy into mainstream cancer therapy for hematologic malignancies. However, these results have not been readily translatable to solid tumors. Indeed, several major hurdles have been recognized that limit our ability to achieve “cures” of solid tumors using CAR-T cells. These include on-target, off-tumor toxicity due to sharing of tumor associated antigen (TAA) epitopes on normal tissues, tumor heterogeneity, and the immunosuppressive tumor microenvironment (TME). To address these issues, we have designed Dual-targeted, Fine-tuned, Immune Restoring (DFIR) CAR T cells. We have engineered an anti-MUC1/anti-mesothelin (MSLN) dual-targeted CAR which serves to increase tumor killing efficacy by allowing CAR-T activation with the presence of MUC1 or MSLN to account for solid tumor heterogeneity and to mitigate against single antigen escape. Elevated safety is addressed though fine-tuned CARs such that the affinities of the CAR targeting moieties are tailored to only recognize high density tumor TAAs but not the same antigens expressed as physiologic levels on normal tissues. Immune restoration at the tumor site will occur through secretion of a bispecific antibody (BsAb) at the tumor site that serves as a checkpoint blockade inhibitor. For this project, one arm of the BsAb is directed against TIGIT which is over expressed on tumor antigen-specific CD8+ T cells and CD8+ tumor infiltrating lymphocytes (TILs) from individuals with high-grade serous ovarian cancer and the second arm is against CCR4 which is overexpressed on regulatory T cells (Tregs) that are elevated in the TILs in OvCA.

Without wishing to be bound by theory, antitumor efficacy and safety of CAR-T cell therapy for HGSOC can be greatly improved through dual targeting and fine-tuning of the binding affinity of the anti-MUC1 and anti-MSLN CAR targeting moieties. Further, the DF CAR-T cells can be additionally engineered to secrete a bispecific antibody (BsAb) that targets two of the major immunosuppressive pathways that are operative in HGSOC. The locally secreted BsAb will be directed to TIGIT which is present on exhausted T cells, regulatory T cells (Tregs) and NK cells and CCR4, which is highly expressed on Tregs. Without wishing to be bound by theory, simultaneously blocking these two pathways will reverse immunosuppression and restore local anti-tumor immunity.

### Specific Aims:

Aim 1-in vitro studies on OvCA cell killing efficacy by anti-MUC1/MSLN DFIR CAR T cells that constitutively secrete anti-TIGIT/CCR4 BsAb at the tumor site and to document reversal of T cell exhaustion.

Aim 2-in vivo studies testing DFIR CAR-T cell efficacy in humanized mice (NSG-SGM3) bearing HLA-matched ovarian tumors. Studies will be designed with appropriate control groups to separately assess the effects of anti-MUC1/anti-MSLN DFIR CAR-T cells and their anti-TIGIT/CCR4 BsAb payloads.

Development of dual-targeting of CAR T cells to mitigate against tumor cell heterogeneity and single antigen escape; Development of fine-tuning CARs to elevate safety against killing of normal tissues that express the same antigens, albeit at lower densities; Genetic delivery of a bispecific antibody that can act as a dual checkpoint blockade inhibitor.

Use of PDOTs to characterize DFIR CAR-T cells; Development of humanized mouse model to interrogate the OvCA TME and DFIR CAR-T interventions; Use of scRNAseq to catalogue and quantitate changes in TME that result from DFIC CAR-T therapy.

Without wishing to be bound by theory, these studies are designed to provide a life-long therapy as a living drug that can achieve cures of OvCA. This cellular combination immunotherapy is formulated to be administered as a one-time, single agent which can also decrease the burden on the patient, clinician, and payer.

### Lab Abstract

Ovarian cancer is often diagnosed late, and treatment for stage III or IV disease is rarely curative with 5-year survival rates under 20%. However, no effective therapy is available for relapsed or metastatic disease that has failed first-line therapy. Recent trials involving the new immunotherapy agents known as immune checkpoint inhibitors such as anti-PD (L) 1 monoclonal antibodies have also shown poor clinical results. Active and retired women of the military deserve the very best treatment that we can offer in return for their valiant service to our country. There is an urgent need for more effective therapies to treat our military women and our proposal offers hope that a breakthrough therapy for high-grade serous ovarian cancer (HGSOC) can be discovered.

Chimeric Antigen Receptor (CAR) T cell therapy is a new type of “living drug” that holds promise for the “Cure” of OvCA. This therapy takes advantage of our own immune cells and engineers them to recognize and kill cancer cells. These CAR-T therapies have shown clinically meaningful results for the treatment of hematologic malignancies which has resulted in 3 FDA approved drugs, however, these findings have not been readily translated to solid tumors. Several major hurdles have been recognized that limit our ability to achieve “cures” of solid tumors using CAR-T cells. To address these issues, we have designed Dual-targeted, Fine-tuned, Immune Restoring (DFIR) CAR-T cells for the treatment of solid tumors with an emphasis on improving anti-tumor efficacy and patient safety.

As described herein, we will construct DFIR CAR-T cells and validate their ability to kill HGSOC tumors in tissue culture. This therapy also restores anti-tumor immunity of the patient immune cells that arrive at the tumor but quickly become exhausted. Also, DFIR CAR-T treatment studies in humanized mice will validate that this treatment “cures” the HGSOC tumors. Humanized mice are mice that are engineered to carry a human immune system. Our analyses will also be aimed at understanding the mechanism by which the DFIR CAR-T cells work to restore anti-tumor immunity and is an important part of our studies.

Finally, DFIR CAR-T cells can remain in the patient for life where they can constantly monitor by a process we call “immune surveillance” to prevent new HGSOC cells from arising. This cellular combination immunotherapy is formulated to be administered as a one-time, single agent, which can also decrease the burden on the patient, clinician, and payer. Without wishing to be bound by theory, this DFIR CAR-T therapy offers a treatment of our warfighters and military who deserve the very best.

### Impact Statement

Ovarian cancer is often diagnosed late, and treatment for stage III or IV disease is rarely curative, with 5-year survival rates under 20%. The disease strikes women in the US, including active and retired women of the military who deserve the very best treatment that we can offer in return for their valiant service to our country. However no effective therapy is available for relapsed or metastatic disease that has failed first-line therapy. There is an urgent need for more effective therapies and our proposal offers hope that a breakthrough therapy for HGSOC can be discovered.

Chimeric Antigen Receptor (CAR) T cell therapy is a new type of “living drug” that holds promise for the “Cure” of OvCA. This therapy takes advantage of our own immune cells and engineers them to recognize and kill cancer cells. These CAR-T therapies have shown clinically meaningful results for the treatment of hematologic malignancies which has resulted in 3 FDA approved drugs, however, these findings have not been readily translated to solid tumors. Several major hurdles have been recognized that limit our ability to achieve “cures” of solid tumors using CAR-T cells. These include on-target, off-tumor toxicity due to sharing of tumor associated antigen (TAA) epitopes on normal tissues, tumor heterogeneity, and the immunosuppressive tumor microenvironment (TME). To address these issues, we have constructed Dual-targeted, Fine-tuned, Immune Restoring (DFIR) CAR-T cells for the treatment of solid tumors with an emphasis on improving anti-tumor efficacy and safety.

MUC1 and MSLN are overexpressed on OvCA, and HGSOC. There is heterogeneity of their expression so that not all tumor cells in a patient will express both TAAs. The TME has “public” signatures that are common to women with OvCA that can be targetable with immune checkpoint blockade inhibitors (ICIs). Our DFIR CAR-T cells will be able to kill a heterogeneous tumor cell population because of our “and/or” gating strategy that is allowed for by dual anti-MUC1 and anti-MSLN CAR-T targeting. Specifically, dual targeting will promote killing of both singly (MUC1 or MSLN) positive tumor cells and doubly (MUC1 and MSLN1) positive tumor cells. Our DFIR CAR-T cells are also anti-TIGIT/CCR4 BsAb secreting cells. Both TIGIT and CCR4 are targetable checkpoint molecules and are found to be highly expressed on TILs and tumor associated lymphocytes (TALs). In addition, blocking TIGIT/CD155 interactions will reverse cytotoxic T cell exhaustion, block Treg immunosuppressive function and restore NK killing function. Blocking CCR4 interaction will also prevent the accumulation of Tregs at the OvCA site through their blockade of binding of the CCL17/CCL22 chemokines that are secreted by the tumor cells. Our project involves both in vitro and in vivo studies in humanized mice and utilizes the most up-to-date technologies to evaluate our new therapy.

Finally, this cellular combination immunotherapy is formulated to be administered as a one-time, single agent, which can also decrease the burden on the patient, clinician, and payer. In addition, the anti-TIGIT/CCR4 BsAb, that acts as a ICIs is delivered directly to the tumor site to maximize the effects in the TME while minimizing any systemic toxicity. The CAR-T cells do not disappear after they kill the tumor cells, rather, they stay on to live in the patient to stop any chance of a disease recurrence.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

What is claimed:

1. An antibody or antigen-binding fragment thereof that binds to a mesothelin protein or fragment thereof, comprising a heavy chain, light chain, or both a heavy chain and a light chain,
wherein the heavy chain comprises a
HCDR1 comprising
(SEQ ID NO: 31)
GYTLTTNG,
(SEQ ID NO: 35)
GGTFSSDT,
(SEQ ID NO: 38)
GFTFDDYA,
(SEQ ID NO: 46)
GFKFTDYY,
or
(SEQ ID NO: 49)
GYTFTHYA,
HCDR2 comprising
(SEQ ID NO: 32)
VSPYNGHI,
(SEQ ID NO: 34)
VNPYNGHI,
(SEQ ID NO: 36)
INPNSGGT,
(SEQ ID NO: 39)
ISWNSGSI,
(SEQ ID NO: 41)
ISWNNGSI,
(SEQ ID NO: 43)
ISWNSGNI,
(SEQ ID NO: 47)
INTSSNHI,
or
(SEQ ID NO: 50)
IHAGNGNS,
HCDR3 comprising
(SEQ ID NO: 33)
ARVNRANYYGMDV,
(SEQ ID NO: 37)
ARESALGGSYPLSF,
(SEQ ID NO: 40)
AKDPSSSWLAGAFDI,
(SEQ ID NO: 42)
AKDPSTSWLAGAFDI,
(SEQ ID NO: 44)
AKDAGSSGYFNAFDI,
(SEQ ID NO: 45)
AKSPSSNWYPDAFDI,
(SEQ ID NO: 48)
ARGASWGPL,
or
(SEQ ID NO: 51)
AREVGHGMDV,
or a combination of CDRs thereof;
wherein the light chain comprises a
LCDR1 comprising
(SEQ ID NO: 52)
QSLLRSNGYNY,
(SEQ ID NO: 59)
QSVSSSY,
(SEQ ID NO: 62)
SLRRFY,
(SEQ ID NO: 65)
SLIRFY,
(SEQ ID NO: 67)
SKRAYY,
(SEQ ID NO: 70)
SLRNYY,
(SEQ ID NO: 73)
NIGSKS,
or
(SEQ ID NO: 76)
SSNIGAGYD,
LCDR2 comprising
(SEQ ID NO: 53)
LGS,
(SEQ ID NO: 60)
GAS,
(SEQ ID NO: 63)
GKN,
(SEQ ID NO: 71)
AKN,
(SEQ ID NO: 74)
DDS,
or
(SEQ ID NO: 77)
GNT,
LCDR3 comprising
(SEQ ID NO: 54)
MQSSTNSAH,
(SEQ ID NO: 55)
MQALQTPLT,
(SEQ ID NO: 56)
MQALQTPL,
(SEQ ID NO: 57)
MQALQTPLT,
(SEQ ID NO: 58)
HASSTNSAH,
(SEQ ID NO: 61)
LQDDSYPLT,
(SEQ ID NO: 64)
NSRDSDGNHVF,
(SEQ ID NO: 66)
NSQDRDGNHVF,
(SEQ ID NO: 68)
NSRDSSGNHLGV,
(SEQ ID NO: 69)
NSRDSSGNHLGS,
(SEQ ID NO: 72)
SSRDSSDNHVV,
(SEQ ID NO: 75)
QSADYNWLCD,
or
(SEQ ID NO: 78)
QSYDSSLSGYV,
or a combination of CDRs thereof.

2. The antibody of claim 1, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

3. An antibody as described herein.

4. A therapeutic antibody that binds mesothelin comprising a variable domain and a constant domain, wherein the constant domain is IgG and the variable domain comprises a framework region and a complementary determining means for binding mesothelin.

5. The antibody of claim 4, wherein the constant region is IgG1 or IgG4.

6. An isolated antibody or fragment thereof that binds to a human mesothelin protein comprising:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

7. An isolated scFv antibody that binds to a human mesothelin protein comprising:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

8. An isolated antibody or fragment thereof that binds to a human mesothelin protein, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

9. An isolated scFv that binds to a human mesothelin protein, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

10. A F (ab) that binds mesothelin, comprising:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

11. A VhH that binds mesothelin, comprising:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51.

12. An isolated bispecific antibody, comprising the fragment of any one of claims 1-11 and a second antigen-binding fragment having specificity to a molecule on an immune cell.

13. The bispecific antibody of claim 12, wherein the molecule is selected from the group consisting of CCR4, B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, and KIR.

14. The bispecific antibody of claim 12, wherein the fragment and the second fragment each is independently selected from a F_{ab} fragment, a single-chain variable fragment (scFv), or a single-domain antibody.

15. The bispecific antibody of claim 12, further comprising a Fc fragment.

16. A bispecific T cell engager (BiTE) that binds to a human mesothelin protein comprising:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

17. A bispecific T cell engager (BiTE) that binds to a human mesothelin protein, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

18. The antibody of any one of claims 1-11 having at least 90% sequence identity.

19. A pharmaceutical composition, comprising: the antibody of any one of claims 1-11, and one or more pharmaceutically acceptable carriers, diluents, or excipients.

20. The pharmaceutical composition of claim 19, comprising:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

21. The pharmaceutical composition of claim 19, comprising a heavy chain variable region (HCVR) and a light chain variable region (LCVR), wherein the amino acid sequence of the HCVR is SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, or SEQ ID NO: 29, and the amino acid sequence of the LCVR is SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, or SEQ ID NO: 30.

22. A nucleic acid encoding the antibody or fragment according to any one of claims 1-11.

23. A nucleic acid encoding the bispecific antibody according to any one of claims 12-15.

24. A nucleic acid encoding the BiTE according to any one of claims 16-17.

25. An engineered cell comprising a chimeric antigen receptor, wherein the chimeric antigen receptor comprises an extracellular ligand binding domain that is specific for an antigen on the surface of a cancer cell, wherein the antigen comprises mesothelin, wherein the extracellular ligand binding domain comprises an antibody or fragment thereof, where the antibody or fragment thereof comprises:
(a) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(b) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 54; or
(c) a VH CDR1 comprising the amino acid sequence of SEQ ID NO: 31, a VH CDR2 comprising the amino acid sequence of SEQ ID NO: 34, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 55; or
(d) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 56; or
(e) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 57; or
(f) a VH CDR1 comprising the amino acid sequence of SEQ ID NO: 31, a VH CDR2 comprising the amino acid sequence of SEQ ID NO: 32, a VH CDR3 comprising the amino acid sequence of SEQ ID NO: 33, a VL CDR1 comprising the amino acid sequence of SEQ ID NO: 52, a VL CDR2 comprising the amino acid sequence of SEQ ID NO: 53, and a VL CDR3 comprising the amino acid sequence of SEQ ID NO: 58; or
(g) a VH CDR1 comprising the amino acid sequence of SEQ ID NO: 35, a VH CDR2 comprising the amino acid sequence of SEQ ID NO: 36, a VH CDR3 comprising the amino acid sequence of SEQ ID NO: 37, a VL CDR1 comprising the amino acid sequence of SEQ ID NO: 59, a VL CDR2 comprising the amino acid sequence of SEQ ID NO: 60, and a VL CDR3 comprising the amino acid sequence of SEQ ID NO: 61; or
(h) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64; or
(i) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 40, a VL CDR1 comprising the amino acid sequence of SEQ ID NO: 62, a VL CDR2 comprising the amino acid sequence of SEQ ID NO: 63, and a VL CDR3 comprising the amino acid sequence of SEQ ID NO: 64;
(j) a VH CDR1 comprising the amino acid sequence of SEQ ID NO: 38, a VH CDR2 comprising the amino acid sequence of SEQ ID NO: 41, a VH CDR3 comprising the amino acid sequence of SEQ ID NO: 42, a VL CDR1 comprising the amino acid sequence of SEQ ID NO: 65, a VL CDR2 comprising the amino acid sequence of SEQ ID NO: 63, and a VL CDR3 comprising the amino acid sequence of SEQ ID NO: 66;
(k) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 68;
(l) a VH CDR1 comprising the amino acid sequence of SEQ ID NO: 38, a VH CDR2 comprising the amino acid sequence of SEQ ID NO: 43, a VH CDR3 comprising the amino acid sequence of SEQ ID NO: 44, a VL CDR1 comprising the amino acid sequence of SEQ ID NO: 67, a VL CDR2 comprising the amino acid sequence of SEQ ID NO: 63, and a VL CDR3 comprising the amino acid sequence of SEQ ID NO: 69;
(m) a VH CDR1 comprising the amino acid sequence of SEQ ID NO: 38, a VH CDR2 comprising the amino acid sequence of SEQ ID NO: 39, a VH CDR3 comprising the amino acid sequence of SEQ ID NO: 45, a VL CDR1 comprising the amino acid sequence of SEQ ID NO: 70, a VL CDR2 comprising the amino acid sequence of SEQ ID NO: 71, and a VL CDR3 comprising the amino acid sequence of SEQ ID NO: 72;
(n) a VH CDR1 comprising the amino acid sequence of SEQ ID NO: 46, a VH CDR2 comprising the amino acid sequence of SEQ ID NO: 47, a VH CDR3 comprising the amino acid sequence of SEQ ID NO: 48, a VL CDR1 comprising the amino acid sequence of SEQ ID NO: 73, a VL CDR2 comprising the amino acid sequence of SEQ ID NO: 74, and a VL CDR3 comprising the amino acid sequence of SEQ ID NO: 75; or
(o) a VH CDR1 comprising the amino acid sequence of SEQ ID NO: 49, a VH CDR2 comprising the amino acid sequence of SEQ ID NO: 50, a VH CDR3 comprising the amino acid sequence of SEQ ID NO: 51, a VL CDR1 comprising the amino acid sequence of SEQ ID NO: 76, a VL CDR2 comprising the amino acid sequence of SEQ ID NO: 77, and a VL CDR3 comprising the amino acid sequence of SEQ ID NO: 78.

26. The engineered cell of claim 25, wherein the antibody or fragment thereof comprises a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

27. The engineered cell of claim 25, wherein the cell comprises a T cell, an NK cell, an NKT cell, an iPS cell, an iPS-derived cell, a cell line, or a B cell.

28. The engineered cell of claim 25, wherein the cell comprises a CD4+, CD8+, CD3+ pan T cells, or any combination thereof.

29. A method of treating cancer comprising administering to a subject in need thereof an effective amount of (a) the antibody of any one of claims 1-11, or (b) the pharmaceutical composition of claim 19.

30. A method of inhibiting mesothelin in a subject, comprising administering to the subject an effective amount of (a) the antibody of any one of claims 1-11, or (b) the pharmaceutical composition of claim 19.

31. The method of claim 30, comprising a heavy chain variable region comprising an amino acid sequence selected from the group consisting of SEQ ID NOS: 1, 3, 5, 7, 9, 11, 13, 15, 17, 19, 21, 23, 25, 27 and 29, or a sequence at least 90% identical thereto, and a light chain variable region comprising an amino acid sequence selected from the group consisting of SEQ ID NOS: 2, 4, 6, 8, 10, 12, 14, 16, 18, 20, 22, 24, 26, 28 and 30, or a sequence at least 90% identical thereto.

32. Use of (a) the antibody of any one of claims 1-11, or (b) the pharmaceutical composition of claim 19 for treating cancer.

33. The antibody of any one of claims 1-11, or (b) the pharmaceutical composition of claim 19 for use in therapy.

34. The antibody of any one of claims 1-11, or (b) the pharmaceutical composition of claim 19 for use in treating cancer.
